(19) United States
(12) Patent Application Publication
(10) Pub. No.: US 2022/0401751 A1
(43) Pub. Date: Dec. 22, 2022

(54) Thalamic Input to Orbitofrontal Cortex Drives Brain-Wide, Frequency-Dependent Inhibition Mediated By GABA and Zona Incerta

(71) Applicant: The Board of Trustees of the Leland Stanford Junior University, Stanford, CA (US)

(72) Inventors: Jin Hyung Lee, Palo Alto, CA (US); Andrew J. Weitz, Bishop, CA (US); Hyun Joo Lee, Palo Alto, CA (US)

(21) Appl. No.: 17/642,632

(22) PCT Filed: Sep. 24, 2020
(86) PCT No.: PCT/US2020/052542

§ 371 Date: Mar. 11, 2022

Related U.S. Application Data
(60) Provisional application No. 62/905,557, filed on Sep. 25, 2019.

(51) Int. Cl. A61N 5/06; G01R 33/48

(52) U.S. Cl. CPC A61N 5/0622; G01R 33/4806; A61N 2005/0626; A61N 2005/0663; A61N 2005/0652; A61N 5/067

(57) ABSTRACT

Provided herein are methods and systems for modulating temporal patterns of neuronal activity in the brain. A method of the present disclosure may include using optogenetics to stimulate a one or more of thalamocortical projections, thalamic relay neurons, cortical projection neurons, cell bodies in a thalamic submedial nucleus, and cell bodies in the VLO in the brain, in conjunction with fMRI of different regions of the brain to directly visualize the global influence of the VLO's afferent and efferent connections, and characterize how different temporal patterns of activity in the VLO circuit affect brain dynamics by driving its input and output at distinct frequencies.

### CROSS REFERENCE TO RELATED APPLICATION

This application claims priority pursuant to 35 U.S.C. § 119(e) to the filing date of U.S. Provisional Patent Application Ser. No. 62/905,557, filed Sep. 25, 2019; the disclosure of which application is incorporated herein by reference.

### STATEMENT REGARDING FEDERALLY SPONSORED RESEARCH

This invention was made with Government support under contracts AG047666, MH114227, NS087159, and NS091461 awarded by the National Institutes of Health. The Government has certain rights in the invention.

### INTRODUCTION

The orbitofrontal cortex (OFC) has been implicated in diverse cognitive and emotional functions. The ventrolateral orbital cortex (VLO), one of five sectors within OFC, stands out for supporting many of these functions. Thalamic input to VLO plays a key role in modulating perceived pain levels during noxious stimuli and supports goal-directed behavior by signaling predictive cues and expected outcome. The VLO is linked to spatial navigation and attention, depression, memory formation, and risk assessment. Cortical afferents also allow VLO to integrate information related to diverse processes. These connections, along with extensive efferent projections, suggest that VLO may act as a global hub, modulating activity across brain circuits. Despite evidence that VLO has a global role in brain function, the circuit mechanisms by which it accomplishes such influence have not been studied directly.

To better understand how VLO supports different behavioral processes, a technical approach is needed capable of controlling individual circuit elements while visualizing the brain-wide response.

### SUMMARY

Provided herein are methods and systems for modulating temporal patterns of neuronal activity in the brain. A method of the present disclosure may include using optogenetics to stimulate a one or more of thalamocortical projections, thalamic relay neurons, cortical projection neurons, cell bodies in a thalamic submedial nucleus, and cell bodies in the ventrolateral orbitofrontal cortex (VLO) in the brain, in conjunction with functional magnetic resonance imaging (fMRI) of different regions of the brain to directly visualize the global influence of the VLO's afferent and efferent connections, and characterize how different temporal patterns of activity in the VLO circuit affect brain dynamics by driving its input and output at distinct frequencies.

### BRIEF DESCRIPTION OF THE DRAWINGS

FIGS. 1A-IG show that optogenetic fMRI reveals robust but divergent responses to thalamocortical stimulation in VLO at 10 and 40 Hz. FIG. 1A. Experimental design for virus injection and thalamocortical stimulation. FIG. 1B. Schematic of 23 coronal slices acquired in Optogenetic fMRI (ofMRI) experiments. FIG. 1C. Design matrix for the block-design stimulation paradigm. (FIGS. 1D-1E). Group-level activation maps during thalamocortical stimulation at 10 (FIG. 1D) and 40 (FIG. 1E) H z (N=11 animals; p<0.05, FWE-corrected). In these and all other activation maps, white triangles indicate site of stimulation; warm colors indicate positive t-scores; cool colors indicate negative t-scores; image numbers correspond to slices shown in panel FIG. 1B. FIGS. 1F-1G. Single-cycle fMRI time series from segmented areas of ipsilateral (FIG. 1F) and contralateral (FIG. 1G) cortex. Horizontal blue lines indicate the stimulation period. Error bars represent mean±s.e.m. across animals (N=11). See also FIGS. 8-12.

FIGS. 2A-2C show that frequency sweep experiments reveal transitions in evoked activity patterns between low and high stimulation frequencies. FIG. 2A. Group-level activation maps during thalamocortical stimulation in VLO at frequencies ranging from 5 to 40 Hz (N=7 animals; p<0.005, uncorrected). FIG. 2B. Quantification of significantly modulated brain volume in the ipsilateral hemisphere. Values represent the fraction of voxels within each ROI that are significantly modulated in group-level activation maps. FIG. 2C. Average single-cycle time series illustrate the frequency-dependent transition from negative to positive responses in sensory, motor, and cingulate cortex. Horizontal blue lines indicate the stimulation period.

FIGS. 3A-3D show widespread negative fMRI signals are not evoked during stimulation of cell bodies in VLO or thalamus. FIGS. 3A-3B. Group-level activation maps during stimulation of cell bodies in VLO at 10 (FIG. 3A) and 40 (FIG. 3B) Hz (N=5 animals; p<0.05, FWE-corrected). FIGS. 3C-3D. Group-level activation maps of evoked responses during stimulation of cell bodies in the submedial nucleus of thalamus at 10 (FIG. 3C) and 40 (FIG. 3D) Hz (N=5 animals; p<0.05, FWE-corrected).

FIGS. 4A-40 show that electrophysiology corroborates frequency-dependent fMRI signals. FIG. 4A. Schematic of single-unit recordings at the site of stimulation in VLO. FIG. 4B. 10 and 40 Hz stimulations drive robust positive fMRI signals at the site of stimulation. FIG. 4C. Peri-event time histograms from a representative unit in VLO that is excited during 10 and 40 Hz stimulation (p=1.2×10⁻⁷ and 7.6×10⁻¹⁰, respectively). Error bars represent mean±s.e.m. over trials. FIG. 4D. Quantification of significant changes in firing rate across recorded units. INC: increase, DEC: decrease, N/C: no change. FIG. 4E. Histograms of stimulus-evoked changes in VLO firing rate (n.s. not significant; p=0.38). FIG. 4F. Schematic of single-unit recordings in the contralateral VLO (cVLO). FIG. 4G. 10 Hz stimulation drives a robust negative fMRI signal in cVLO that largely disappears during 40 Hz stimulation. FIG. 4H. Peri-event time histograms from a representative unit in cVLO. The firing rate decreases during 10 Hz stimulation (p=4.6×10⁻¹³) but does not change during 40 Hz stimulation (p=0.42). FIG. 4I. Quantification of significant changes in firing rate across recorded units in cVLO. FIG. 4J. Histograms of stimulus-evoked changes in cVLO firing rate (p=4.5×10⁻¹). (K) Schematic of single-unit recording in the ipsilateral motor cortex (iMtr). FIG. 4L. 10 Hz thalamocortical stimulation drives a negative fMRI response in iMtr, while 40 Hz stimulation drives a positive fMRI response. FIG. 4M. Peri-event time histograms from a representative unit in iMtr that is inhibited during 10 Hz stimulation (p=3.4×10⁻⁴) but excited during 40 Hz stimulation (p=2.6×10⁻⁶). FIG. 4N. Quantification of significant changes in firing rate across recorded units in iMtr. FIG. 4O. Histograms of stimulus-evoked changes in iMtr firing rate (p=3.9×10⁻²⁹). See also FIGS. 12A-12D.

FIGS. 5A-5G show that remote cortical inhibition driven by low-frequency thalamocortical stimulation is mediated by GABA. FIG. 5A. Schematic of single-unit recording and infusion in cVLO during 10 Hz thalamocortical stimulation. FIG. 5B. Micrograph of the cannula-electrode used to deliver saline and BMI. FIG. 5C. Quantification of significant changes in firing rate during stimulation before and after bolus infusions of saline or BMI. FIG. 5D. Histograms of stimulus-evoked changes in firing rate before and after a single bolus of saline or BMI (p=0.07 and 1.9×10⁻¹⁶, respectively). FIG. 5E. Quantification of baseline firing rate changes after BMI infusion. Error bars represent mean±s.e.m. over trials for each unit and are color-coded according to whether the unit's baseline firing rate significantly increases or decreases. Thick black line indicates mean±s.e.m. across units. FIG. 5F. Timeline of stimulus-evoked changes, averaged over all recorded units, during the twenty trials before and after each bolus infusion. Shaded areas represent one standard deviation. Values reflect the percent signal change in firing rate during each trial's 20 s period of stimulation, relative to the preceding 20 s pre-stimulation period. FIG. 5G. Peri-event time histograms from a representative unit in cVLO. The firing rate decreases during 10 Hz stimulation before and after saline infusion (p=2.0×10⁻⁶ and 1.3×10⁻⁴, respectively) and before BMI infusion (p=1.9×10⁻⁷). After BMI infusion, 10 Hz stimulation no longer causes a significant change in firing rate (p=0.63). Error bars represent mean±s.e.m. over trials.

FIGS. 6A-6E show that pharmacological inactivation of zona incerta reduces remote cortical inhibition driven by low-frequency thalamocortical stimulation. FIG. 6A. Schematic of lidocaine infusion in zona incerta during 10 Hz thalamocortical stimulation and single-unit recordings in cVLO. FIG. 6B. Quantification of significant changes in firing rate evoked by stimulation at baseline and after infusion of saline or lidocaine. FIG. 6C. Timeline of stimulus-evoked changes in firing rate, averaged over units that do not exhibit a significant decrease in firing rate following lidocaine infusion. Shaded areas represent one standard deviation. Values reflect the percent signal change in firing rate during each trial's 20 s period of stimulation, relative to the preceding 20 s pre-stimulation period. FIG. 6D. Left, Histograms of stimulus-evoked changes in cVLO firing rate at baseline, post-saline infusion, and post-lidocaine infusion. Right, Corresponding group means with 95% confidence intervals and post-hoc ANOVA comparisons (*** p<0.001). FIG. 6E. Peri-event time histograms from a representative unit in contralateral VLO. The firing rate decreases during 10 Hz stimulation before and after incertal saline infusion (p=3.9×10⁻⁵ and 1.4×10⁻³, respectively). After lidocaine infusion, the cell no longer exhibits a significant change in firing rate (p=0.31). Error bars represent mean±s.e.m. over trials. See also FIG. 14A-14I.

FIGS. 7A-7H show that optical silencing of zona incerta eliminates the remote cortical inhibition driven by low-frequency thalamocortical stimulation. FIG. 7A. Schematic of single-unit recordings in cVLO and zona incerta (ZI) during 10 Hz thalamocortical stimulation and concurrent silencing of ZI with eNpHR. FIG. 7B. Stimulation paradigm used to assess zona incerta's role in mediating widespread inhibition. FIG. 7C. Quantification of significant changes in ZI firing rate evoked by 10 Hz thalamocortical stimulation with and without eNpHR activation. FIG. 7D. Quantification of significant changes in cVLO firing rate evoked by 10 Hz thalarnocortical stimulation with and without incertal silencing. FIGS. 7E-7F. Histograms of stimulus-evoked changes in ZI (FIG. 7E) and cVLO (FIG. 7F) firing rate (p=2.2×10⁻⁹ and 3.8×10⁻⁷, respectively). FIG. 7G-7H. Peri-event time histograms from representative units in ZI (FIG. 7G) and cVLO (FIG. 7H). The ZI unit's firing rate increases during 10 Hz thalamocortical stimulation (p=6.1×10⁻⁵) but decreases when this is paired with eNpHR activation (p=8.6×10⁻⁴). The cVLO unit's firing rate decreases during 10 Hz thalamocortical stimulation (p=0.030) but does not change when this is paired with eNpHR activation (p=0.23). See also FIGS. 14A-14I.

FIGS. 8A-8D show that stimulation was genetically and spatially targeted to thalamocortical projections in the ventrolateral subdivision of VLO; Related to FIGS. 1A-1G. FIG. 8A. Confocal imaging at the site of injection confirms ChR2-EYFP is expressed in cell bodies of thalamic neurons (white arrowheads). 29% of cells identified within the bulk injection area were ChR2-EYFP-positive (N=2 animals, 343 cells). FIGS. 8B-8C. Confocal (FIG. 8B) and fluorescence (FIG. 8C) imaging in VLO confirms the presence of ChR2-EYFP-positive neuronal processes. ChR2-EYFP-positive cell bodies are not observed, verifying that stimulation was restricted to thalamocortical projections. OLF: olfactory bulb. Note that a secondary antibody, which emits in the red channel, was used to amplify the endogenous EYFP signal. This signal was mapped to the green channel for consistency with standard visualizations of EYFP. (FIG. 8D) Representative T2-weighted MRI scans used to confirm stimulation location in cortex. Arrows mark the location of light delivery at the fiber optic implant tip (left, coronal; right, sagittal).

FIGS. 9A-9D show that fMRI activations driven by thalamocortical stimulation were highly consistent across scans and subjects; Related to FIGS. 1A-1G. FIG. 9A. Single-scan activation maps in response to 40 Hz thalamocortical stimulation for a representative animal (p<0.001, uncorrected). Each scan represents a −7 minute acquisition collected within the same session. White triangles indicate the site of stimulation. Image numbers correspond to coronal slices shown in FIG. 1B. FIG. 9B. Average fMRI time series measured at the site of stimulation (LPFC) and ipsilateral thalamus illustrate the high degree of consistency in evoked responses over repeated trials. Time series come from the same scans shown in (FIG. 9A). FIG. 9C. Activation maps in response to 40 Hz stimulation for each of the 11 animals reported in FIGS. 1A-1G (p<0.001, uncorrected). FIG. 9D. Average fMRI time series measured at the ipsilateral LPFC and thalamus for each animal, illustrating the high degree of inter-subject reproducibility. Time series come from the same scans as shown in FIG. 9C.

FIGS. 10A-10E show quantitative, ROI-based characterization of fMRI responses evoked during thalamocortical stimulation; Related to FIGS. 1A-1G. FIG. 10A. Brain-wide fMRI activations were segmented according to anatomical regions of interest (ROIs) for quantitative analysis of spatiotemporal properties. Segmented ROIs are overlaid as colored regions on the average structural MRI image. FIGS. 10B, 10D. Quantification of modulated voxels in ipsilateral (FIG. 10B) and contralateral (FIG. 10D) regions of interest during 10 and 40 Hz stimulation. Ipsilateral volume is significantly greater during 40 Hz stimulation, while contralateral volume is significantly greater during 10 Hz stimulation (*p<0.05, **p<0.005, ***p<0.001). Red lines indicate values from individual animals. Black lines represent the mean. FIGS. 10C and 10E. Quantification of ΣfMRI values in ipsilateral (FIG. 10C) and contralateral (FIG. 10E.) regions of interest with significant differences from zero marked with asterisks. Three ipsilateral regions—sensory, motor, and cingulate cortex—switch from a significant negative response at 10 Hz to a significant positive response at 40 Hz (t). Contralateral ΣfMRI values are significantly negative during 10 Hz stimulation, but not significantly different from zero during 40 Hz stimulation. Values with error bars represent mean±s.e.m.

FIGS. 11A-11D show that the frequency-dependent effects of thalamocortical projection stimulation are preserved when pulse width (PW) is held constant; Related to FIGS. 1A-1G. (A) Activation maps from a representative animal during 10 and 40 Hz thalamocortical stimulation in VLO using a constant pulse width of 3 ms (p<0.001, uncorrected). White triangles on slice 6 indicate the approximate site of stimulation. Warm colors indicate positive t-scores, while cool colors indicate negative t-scores. Image numbers correspond to coronal slices shown in FIG. 1B. (B) Quantification of total fMRI modulation volume in ipsilateral and contralateral cortex (N=4 animals). Thin gray lines correspond to individual animals. Black lines represent the mean. Values were summed over cortical ROIs. (C) Quantification of ΣfMRI values for ipsilateral ROIs. Error bars represent mean±s.e.m. over animals. (D) Time series from the ipsilateral and contralateral somatosensory cortex. Thin lines indicate the response of individual animals. Thicker lines represent the mean.

FIGS. 12A-12D show that animal-specific electrophysiology results reflect the frequency-dependent trends reported in the main text; Related to FIGS. 4A-40. Each column represents a different animal used for single-unit recordings at the site of stimulation in VLO (FIG. 12A), in the contralateral VLO (FIG. 12B), or in the ipsilateral motor cortex (FIG. 12C).

FIGS. 13A-13H show that stimulus-evoked activity in the thalamic reticular nucleus (TRN) is greater during 40 Hz thalamocortical stimulation than during 10 Hz stimulation; Related to FIGS. 5A-5G. FIGS. 13A, 13E. Schematic of single-unit recording locations in ipsilateral (FIG. 13A) and contralateral (FIG. 13E) TRN during thalamocortical stimulation in VLO. (FIG. 13B) Quantification of significant changes in firing rate in the ipsilateral TRN. More units exhibit a significant increase in firing rate during 40 Hz stimulation. INC: increase, DEC: decrease, N/C: no change. (FIG. 13C) Histograms of stimulus-evoked changes in firing rate within the ipsilateral TRN during 10 and 40 Hz stimulation (p=5.3×10−12). (FIG. 13D) Perievent time histograms from a representative unit in ipsilateral TRN that exhibits a significant increase in firing rate during 40 Hz stimulation (p=1.6×10−4), but no change during 10 Hz stimulation (p=0.39; n.s. not significant). Error bars represent mean±s.e.m. over trials. (FIG. 13F) Quantification of significant changes in firing rate in the contralateral TRN. Activity preferentially decreases during 10 Hz stimulation. (FIG. 13G) Histograms of stimulus-evoked changes in firing rate within the contralateral TRN during 10 and 40 Hz stimulation (p=6.6×10−31). (FIG. 13H) Peri-event time histograms from a representative unit in contralateral TRN that exhibits a significant decrease in firing rate during 10 Hz stimulation (p=7.7×10−8) but a significant increase in firing rate during 40 Hz stimulation (p=0.020).

FIGS. 14A-14I show the methodological details of zona interta targeting and control; Related to FIGS. 6A-6E and FIGS. 7A-7H. (FIGS. 14A-14C) Stereotactic targeting is accurately localized to zona incerta (ZI). (FIG. 14A) To assess the accuracy of stereotactic targeting in zona incerta, bilateral implants were inserted to the target coordinate [−3.96 mm AP, ±2.75 mm ML, −7.20 mm DV] in a separate cohort of animals (N=9). The resulting implant locations were identified with MRI. Individual implants, represented in the schematics as red circles, were all located directly above or within the zona incerta [average location: −3.92 mm AP, ±2.79 mm ML, −7.21 mm DV]. (FIG. 14B) High-resolution ex vivo MRI scans confirmed the correct placement of the infusion cannula in zona incerta during lidocaine hydrochloride experiments. Region outlines for zona incerta and the below white matter tracts are overlaid for clarity. Fast low angle shot (FLASH) MRI sequence parameters: 0.1×0.1×0.08 mm3 spatial resolution, 280×280 matrix size, 12.9 ms TR, 4.9 ms TE, 170 slices, 30° flip angle. (FIG. 14C) Electrophysiology signal recorded at the target coordinate in ZI during eNpHR experiments (highpass filtered, 300 Hz cutoff frequency, 4-pole Bessel filter). Neurons at the target coordinate are responsive to 4 s periods of contralateral, but not ipsilateral, whisker stimulation, consistent with known receptive field properties of zona incerta. Bottom trace shows a zoomed-in version of one contralateral whisker stimulation trial. (FIGS. 14D-14G) Histological and functional confirmation of halorhodopsin expression in zona incerta. (D) mCherry expression in zona incerta confirms expression of eNpHR-mCherry. (FIG. 14E) Recordings were performed in zona incerta during continuous 589 nm light delivery there to confirm functional halorhodopsin expression. (FIG. 14F) Perievent time histogram from a representative unit in zona incerta that exhibits a significant decrease in firing rate during eNpHR activation (p=1.3×10−5). A significant decrease in firing rate was observed in all recorded units (N=35 units, 20 trials). (FIG. 14G) Histogram of eNpHR-driven changes in firing rate across all recorded units in zona incerta (N=35 units). The average change in firing rate was −30%±15% s.t.d. (FIG. 14H) Recordings were performed in contralateral VLO during continuous suppression of zona incerta to investigate any tonic influence of ZI over cortex. (FIG. 14I) The majority of units recorded in contralateral VLO (92%) exhibited no significant change when zona incerta was inhibited with halorhodopsin. 8% exhibited a significant increase in activity.

FIG. 15 shows the amino acid sequences of depolarizing light-activated polypeptides and derivatives thereof (SEQ ID NOs:1-20) that may find use in the present methods, according to embodiments of the present disclosure.

FIG. 16 shows the amino acid sequences of hyperpolarizing light-activated polypeptides and derivatives thereof (SEQ ID NOs:21-51) that may find use in the present methods, according to embodiments of the present disclosure.

### DEFINITIONS

The terms “polypeptide”, “peptide” and “protein” are used interchangeably herein to refer to polymers of amino acids of any length. The polymer may be linear, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “genetic modification” refers to a permanent or transient genetic change induced in a cell following introduction into the cell of a heterologous nucleic acid (e.g., a nucleic acid exogenous to the cell). Genetic change (“modification”) can be accomplished by incorporation of the heterologous nucleic acid into the genome of the host cell, or by transient or stable maintenance of the heterologous nucleic acid as an extrachromosomal element. Where the cell is a eukaryotic cell, a permanent genetic change can be achieved by introduction of the nucleic acid into the genome of the cell.

Suitable methods of genetic modification include viral infection, transfection, conjugation, protoplast fusion, electroporation, particle gun technology, calcium phosphate precipitation, direct microinjection, and the like.

A “plurality” contains at least 2 members. In certain cases, a plurality may have at least 10, at least 100, at least 1000, at least 10,000, at least 100,000, at least 10⁶, at least 10⁷, at least 10⁸ or at least 10⁹ or more members.

“Substantially” as used herein, may be applied to modify any quantitative representation that could permissibly vary without resulting in a change in the basic function to which it is related.

An “individual” as used herein, may be any suitable animal amenable to the methods and techniques described herein, where in some cases, the individual may be a vertebrate animal, including a mammal, bird, reptile, amphibian, etc. The individual may be any suitable mammal, e.g., human, mouse, rat, cat, dog, pig, horse, cow, monkey, non-human primate, etc.

A “set”, as used herein, may include one or more elements.

“Functional”, as used herein, may be used to describe a process that is physiologically relevant, i.e., relevant for carrying out a process that normally occurs in a living organism. The process may be a measured phenomenon that is representative of, or a direct or indirect read out of, an underlying, physiologically relevant process.

A “connection” as used herein, may refer to a structural and/or functional relationship between two distinct entities, e.g., cells (including neurons), regions of a tissue (such as regions of a brain), tissues, organs, etc. A functional connection between two regions of the brain may be achieved by direct and/or indirect structural connections (e.g., synaptic connections) between the two regions.

“Neural activity” as used herein, may refer to electrical activity of a neuron (e.g., changes in membrane potential of the neuron), as well as indirect measures of the electrical activity of one or more neurons. Thus, neural activity may refer to changes in field potential, changes in intracellular ion concentration (e.g., intracellular calcium concentration), and changes in magnetic resonance induced by electrical activity of neurons, as measured by, e.g., cerebral blood volume (CBV) in functional magnetic resonance imaging.

“Dynamic” as used herein, may be applied to describe a process that varies in the temporal dimension.

“Quantitative” as used herein, refers to a numerical property defined by or is related to magnitude, or to describe a system (e.g., brain circuit) whose output varies with different patterns of input.

“Qualitative” as used herein, may refer to a property that is not defined by the magnitude of a numerical quantity. For instance, a qualitative determination may include determinations in which a yes/no or on/off result is determined.

In certain aspects, the term “modulating” means increasing, reducing or inhibiting. In some cases, “modulate” or “modulating” or “modulation” may be measured using an appropriate in vitro assay, cellular assay, in vivo assay, or behavioral assay. In some cases, the increase or decrease is 10% or more relative to a reference, e.g., 10% or more, 20% or more, 30% or more, 40% or more, 50% or more, 60% or more, 70% or more, 80% or more, 90% or more, 95% or more, 97% or more, 98% or more, up to 100% relative to a reference. For example, the increase or decrease may be 2 or more times, 3 times or more, 4 times or more, 5 times or more, 6 times or more, 7 times or more, 8 times or more, 9 times or more, 10 times or more, 50 times or more, or 100 times or more relative to a reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a neuron” includes a plurality of such neurons and reference to “the light-activated polypeptide” includes reference to one or more light-activated polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### DETAILED DESCRIPTION

Provided herein are methods and systems for modulating temporal patterns of neuronal activity in the brain. A method of the present disclosure may include using optogenetics to stimulate a one or more of thalamocortical projections, thalamic relay neurons, cortical projection neurons, cell bodies in a thalamic submedial nucleus, and cell bodies in the VLO in the brain, in conjunction with fMRI of different regions of the brain to directly visualize the global influence of the VLO's afferent and efferent connections, and characterize how different temporal patterns of activity in the VLO circuit affect brain dynamics by driving its input and output at distinct frequencies.

### Methods

As summarized above, methods are provided for modulating temporal patterns of neuronal activity in the brain of an individual. In some cases, the methods modulate neuronal activity in one or more brain regions or in the whole brain. In some cases, the methods modulate the spatial extent of neuronal activation or inhibition in one or more brain regions or in the whole brain. In some cases, the methods modulate the inhibitory or activating effects of inputs from one or more brain regions on one or more downstream brain regions. Aspects of the methods may include visualizing and/or measuring the neuronal activity, e.g., the temporal and/or spatial patterns of neuronal activity, in one or more brain regions or in the whole brain in response to stimulation of one or more brain regions. Methods of the present disclosure may use any number of combinations of suitable neuronal stimulation and neuronal activity measurement protocols, as necessary, to determine the functional connections between different brain regions. Suitable protocols include electrophysiology; light-induced modulation of neural activity; electroencephalography (EEG) recordings; functional imaging and behavioral analysis. One or more parameters, e.g., light pulse frequency, of the neuronal stimulation protocols may be varied. The one or more parameters may be varied to modulate neuronal activity as described herein. The neuronal stimulation and neuronal activity measurement protocols may be applied to the whole brain. The neuronal stimulation and neuronal activity measurement protocols may be applied to one or more brain regions. In some instances, the whole-brain includes an ipsilateral brain region and a contralateral brain region.

As summarized above, the methods may include any number of combinations of neuronal stimulation and neuronal activity measurement protocols. Some protocols, such as fMRI, provide a non-invasive, brain-wide measure representative of neural activity. Some protocols, such as electrophysiology, provide cellular resolution and rapid measures of neural activity as well as cellular resolution and rapid control of neural activity. Some protocols, such as optogenetics, provide spatially-targeted and temporally-defined control of action potential firing in defined groups of neurons. An appropriate combination of assays may be used to dissect a functional brain circuit. In some cases, the combination includes: optogenetics and fMRI; optogenetics and electrophysiology; optogenetics and EEG; optogenetics and behavioral analysis. Any other suitable combination, e.g., EEG and behavioral analysis; fMRI and electrophysiology; electrophysiology and behavioral analysis, etc., may also be used.

The methods disclosed herein are amenable to revealing causal links between different brain regions in a single living individual (e.g., a single mouse or rat, a single human, a single non-human mammal) by using one or more different combinations of neuronal stimulation and activity measurement protocols, as described above. In some instances, the methods identify circuit mechanisms underlying the control of brain-wide neural activities by one or more regions of the brain. Thus, in some embodiments, a brain functional circuit is assayed in a single animal using one or more combinations of optogenetics and fMRI; optogenetics and electrophysiology; optogenetics and EEG; and optogenetics and behavioral analysis. In some cases, a brain functional circuit is assayed in a single animal using all of optogenetics and fMRI; optogenetics and electrophysiology; optogenetics and EEG; and optogenetics and behavioral analysis. The order in which the different combinations of assays are performed on a single animal may be any suitable order. In some cases, the combinations of assays are performed in the order of: optogenetics and fMRI; optogenetics and EEG/optogenetics and behavioral analysis; and optogenetics and electrophysiology, where the pairs “optogenetics and EEG” and “optogenetics and behavioral” may be performed in any order. Other combinations of protocols may be performed at any suitable point before or after any of the combinations of protocols with optogenetics.

Aspects of the present disclosure may include methods of modulating temporal patterns of neuronal activity in the brain of an individual, using a combination of optogenetic stimulation of a defined set of neurons in one or more brain regions of the individual, and measuring the response at a whole-brain level by scanning the brain with fMRI, to modulate the neuronal activity following stimulation. Embodiments of the methods may include modulating temporal patterns of neuronal activity in the brain of an individual, using a combination of optogenetic stimulation of a defined set of neurons in one or more of the VLO and a thalamus of the individual, and measuring the response at a whole-brain level by scanning the brain with fMRI, to modulate the neuronal activity following stimulation.

The brain regions of interest in the present methods (for optogenetically stimulating and/or measuring neural activity) may vary and may be any suitable region. In certain embodiments, the brain regions are anatomically and/or functionally defined regions of the brain. For example, the first region of the brain and the second region of the brain illuminated by light pulses as described herein may be anatomically distinct regions of the brain. In some cases, where the brain is a mammalian brain, the brain region of interest is selected from at least a portion of the thalamus (including the central thalamus), sensory cortex (including the somatosensory cortex), zona incerta (ZI), ventral tegmental area (VTA), prefontal cortex (PFC), nucleus accumbens (NAc), amygdala (BLA), substantia nigra, ventral pallidum, globus pallidus, dorsal striatum, ventral striatum, subthalamic nucleus, hippocampus, dentate gyrus, cingulate gyrus, entorhinal cortex, olfactory cortex, primary motor cortex, and cerebellum. In some cases, different brain regions (e.g., the first and second brain regions) are separated at minimum by one or more, e.g., 2 or more, 3 or more, 4 or more, 5 or more, including 7 or more synaptic connections, and are separated at minimum by 15 or fewer, e.g., 12 or fewer, 10 or fewer, 8 or fewer, including 6 or fewer synaptic connections. In some embodiments, the different brain regions are separated at minimum by 1 to 15 synaptic connections, e.g., 1 to 12 synaptic connections, 1 to 10 synaptic connections, 2 to 8 synaptic connections, including 3 to 6 synaptic connections.

Neurons of interest and that are present in the brain regions may be any suitable types of neurons. In some cases, the neurons are inhibitory neurons, or excitatory neurons. In some cases, the neurons are sensory neurons, interneurons, or motor neurons. In some cases, the neurons are, without limitation, dopaminergic, cholinergic, GABAergic, glutamatergic, or peptidergic neurons.

In some cases, the methods of the present disclosure include stimulating the VLO of the brain. In some cases, the methods of the present disclosure include stimulating the thalamocortical projections of the brain. In some cases, the methods of the present disclosure include stimulating the thalamic relay neurons of the brain. In some cases, the methods of the present disclosure include stimulating the cortical projection neurons of the brain. In some cases, the methods of the present disclosure include stimulating the cell bodies in the thalamic submedial nucleus of the brain. In some cases, the methods of the present disclosure include stimulating the cell bodies in the VLO of the brain. In some cases, stimulating the VLO of the brain results in a positive measured fMRI signal at the VLO of the brain.

In practicing embodiments of the subject methods, the methods may include, e.g., i) stimulating, with a light pulse from an optical light source, one or more of thalamocortical projections, thalamic relay neurons, cortical projection neurons, cell bodies in a thalamic submedial nucleus, and cell bodies in the ventrolateral orbitofrontal cortex (VLO) in the brain, wherein neuronal cell bodies in one or more of the VLO and a thalamus of the individual express a light-activated polypeptide; and ii) measuring a functional magnetic resonance imaging (fMRI) signal of the whole-brain, wherein said measuring occurs during said stimulating, wherein a positive measured fMRI signal is associated with an increase in neuronal activity following said stimulating, and wherein a negative measured fMRI signal is associated with a decrease in neuronal activity following said stimulating.

Stimulation

The neurons in the one or more brain regions subjected to optogenetic stimulation may be modified to contain a light-activated polypeptide. The modification may occur by administering, e.g., injecting, a light-activated polypeptide to the one or more brain regions. Thus, the neurons in the VLO and/or thalamus may be modified to contain a light-activated polypeptide, e.g., a light-activated ion channel, where the light-activated polypeptide is configured to modulate the activity of, e.g., depolarize or hyperpolarize, the one or more neurons upon stimulating one or more thalamocortical projections, thalamic relay neurons, cortical projection neurons, cell bodies in a thalamic submedial nucleus, and cell bodies in the VLO in the brain with a light stimulus of appropriate wavelength, illumination volume and intensity. In some cases, the method includes expressing the light-activated polypeptide in neurons of the thalamus. In some cases, the method includes expressing the light-activated polypeptide in neurons of the submedial nucleus of the thalamus. In some cases, the method includes expressing the light-activated polypeptide in the neurons of the VLO. In some cases, the method includes expressing the light-activated polypeptide in the layer I and/or layer III neurons. In some cases, the light-activated polypeptide expressed in layer I and/or layer III neurons of the VLO comes from neurons that are located in the submedial nucleus of the thalamus. For example, the neurons of the submedial nucleus expressing the light-activated polypeptide send projections to the VLO. In some cases, the light-activated polypeptide is a depolarizing light-activated polypeptide. In some cases, the light-activated polypeptide is a hyperpolarizing light-activated polypeptide. In some embodiments, neurons in the submedial nucleus are modulated by stimulating cell bodies in the submedial nucleus. In some embodiments, neurons in the submedial nucleus are modulated by stimulating cell bodies in the projections in the VLO.

In some cases, the methods of the present disclosure include genetically modifying the neurons of the VLO and/or thalamus, e.g., by viral infection of a DNA construct containing nucleotide sequences encoding the light-activated polypeptide and any other appropriate regulatory elements, to express the light-activated polypeptide. In some instances, the methods include administering a light-activated polypeptide to the submedial nucleus of the thalamaus. Any suitable light-activated polypeptide may be used, as described further herein. In some cases, the methods of the present disclosure include a first light-activated polypeptide and a second light-activated polypeptide. In some cases, a first light-activated polypeptide is a depolarizing light-activated polypeptide. In some cases, a second light-activated polypeptide is a hyperpolarizing light-activated polypeptide. In some cases, the methods of the present disclosure include administering the first and the second light-activated polypeptides in the same region of the brain. In some cases, the methods of the present disclosure include administering the first and the second light-activated polypeptides in the different regions of the brain. Suitable light-activated polypeptides are described in U.S. Patent Publication No. 2018/0360343A1, which is hereby incorporated by reference in its entirety.

Aspects of the present methods may include administering a second light-activated polypeptide. In some cases, the second light-activated polypeptide is administered into the zona incerta (ZI) region of the brain. In some cases, the second light-activated polypeptide is a depolarizing light-activated polypeptide. In some cases, the second light-activated polypeptide is a hyperpolarizing light-activated polypeptide. In some cases, the methods of the present disclosure include stimulating the ZI region of the brain, for example, when a second light-activated polypeptide is expressed in neurons of the ZI. The ZI region may be stimulated simultaneously during stimulation of other brain regions and/or performing of electrophysiological recordings. The ZI region may be stimulated simultaneously during stimulation of thalamocortical projections. The ZI region may be stimulated with light pulses having any frequency as described herein.

Neurons of a suitable region of the brain whose activity is to be modulated by light can be modified using a convenient method to express the light-activated polypeptide. In some cases, neurons of a brain region are genetically modified to express a light-activated polypeptide. In some cases, the neurons may be genetically modified using a viral vector, e.g., an adeno-associated viral vector, containing a nucleic acid having a nucleotide sequence that encodes the light-activated polypeptide. The viral vector may include any suitable control elements (e.g., promoters, enhancers, recombination sites, etc.) to control expression of the light-activated polypeptide according to cell type, timing, presence of an inducer, etc.

Suitable neuron-specific control sequences include, but are not limited to, a neuron-specific enolase (NSE) promoter (see, e.g., EMBL HSENO2, X51956; see also, e.g., U.S. Pat. Nos. 6,649,811, 5,387,742); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn et al. (2010) Nat. Med. 16:1161); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; a CMV enhancer/platelet-derived growth factor-β promoter (see, e.g., Liu et al. (2004) Gene Therapy 11:52-60); a motor neuron-specific gene Hb9 promoter (see, e.g., U.S. Pat. No. 7,632,679; and Lee et al. (2004) Development 131:3295-3306); and an alpha subunit of Ca(²⁺)-calmodulin-dependent protein kinase II (CaMKIIα) promoter (see, e.g., Mayford et al. (1996) Proc. Natl. Acad. Sci. USA 93:13250). Other suitable promoters include elongation factor (EF) 1α and dopamine transporter (DAT) promoters.

In some cases, cell type-specific expression of the light-activated polypeptide may be achieved by using recombination systems, e.g., Cre-Lox recombination, Flp-FRT recombination, etc. Cell type-specific expression of genes using recombination has been described in, e.g., Fenno et al., Nat Methods. 2014 July; 11(7):763; and Gompf et al., Front Behav Neurosci. 2015 Jul. 2; 9:152, which are incorporated by reference in their entirety.

A light stimulus may be used to illuminate the one or more brain regions containing the light-activated polypeptide. The light stimulus may be used to activate the one or more light-activated polypeptides. The light stimulus used to activate the light-activated polypeptide may include one or more light pulses. The light pulses may be characterized by, e.g., frequency, pulse width, duty cycle, wavelength, intensity, etc. In some cases, the light stimulus includes two or more different sets of light pulses, where each set of light pulses is characterized by different temporal patterns of light pulses. The temporal pattern may be characterized by any suitable parameter, including, but not limited to, frequency, period (i.e., total duration of the light stimulus), pulse width, duty cycle, etc. Optogenetic stimulation may be performed using any suitable method. Suitable methods are described in, e.g., U.S. Pat. No. 8,834,546, which is hereby incorporated by reference in its entirety.

The variation in the property of the light pulses of a set may be reflected in a difference in the activity of the illuminated neurons. In some cases, where the neuron is depolarized by activation of the light-activated polypeptide, an increase in the frequency of the light pulses may cause an increase in the frequency of action potential firing in the illuminated neurons. In some embodiments, the frequency of action potential firing in the illuminated neurons scales quantitatively with the increase in the frequency of the light pulses. In some cases, a linear increase in the frequency of the light pulses may induce a linear, or non-linear but monotonic, increase in the frequency of action potential firing in the illuminated neurons. In some instances, stimulation may be manifested as downregulation of neuronal activity, e.g., neuronal hyperpolarization. In some cases, where the neuron is hyperpolarized by activation of the light-activated polypeptide, an increase in the frequency of the light pulses may cause a decrease in the frequency of action potential firing in the illuminated neurons. Aspects of the present disclosure may include stimulating or illuminating a first region of the brain with a first set of light pulses and a second set of light pulses that have a different temporal pattern, where neurons in the first region may generate action potentials induced by the first set and/or second set of light pulses, or inhibit action potentials following the first set and/or second set of light pulses.

In some cases, the light stimulus contains one or more, two or more, three or more, four or more, five or more, six or more, seven of more, eight or more, nine or more, or ten or more sets of light pulses, where the sets of light pulses are characterized by having different parameter values, such as different frequencies of light pulses. Where the sets of light pulses have different frequencies, the duty cycle may be the same, or may be different. In some cases, the sets of light pulses with different frequencies have the same pulse width. In other instances, the sets of light pulses with different frequencies have different pulse widths.

The light pulses of a set may have any suitable frequency. In some cases, the set of light pulses contains a single pulse of light that is sustained throughout the duration of the light stimulus. In some cases, the light pulses of a set have a frequency of 0.1 Hz or more, e.g., 0.5 Hz or more, 1 Hz or more, 5 Hz or more, 10 Hz or more, 20 Hz or more, 30 Hz or more, 40 Hz or more, including 50 Hz or more, or 60 Hz or more, or 70 Hz or more, or 80 Hz or more, or 90 Hz or more, or 100 Hz or more, and have a frequency of 100,000 Hz or less, e.g., 10,000 Hz or less, 1,000 Hz or less, 500 Hz or less, 400 Hz or less, 300 Hz or less, 200 Hz or less, including 100 Hz or less. In some cases, the light pulses of a set have a frequency in the range of 0.1 to 100,000 Hz, e.g., 1 to 10,000 Hz, 1 to 1,000 Hz, including 5 to 500 Hz, or 10 to 100 Hz. In some embodiments, the light pulse has a frequency ranging from 5 Hz to 40 Hz.

The light pulses of the present methods may have any suitable pulse width. In some cases, the pulse width is 0.1 ms or longer, e.g., 0.5 ms or longer, 1 ms or longer, 3 ms or longer, 5 ms or longer, 7.5 ms or longer, 10 ms or longer, including 15 ms or longer, or 20 ms or longer, or 25 ms or longer, or 30 ms or longer, or 35 ms or longer, or 40 ms or longer, or 45 ms or longer, or 50 ms or longer, and is 500 ms or shorter, e.g., 100 ms or shorter, 90 ms or shorter, 80 ms or shorter, 70 ms or shorter, 60 ms or shorter, 50 ms or shorter, 45 ms or shorter, 40 ms or shorter, 35 ms or shorter, 30 ms or shorter, 25 ms or shorter, including 20 ms or shorter. In some embodiments, the pulse width is in the range of 0.1 to 500 ms, e.g., 0.5 to 100 ms, 1 to 80 ms, including 1 to 60 ms, or 1 to 50 ms, or 1 to 30 ms.

The duty cycle of the pulses of the present methods may be any suitable duty cycle. In some cases, the duty cycle is 1% or more, e.g., 5% or more, 10% or more, 15% or more, 20% or more including 25% or more, or 30% or more, or 35% or more, or 40% or more, or 45% or more, or 50% or more, and may be 80% or less, e.g., 75% or less, 70% or less, 65% or less, 60% or less, 65% or less, 50% or less, 45% or less, including 40% or less, or 35% or less, or 30% or less. In certain embodiments, the duty cycle is in the range of 1 to 80%, e.g., 5 to 70%, 5 to 60%, including 10 to 50%, or 10 to 40%.

The average power of the light pulse of the present methods, measured at the tip of an optical fiber delivering the light pulse to regions of the brain, may be any suitable power. In some cases, the power is 0.1 mW or more, e.g., 0.5 mW or more, 1 mW or more, 1.5 mW or more, including 2 mW or more, or 2.5 mW or more, or 3 mW or more, or 3.5 mW or more, or 4 mW or more, or 4.5 mW or more, or 5 mW or more, and may be 1,000 mW or less, e.g., 500 mW or less, 250 mW or less, 100 mW or less, 50 mW or less, 40 mW or less, 30 mW or less, 20 mW or less, 15 mW or less, including 10 mW or less, or 5 mW or less. In some embodiments, the power is in the range of 0.1 to 1,000 mW, e.g., 0.5 to 100 mW, 0.5 to 50 mW, 1 to 20 mW, including 1 to 10 mW, or 1 to 5 mW.

The wavelength and intensity of the light pulses of the present methods may vary and may depend on the activation wavelength of the light-activated polypeptide, optical transparency of the region of the brain, the desired volume of the brain to be illuminated, etc.

The volume of a brain region illuminated by the light pulses may be any suitable volume. In some cases, the illuminated volume is 0.001 mm³ or more, e.g., 0.005 mm³ or more, 0.001 mm³ or more, 0.005 mm³ or more, 0.01 mm³ or more, 0.05 mm³ or more, including 0.1 mm³ or more, and is 100 mm³ or less, e.g., 50 mm³ or less, 20 mm³ or less, 10 mm³ or less, 5 mm³ or less, 1 mm³ or less, including 0.1 mm³ or less. In certain cases, the illuminated volume is in the range of 0.001 to 100 mm³, e.g., 0.005 to 20 mm³, 0.01 to 10 mm³, 0.01 to 5 mm³, including 0.05 to 1 mm³.

In some cases, the methods of the present disclosure include reversibly inserting an optical light source, e.g., an optical fiber, in the VLO of the individual. In some cases, the optical light source is implanted. In some cases, the optical light source, e.g., optical fiber, is removably inserted and/or implanted in the VLO. In some cases, the optical light source is removable. In some cases, the regions of the brain with neurons containing a light-activated polypeptide, is stimulated or illuminated using an optical light source comprising one or more optical fibers. In some cases, the optical fiber is coupled with a laser source. The optical fiber may be configured in any suitable manner to direct a light emitted from suitable source of light, e.g., a laser or light-emitting diode (LED) light source, to the region of the brain.

Aspects of the present disclosure further include methods of modulating pain in an individual. In some cases, the method includes i) stimulating one or more of thalamocortical projections, thalamic relay neurons, cortical projection neurons, cell bodies in a thalamic submedial nucleus, and cell bodies in the VLO in the brain of the individual with one or more light pulses, wherein neuronal cell bodies in one or more of the VLO and a thalamus of an individual expresses a light-activated polypeptide, and wherein said stimulation modulates pain in an individual. Modulating pain in the individual may include, e.g., modulating the neuronal activity in response to noxious stimuli or modulating neuronal activity associated with aversive or painful sensations in the orbitofrontal cortex of the brain.

In some cases, stimulating one or more of thalamocortical projections, thalamic relay neurons, cortical projection neurons, cell bodies in a thalamic submedial nucleus, and cell bodies in the VLO in the brain with a first set of light pulses inhibits the neuronal activity in response to noxious stimuli. Noxious stimuli may include chemical, thermal, and/or mechanical stimuli. In some cases, the noxious stimuli include, e.g., heat, one or more chemicals, and irradiation.

In some cases, stimulating one or more of thalamocortical projections, thalamic relay neurons, cortical projection neurons, cell bodies in a thalamic submedial nucleus, and cell bodies in the VLO in the brain with a first set of light pulses inhibits the neuronal activity associated with aversive or painful sensations in the orbitofrontal cortex of the brain.

In some cases, stimulating one or more of thalamocortical projections, thalamic relay neurons, cortical projection neurons, cell bodies in a thalamic submedial nucleus, and cell bodies in the VLO in the brain with a second set of light pulses activates the neuronal activity associated with aversive or painful sensations in the orbitofrontal cortex of the brain.

### Response to Stimulation

The responses to the stimulation by different sets of light pulses may be measured by any suitable brain imaging or neuronal activity measurement protocol, e.g., fMRI, for the whole brain. A comparison of the responses in each region of the brain may indicate a functional connection between neurons stimulated by the light stimulus to the one or more of thalamocortical projections, thalamic relay neurons, cortical projection neurons, cell bodies in a thalamic submedial nucleus, and cell bodies in the VLO and other regions of the brain, such as on thalamic brain regions downstream from their projection site. In some cases, a quantitative change in the light pulse may cause a change in sign of the fMRI cerebral blood volume (CBV) (e.g., a positive or negative CBV response is measured depending on the frequency of the light pulses).

In some cases, the methods of the present disclosure include measuring a fMRI signal of the whole-brain during stimulation of the one or more of thalamocortical projections, thalamic relay neurons, cortical projection neurons, cell bodies in a thalamic submedial nucleus, and cell bodies in the VLO in the brain. In some cases, fMRI signals are measured in the ipsilateral region, which includes a left hemisphere of the brain comprising a medial prefontal cortex, a lateral prefontal cortex, a motor cortex, a cingulate cortex, a sensory cortex, an insular cortex, a striatum, and a thalamus. In some cases, the method includes measuring fMRI signals in the contralateral region of the brain, which includes a right hemisphere of the brain comprising a medial prefontal cortex, a lateral prefontal cortex, a motor cortex, a cingulate cortex, a sensory cortex, an insular cortex, a striatum, and a thalamus. In some instances, measuring the fMRI signal includes measuring a cerebral blood volume.

In certain embodiments, fMRI may be used to indirectly measure neuronal activity in one or more regions of the brain. For example, fMRI can be used to indirectly measure neuronal activity in different regions of the brain before, during, or after stimulating or illuminating, e.g., with an optical fiber, a first region of the brain with a first set of light pulses and a second set of light pulses that have a different temporal pattern, where neurons in the first region may generate action potentials induced by the first set and/or second set of light pulses, or inhibit action potentials following the first set and/or second set of light pulses. In some cases, an increase in neural activity induced by a set of light pulses, e.g., the first set of light pulses, in a region of the brain as provided herein can be associated with a measured fMRI signal. In addition, a decrease in neural activity induced by a set of light pulses, e.g., the second set of light pulses, in a region of the brain as provided herein can also be associated with a measured fMRI signal. In some cases, a negative measured fMRI signal is associated with a decrease in neuronal activity induced by a set of light pulses in one or more brain regions. In some cases, a positive measured fMRI signal is associated with an increase in neuronal activity induced by a set of light pulses in one or more brain regions. In some cases, a negative measured fMRI signal is associated with a decrease in neuronal activity following stimulation to the one or more of thalamocortical projections, thalamic relay neurons, cortical projection neurons, cell bodies in a thalamic submedial nucleus, and cell bodies of the VLO. In some cases, a positive measured fMRI signal is associated with an increase in neuronal activity following stimulation to the one or more of thalamocortical projections, thalamic relay neurons, cortical projection neurons, cell bodies in a thalamic submedial nucleus, and cell bodies of the VLO.

The response to stimulation, e.g., as measured by fMRI, may be dependent on the frequency of the light pulses and/or the collection of neurons or brain region that is illuminated. For example, the frequency of the light pulse may determine whether the fMRI signal in one or more brain regions is positive or negative. In some cases, the light pulse is delivered at a frequency that results in a negative measured fMRI signal. In some cases, the light pulse is delivered at a frequency that results in a positive measured fMRI signal. In some instances, stimulating a first brain region with a light pulse results in a negative fMRI signal in one or more downstream brain regions, e.g., brain regions that receive input from the first brain region. In some instances, stimulating a first brain region with a light pulse results in a positive fMRI signal in one or more downstream brain regions.

In some cases, the light pulse has a frequency of 5 Hz or more. In some cases, stimulating thalamocortical projections with the pulse having a frequency of 5 Hz or more results in a negative measured fMRI signal. In some cases, the negative measured fMRI signal is in the sensory, motor, and cingulate cortex of the ipsilateral region of the brain. In some cases, stimulating thalamocortical projections with the pulse having a frequency of 5 Hz or more results in the negative measured fMRI in the contralateral region of the brain. In some cases, the negative measured fMRI signal is associated with a decrease in neuronal activity in the contralateral region of the brain. In some instances, stimulating the thalamocortical projections with the light pulse having a frequency of 5 Hz or more inhibits the neuronal activity of the ipsilateral thalamus of the brain.

In some cases, the light pulse has a frequency of 10 Hz or more. In some cases, stimulating thalamocortical projections with the pulse having a frequency of 10 Hz or more results in a negative measured fMRI signal. In some cases, the negative measured fMRI signal is in the sensory, motor, and cingulate cortex of the ipsilateral region of the brain. In some cases, stimulating thalamocortical projections with the pulse having a frequency of 10 Hz or more results in a negative measured fMRI signal. In some cases, the negative measured fMRI signal is in the sensory, motor, and cingulate cortex of the ipsilateral region of the brain. In some cases, the negative measured fMRI signal is associated with a decrease in neuronal activity in the sensory, motor, and cingulate cortex of the ipsilateral region of the brain. In some cases, stimulating the thalamocortical projections with the light pulse having a frequency of 10 Hz or more results in the negative measured fMRI signal in the contralateral region of the brain. In some cases, the negative measured fMRI signal is associated with a decrease in neuronal activity in the contralateral region of the brain. In some cases, stimulating the thalamocortical projections with the light pulse having a frequency of 10 Hz or more results in the negative measured fMRI signal in the cortex, contralateral striatum, and contralateral thalamus of the brain.

In some cases, the light pulse has a frequency ranging from 5 Hz to 20 Hz. In some cases, stimulating the thalamocortical projections at the light pulse having a frequency ranging from 5 Hz to 20 Hz results in a negative measured fMRI signal in the contralateral region of the brain. In some cases, stimulating the thalamocortical projections at the light pulse having a frequency ranging from 5 Hz to 20 Hz inhibits neuronal activity in the contralateral region of the brain. In some cases, the contralateral region comprises the prefrontal cortex of the brain. In some cases, the negative measured fMRI signal is associated with a decrease in neuronal activity in the contralateral region of the brain. In some cases, stimulating thalamocortical projections with the light pulse having a frequency ranging from 5 Hz or more, 10 Hz or more, 15 Hz or more, or 20 Hz inhibits the neuronal activity of the contralateral region of the brain.

In some cases, the light pulse has a frequency ranging from 20 Hz to 40 Hz. In some cases, stimulating thalamocortical projections with the pulse having a frequency ranging from 20 Hz to 40 Hz results in a positive measured fMRI signal. In some cases, the positive measured fMRI signal is associated with an increase in neuronal activity in the ipsilateral region of the brain. In some cases, the positive measured fMRI signal is associated with an increase in neuronal activity in the ipsilateral thalamus of the brain. In some cases, stimulating thalamocortical projections with the light pulse having a frequency ranging from 20 Hz or more, 25 Hz or more, 30 Hz or more, 35 Hz or more, or 40 Hz or more results in the positive measured fMRI signal in the ipsilateral region of the brain associated with an increase in neuronal activity in the ipsilateral region of the brain. In some instances, stimulating the thalamocortical projections with the light pulse having a frequency ranging from 20-40 Hz activates the neuronal activity of the ipsilateral thalamus of the brain. In some instances, stimulating thalamocortical projections with the light pulse having a frequency ranging from 25 Hz or more results in a negative measured fMRI signal in the contralateral region of the brain.

In some cases, the light pulse has a frequency of 40 Hz or more. In some cases, stimulating thalamocortical projections with the pulse having a frequency of 40 Hz or more results in a positive measured fMRI signal. In some cases, stimulation of thalamocortical projections with the light pulse with a frequency of 40 Hz or more results in a positive fMRI signal in the ipsilateral region of the brain. In some cases, the positive measured fMRI signal is associated with an increase in neuronal activity in the ipsilateral region of the brain. In some cases, the positive measured fMRI signal is associated with an increase in neuronal activity in the ipsilateral thalamus of the brain. In some instances, stimulation of thalamocortical projections with the light pulse with a frequency of 40 Hz or more results in a positive fMRI signal in the ipsilateral thalamus, ipsilateral striatum, and ipsilateral cortex of the brain.

In some cases, the light pulse has a frequency ranging from 5 Hz to 40 Hz. In some cases, stimulating the cell bodies in the VLO with the light pulse having a frequency ranging from 5 Hz to 40 Hz results in the positive measured fMRI signal in the ipsilateral region of the brain. In some cases, stimulating the cell bodies in the VLO with the light pulse having a frequency ranging from 5 Hz or more, 10 Hz or more, 15 Hz or more, 20 Hz or more, 25 Hz or more, 30 Hz or more, 35 Hz or more, or 40 Hz or more results in the positive measured fMRI signal in the ipsilateral region of the brain. In some cases, the positive measured fMRI signal is associated with an increase in neuronal activity in the ipsilateral thalamus of the brain. In some instances, stimulating cell bodies at the light pulse having a frequency of 40 Hz or more increases the neuronal activity of the ipsilateral thalamus of the brain.

In some cases, the light pulse has a frequency ranging from 5 Hz to 40 Hz. In some cases, stimulating the cell bodies of the thalamic submedial nucleus results in a positive measured fMRI signal in the ipsilateral thalamus of the brain. In some cases, stimulating the cell bodies of the thalamic submedial nucleus with the light pulse having a frequency ranging from 5 Hz or more, 10 Hz or more, 15 Hz or more, 20 Hz or more, 25 Hz or more, 30 Hz or more, 35 Hz or more, or 40 Hz or more results in the positive measured fMRI signal in the ipsilateral region of the brain. In some cases, the positive measured fMRI signal is associated with an increase in neuronal activity in the ipsilateral thalamus of the brain.

In some cases, the light pulse has a frequency ranging from 5 Hz to 10 Hz. In some instances, stimulation of thalamocortical projections with the light pulse having a frequency ranging from 5 Hz to 10 Hz decreases brain activity in the ipsilateral region of the brain. In some cases, stimulation with the light pulse having a frequency ranging from 5 Hz to 10 Hz inhibits the neuronal activity of the ipsilateral thalamus of the brain.

Aspects of the present methods include performing electrophysiological recordings to detect firing rates of neurons in one or more brain regions associated with a measured fMRI signal. In some instances, the electrophysiological recordings detect neuronal activity associated with a positive or negative fMRI signal. In some instances, a positive fMRI signal may reflect an increase in neuronal firing rates. In some instances, a negative fMRI signal may reflect a decrease in neuronal firing rates. In some instances, the electrophysiological recordings are performed at the site of stimulation. In some cases, the electrophysiological recordings are performed at a site in the brain downstream from the one or more brain regions subjected to stimulation. In some instances, the electrophysiological recordings are performed at the site associated with an fMRI signal, e.g., a positive or negative fMRI signal. In some instances, the electrophysiological recordings are used to detect firing rates in one or more brain regions during or after stimulation at one or more frequencies. In some cases, the electrophysiological recordings are performed in the VLO. In some cases, the electrophysiological recordings are performed in the ipsilateral region of the brain. In some cases, the electrophysiological recordings are performed in the contralateral region of the brain. In some cases, the electrophysiological recordings are performed in the thalamic reticular nucleus. In some case, the electrophysiological recordings are performed in the contralateral reticular nucleus. In some instances, the increase or decrease in the firing rate of neurons in one or more brain regions may be modulated by varying the frequencies of the light pulse used for stimulation. Electrophysiology may include single electrode, multi electrode, and/or field potential recordings.

In some cases, the methods include performing electrophysiological recordings in one or more brain regions comprising the ipsilateral VLO of the brain. In some cases, a positive measured fMRI signal is associated with an increased firing rate of neurons recorded in the ipsilateral VLO. In some cases, a negative measured fMRI signal is associated with a decreased firing rate of neurons recorded in the ipsilateral VLO. In some instances, the one or more brain regions is the ipsilateral motor cortex. In some cases, stimulating at the light pulse having a frequency of 10 Hz or more results in a decrease in firing rate of neurons in the ipsilateral motor cortex. In some cases, stimulating at the light pulse having a frequency of 40 Hz or more results in an increase in firing rate of neurons in the ipsilateral motor cortex.

In some cases, the methods include performing electrophysiological recordings in one or more brain regions comprising the contralateral VLO of the brain. In some cases, a negative measured fMRI signal is associated with a decreased firing rate of neurons in the contralateral VLO. In some cases, stimulating the contralateral VLO associated with the negative measured fMRI signal is associated with a decreased firing rate of neurons in the contralateral VLO. In some cases, stimulating the contralateral VLO at the light pulse having a frequency of 10 Hz or more results in a decreased firing rate of neurons in the contralateral VLO. In some cases, stimulating at the light pulse having a frequency of 40 Hz or more results in an increase in firing rate of neurons in the contralateral VLO.

### Systems

Aspects of the present disclosure include systems for carrying out the methods of the present disclosure in modulating temporal patterns of neuronal activity in the brain of an individual. In some cases, the systems modulate neuronal activity in one or more brain regions or in the whole brain. In some cases, the systems modulate the spatial extent of neuronal activation or inhibition in one or more brain regions or in the whole brain. In some cases, the systems modulate the inhibitory or activating effects of inputs from one or more brain regions on one or more downstream brain regions. Aspects of the systems may include a subsystem or device for visualizing and/or measuring the temporal and/or spatial patterns of neuronal activity in one or more brain regions or in the whole brain in response to stimulation of one or more brain regions. Systems of the present disclosure may use any number of combinations of suitable subsystems, apparatuses, or devices for stimulating neurons and measuring neuronal activity, as necessary, to determine the functional connections between different brain regions. Suitable subsystems, apparatuses, or devices include those used to perform electrophysiology recordings; light-induced modulation of neural activity; electroencephalography (EEG) recordings; functional imaging. In some instances, the whole-brain includes an ipsilateral and contralateral brain region.

The brain regions of interest in the present system (for optogenetically stimulating and/or measuring neural activity) may vary and may be any suitable region. In certain embodiments, the brain regions are anatomically and/or functionally defined regions of the brain. For example, the first region of the brain and the second region of the brain illuminated by light pulses as described herein may be anatomically distinct regions of the brain. In some cases, where the brain is a mammalian brain, the brain region of interest is selected from at least a portion of the thalamus (including the central thalamus), sensory cortex (including the somatosensory cortex), zona incerta (ZI), ventral tegmental area (VTA), prefontal cortex (PFC), nucleus accumbens (NAc), amygdala (BLA), substantia nigra, ventral pallidum, globus pallidus, dorsal striatum, ventral striatum, subthalamic nucleus, hippocampus, dentate gyrus, cingulate gyrus, entorhinal cortex, olfactory cortex, primary motor cortex, and cerebellum. In some cases, different brain regions (e.g., the first and second brain regions) are separated at minimum by one or more, e.g., 2 or more, 3 or more, 4 or more, 5 or more, including 7 or more synaptic connections, and are separated at minimum by 15 or fewer, e.g., 12 or fewer, 10 or fewer, 8 or fewer, including 6 or fewer synaptic connections. In some embodiments, the different brain regions are separated at minimum by 1 to 15 synaptic connections, e.g., 1 to 12 synaptic connections, 1 to 10 synaptic connections, 2 to 8 synaptic connections, including 3 to 6 synaptic connections.

Neurons of interest and that are present in the brain regions may be any suitable types of neurons. In some cases, the neurons are inhibitory neurons, or excitatory neurons. In some cases, the neurons are sensory neurons, interneurons, or motor neurons. In some cases, the neurons are, without limitation, dopaminergic, cholinergic, GABAergic, glutamatergic, or peptidergic neurons.

In some cases, the system of the present disclosure includes an optical source for stimulating the VLO of the brain. In some cases, the thalamocortical projections of the brain is stimulated. In some cases, the thalamic relay neurons of the brain is stimulated. In some cases, the cortical projection neurons of the brain are stimulated. In some cases, the cell bodies in the thalamic submedial nucleus of the brain are stimulated. In some cases, the cell bodies in the VLO of the brain are stimulated. In some cases, stimulation of the VLO of the brain results in a positive measured fMRI signal at the VLO of the brain.

In practicing embodiments of the methods, the systems of the present disclosure may include, e.g., i) a light source configured to stimulate, with a light pulse, one or more of thalamocortical projections, thalamic relay neurons, cortical projection neurons, cell bodies in a thalamic submedial nucleus, and cell bodies in the VLO in the brain of the individual, wherein a light-responsive opsin polypeptide is expressed in cell bodies in one or more of a VLO and a thalamus of the brain; and ii) a fMRI device configured to scan the whole-brain during stimulation to produce an fMRI signal; wherein a positive measured fMRI signal is associated with an increase in neuronal activity following stimulation, and wherein a negative measured fMRI signal is associated with a decrease in neuronal activity following stimulation. Embodiments of the present system may further include an electrophysiological recording device to record and detect firing rates of neurons in one or more brain regions associated with a measured fMRI signal.

As summarized above, aspects of the present disclosure include a system of modulating temporal patterns of neuronal activity in the brain of an individual, using a combination of optogenetic stimulation of a defined set of neurons in one or more of the VLO and a thalamus of the individual, and an fMRI device for measuring the response at a whole-brain level by scanning the brain with fMRI, to modulate the neuronal activity following stimulation. Thus, the neurons in the in the VLO and/or thalamus may be modified to contain a light-activated polypeptide, e.g., a light-activated ion channel, where the light-activated polypeptide is configured to modulate the activity of, e.g., depolarize or hyperpolarize, the neuron upon stimulation of one or more thalamocortical projections, thalamic relay neurons, cortical projection neurons, cell bodies in a thalamic submedial nucleus, and cell bodies in the VLO in the brain with a light stimulus of appropriate wavelength, illumination volume and intensity. In some cases, the neurons of the thalamus express the light-activated polypeptide. In some cases, the neurons of submedial nucleus of the thalamus express the light-activated polypeptide. In some cases, the neurons of the VLO express the light-activated polypeptide. In some cases, the neurons of the VLO that express the light-activated polypeptide are layer I and/or layer III neurons of the VLO. In some cases, the light-activated polypeptide expressed in layer I and/or layer III neurons of the VLO comes from neurons that are located in the submedial nucleus of the thalamus. For example, the neurons of the submedial nucleus expressing the light-activated polypeptide send projections to the VLO. In some cases, the light-activated polypeptide is a depolarizing light-activated polypeptide. In some cases, the light-activated polypeptide is a hyperpolarizing light-activated polypeptide. In some embodiments, neurons in the submedial nucleus are modulated by stimulation of cell bodies in the submedial nucleus. In some embodiments, neurons in the submedial nucleus are modulated by stimulation of cell bodies in the projections in the VLO.

In some cases, the neurons of the VLO and/or thalamus are genetically modified, e.g., by viral infection of a DNA construct containing nucleotide sequences encoding the light-activated polypeptide and any other appropriate regulatory elements, to express the light-activated polypeptide. Any suitable light-activated polypeptide may be used, as described further herein. In some cases, the methods of the present disclosure include a first light-activated polypeptide and a second light-activated polypeptide. In some cases, a first light-activated polypeptide is a depolarizing light-activated polypeptide. In some cases, a second light-activated polypeptide is a hyperpolarizing light-activated polypeptide. In some cases, the methods of the present disclosure include administering the first and the second light-activated polypeptides in the same region of the brain. In some cases, the methods of the present disclosure include administering the first and the second light-activated polypeptides in the different regions of the brain. Suitable light-activated polypeptides are described in U.S. Patent Publication No. 2018/0360343A1, which is hereby incorporated by reference in its entirety.

The systems may include an optical light source. The optical light source may be operatively coupled to an illumination unit, including one or more light sources, e.g., a light-emitting diode (LED) and/or a laser light source, that may be configured to emit light at a suitable wavelength. Having multiple light sources can allow the user to control the illumination pattern, e.g., the timing of light pulses, for each light source independently of each other. The illumination unit may also include any other suitable optical components to direct, focus and otherwise control the light being generated by the light source. Suitable optical components include, but are not limited to, lenses, tube lenses, collimators, dichroic mirrors, filters, shutters, etc. Thus, in certain embodiments, the illumination unit may be configured to project a light stimulus that includes light pulses of a number of wavelengths. A controller may be in communication with the illumination unit so as to control the timing, duration, and/or wavelength of the light pulse generated by the illumination unit. The systems may further include a power supply.

The light source of a system of the present disclosure may include any suitable light source. In some cases, the light source is an LED, an LED array or a laser. The light source may emit light having a wavelength in the infrared range, near-infrared range, visible range, and/or ultra-violet range. The light source may emit a light at a wavelength around 350 nm or more, e.g., around 380 nm or more, around 410 nm or more, around 440 nm or more, around 470 nm or more, around 500 nm or more, around 560 nm or more, around 594 nm or more, around 600 nm or more, around 620 nm or more, around 650 nm or more, around 680 nm or more, around 700 nm or more, around 750 nm or more, around 800 nm or more, including around 900 nm or more, and may emit a light at a wavelength around 2,000 nm or less, e.g., around 1,500 nm or less, 1,000 nm or less, 800 nm or less, 700 nm or less, 650 nm or less, including 620 nm or less, or 600 nm or less. In some cases, the light source may emit a light at a wavelength in the range of about 350 nm to about 2,000 nm, e.g., about 410 nm to about 2,000 nm, about 440 nm to about 1,000 nm, about 440 nm to about 800 nm, including about 440 nm to about 620 nm. The light source may be configured to produce a continuous wave, a quasi-continuous wave, or a pulsed wave light beam. In certain embodiments, a laser light source is a gas laser, solid state laser, a dye laser, semiconductor laser (e.g., a diode laser), or a fiber laser.

The number of wavelengths produced by the light source may be any suitable number of wavelengths. In some cases, the light source produces light with 1 or more, e.g., 2 or more, 3 or more, including 4 or more, or 5 or more, or 6 or more, or 7 or more, or 8 or more, or 9 or more, or 10 or more, distinct wavelengths of light, and produces light with 10 or fewer, e.g., 9 or fewer, 8 or fewer, 7 or less, 6 or fewer, including 5 or fewer distinct wavelengths of light. In some embodiments, the light source produces light in the range of 1 to 10, e.g., 1 to 8, 2 to 6, 2 to 5, including 2 to 4 distinct wavelengths.

In some cases, the systems of the present disclosure include an optical light source that can be reversibly inserted in the brain of the individual, such as, for example, in the VLO of the individual. In some cases, the optical light source is implanted. In some cases, the optical light source is removable. In some cases, the regions of the brain with neurons containing a light-activated polypeptide, is stimulated or illuminated using an optical light source comprising one or more optical fibers. In some cases, the optical fiber is coupled with a laser source. The optical fiber may be configured in any suitable manner to direct a light emitted from suitable source of light, e.g., a laser or light-emitting diode (LED) light source, to the region of the brain.

In some instances, the optical light source can be reversibly inserted in one or more regions of the brain of an individual. In some instances, the optical light source can be reversibly inserted in the VLO of the individual. In certain embodiments, the optical light source can be implanted in a region of the brain. In some cases, the optical light source is configured to deliver light to a targeted tissue structure after implantation in a location adjacent to the targeted tissue structure. In certain embodiments, the optical light source may be implanted in a dorsal position in the VLO of the brain.

In some cases, the optical light source is an optical fiber. The optical fiber may be any suitable optical fiber. In some cases, the optical fiber is a multimode optical fiber. In some instances, a multimode optical fiber supports more than one propagation mode. For example, a multimode optical fiber may be configured to carry a range of wavelengths of light, where each wavelength of light propagates at a different speed. The optical fiber may include a core defining a core diameter, where light from the light source passes through the core. The optical fiber may have any suitable core diameter. In some cases, the core diameter of the optical fiber is 10 m or more, e.g., 20 m or more, 30 m or more, 40 m or more, 50 m or more, 60 m or more, including 80 m or more, and is 1,000 m or less, e.g., 500 m or less, 200 m or less, 100 m or less, including 70 m or less. In some embodiments, the core diameter of the optical fiber is in the range of 10 to 1,000 m, e.g., 20 to 500 m, 30 to 200 m, including 40 to 100 km.

In some cases, the systems include a plurality of optical light sources, e.g., a plurality of optical fibers. In some cases, each of the plurality of optical fibers may be reversibly inserted in a different brain region. In some cases, each of the plurality of optical fibers may be implanted in a different brain region. Each of the optical fibers may deliver light pulses having the same or different parameters, e.g., frequency, wavelength, pulse width, intensity, etc. The number of optical fibers used in the present systems may vary, and may be any suitable number. In some cases, the number of optical fibers used to excite and image different regions of the target tissue, e.g., brain, is one or more, e.g., 2 or more, 3 or more, 4 or more, 5 or more, 6 or more, 7 or more, including 10 or more, and is 100 or less, e.g., 80 or less, 60 or less, 40 or less. 20 or less. 15 or less, 10 or less, 8 or less, 7 or less, 6 or less, including 5 or less. In certain embodiments, the number of optical fibers is in the range of 1 to 100, e.g., 2 to 60, 3 to 40, 4 to 20, including, 4 to 10.

In certain cases, a cladding surrounds at least a portion of the core of the optical fiber. For instance, the cladding may surround substantially the entire outer circumferential surface of the optical fiber. In some cases, the cladding is not present on the ends of the optical fiber, such as at the end of the optical fiber that receives light from the light source, and the opposite end of the optical fiber that transmits light to the neurons in the target region of the brain. The cladding may be any suitable type of cladding. In some cases, the cladding has a lower refractive index than the core of the optical fiber. Suitable materials for the cladding include, but are not limited to, plastic, resin, and the like, and combinations thereof.

In some cases, the optical fiber includes an outer coating. The outer coating may be disposed on the surface of the cladding. The coating may surround substantially the entire outer circumferential surface of the optical fiber. In some cases, the coating is not present on the ends of the optical fiber, such as at the end of the optical fiber that receives light from the light source, and the opposite end of the optical fiber that transmits light to the neurons in the target region of the brain. The coating may be a biologically compatible coating. A biologically compatible coating includes coatings that do not significantly react with tissues, fluids, or other substances present in the subject into which the optical fiber is inserted. In some cases, a biologically compatible coating is composed of a material that is inert (i.e., substantially non-reactive) with respect to the surrounding environment in which the optical fiber is used.

The optical fiber end that is implanted or reversibly inserted into the target region of the brain may have any suitable configuration suitable for illuminating a region of the brain with a light stimulus delivered through the optical fiber. In some cases, the optical fiber is removably inserted and/or implanted in the VLO. In some cases, the optical fiber includes an attachment device at or near the distal end of the optical fiber, where the distal end of the optical fiber corresponds to the end inserted into the subject. In some cases, the attachment device is configured to connect to the optical fiber and facilitate attachment of the optical fiber to the subject, such as to the skull of the subject. Any suitable attachment device may be used. In some cases, the attachment device includes a ferrule, e.g., a metal, ceramic or plastic ferrule. The ferrule may have any suitable dimensions for holding and attaching the optical fiber. In some cases, the ferrule has a diameter in the range of 0.5 to 3 mm, e.g., 0.75 to 2.5 mm, or 1 to 2 mm.

In certain embodiments, methods of the present disclosure may be performed using any suitable electronic components to control and/or coordinate the various optical components used to illuminate the regions of the brain. The optical components (e.g., light source, optical fiber, lens, objective, mirror, and the like) may be controlled by a controller, e.g., to coordinate the light source illuminating the regions of the brain with light pulses. The controller may include a driver for the light source that controls one or more parameters associated with the light pulses, such as, but not limited to the frequency, pulse width, duty cycle, wavelength, intensity, etc. of the light pulses. The controllers may be in communication with components of the light source (e.g., collimators, shutters, filter wheels, moveable mirrors, lenses, etc.).

A computational unit (e.g., a computer) may be used in the methods and systems of the present disclosure to control and/or coordinate the light stimulus through the one or more controllers, and to analyze data from fMRI scanning of the regions of the brain. A computational unit may include any suitable components to analyze the measured fMRI images. Thus, the computational unit may include one or more of the following: a processor; a non-transient, computer-readable memory, such as a computer-readable medium; an input device, such as a keyboard, mouse, touchscreen, etc.; an output device, such as a monitor, screen, speaker, etc.; a network interface, such as a wired or wireless network interface; and the like.

The optical light source used to activate the light-activated polypeptide may include light pulses characterized by, e.g., frequency, pulse width, duty cycle, wavelength, intensity, etc. In some cases, the light stimulus includes two or more different sets of light pulses, where each set of light pulses is characterized by different temporal patterns of light pulses. The temporal pattern may be characterized by any suitable parameter, including, but not limited to, frequency, period (i.e., total duration of the light stimulus), pulse width, duty cycle, etc.

The variation in the property of the light pulses of a set may be reflected in a difference in the activity of the illuminated neurons. In some cases, where the neuron is depolarized by activation of the light-activated polypeptide, an increase in the frequency of the light pulses may cause an increase in the frequency of action potential firing in the illuminated neurons. In some embodiments, the frequency of action potential firing in the illuminated neurons scales quantitatively with the increase in the frequency of the light pulses. In some cases, a linear increase in the frequency of the light pulses may induce a linear, or non-linear but monotonic, increase in the frequency of action potential firing in the illuminated neurons. In some instances, stimulation may be manifested as downregulation of neuronal activity, e.g., neuronal hyperpolarization. In some cases, where the neuron is hyperpolarized by activation of the light-activated polypeptide, an increase in the frequency of the light pulses may cause a decrease in the frequency of action potential firing in the illuminated neurons.

In some cases, the light stimulus contains one or more, two or more, three or more, four or more, five or more, six or more, seven of more, eight or more, nine or more, or ten or more sets of light pulses, where the sets of light pulses are characterized by having different parameter values, such as different frequencies of light pulses. Where the sets of light pulses have different frequencies, the duty cycle may be the same, or may be different. In some cases, the sets of light pulses with different frequencies have the same pulse width. In other instances, the sets of light pulses with different frequencies have different pulse widths.

The light pulses of a set may have any suitable frequency. In some cases, the set of light pulses contains a single pulse of light that is sustained throughout the duration of the light stimulus. In some cases, the light pulses of a set have a frequency of 0.1 Hz or more, e.g., 0.5 Hz or more, 1 Hz or more, 5 Hz or more, 10 Hz or more, 20 Hz or more, 30 Hz or more, 40 H or more, including 50 Hz or more, or 60 Hz or more, or 70 Hz or more, or 80 Hz or more, or 90 Hz or more, or 100 Hz or more, and have a frequency of 100,000 Hz or less, e.g., 10,000 Hz or less, 1,000 Hz or less, 500 Hz or less, 400 Hz or less, 300 Hz or less, 200 Hz or less, including 100 Hz or less. In some cases, the light pulses of a set have a frequency in the range of 0.1 to 100,000 Hz, e.g., 1 to 10,000 Hz, 1 to 1,000 Hz, including 5 to 500 Hz, or 10 to 100 Hz. In some embodiments, the light pulse has a frequency ranging from 5 Hz to 40 Hz.

The light pulses of the present system may have any suitable pulse width. In some cases, the pulse width is 0.1 ms or longer, e.g., 0.5 ms or longer, 1 ms or longer, 3 ms or longer, 5 ms or longer, 7.5 ms or longer, 10 ms or longer, including 15 ms or longer, or 20 ms or longer, or 25 ms or longer, or 30 ms or longer, or 35 ms or longer, or 40 ms or longer, or 45 ms or longer, or 50 ms or longer, and is 500 ms or shorter, e.g., 100 ms or shorter, 90 ms or shorter, 80 ms or shorter, 70 ms or shorter, 60 ms or shorter, 50 ms or shorter, 45 ms or shorter, 40 ms or shorter, 35 ms or shorter, 30 ms or shorter, 25 ms or shorter, including 20 ms or shorter. In some embodiments, the pulse width is in the range of 0.1 to 500 ms, e.g., 0.5 to 100 ms, 1 to 80 ms, including 1 to 60 ms, or 1 to 50 ms, or 1 to 30 ms.

The duty cycle of the pulses of the present system may be any suitable duty cycle. In some cases, the duty cycle is 1% or more, e.g., 5% or more, 10% or more, 15% or more, 20% or more including 25% or more, or 30% or more, or 35% or more, or 40% or more, or 45% or more, or 50% or more, and may be 80% or less, e.g., 75% or less, 70% or less, 65% or less, 60% or less, 65% or less, 50% or less, 45% or less, including 40% or less, or 35% or less, or 30% or less. In certain embodiments, the duty cycle is in the range of 1 to 80%, e.g., 5 to 70%, 5 to 60%, including 10 to 50%, or 10 to 40%.

The average power of the light pulse of the present system, measured at the tip of an optical fiber delivering the light pulse to regions of the brain, may be any suitable power. In some cases, the power is 0.1 mW or more, e.g., 0.5 mW or more, 1 mW or more, 1.5 mW or more, including 2 mW or more, or 2.5 mW or more, or 3 mW or more, or 3.5 mW or more, or 4 mW or more, or 4.5 mW or more, or 5 mW or more, and may be 1,000 mW or less, e.g., 500 mW or less, 250 mW or less, 100 mW or less, 50 mW or less, 40 mW or less, 30 mW or less, 20 mW or less, 15 mW or less, including 10 mW or less, or 5 mW or less. In some embodiments, the power is in the range of 0.1 to 1,000 mW, e.g., 0.5 to 100 mW, 0.5 to 50 mW, 1 to 20 mW, including 1 to 10 mW, or 1 to 5 mW.

The wavelength and intensity of the light pulses of the present system may vary and may depend on the activation wavelength of the light-activated polypeptide, optical transparency of the region of the brain, the desired volume of the brain to be illuminated, etc.

The volume of a brain region illuminated by the light pulses may be any suitable volume. In some cases, the illuminated volume is 0.001 mm³ or more, e.g., 0.005 mm³ or more, 0.001 mm³ or more, 0.005 mm³ or more, 0.01 mm³ or more, 0.05 mm³ or more, including 0.1 mm³ or more, and is 100 mm³ or less, e.g., 50 mm³ or less, 20 mm³ or less, 10 mm³ or less, 5 mm³ or less, 1 mm³ or less, including 0.1 mm³ or less. In certain cases, the illuminated volume is in the range of 0.001 to 100 mm³, e.g., 0.005 to 20 mm³, 0.01 to 10 mm³, 0.01 to 5 mm³, including 0.05 to 1 mm³.

Aspects of the present system includes a second light-activated polypeptide expressed in neurons in one or more brain regions of interest. In some cases, the second light-activated polypeptide is administered into the zona incerta (ZI) region of the brain. In some cases, the second light-activated polypeptide is a depolarizing light-activated polypeptide. In some cases, the second light-activated polypeptide is a hyperpolarizing light-activated polypeptide. In some cases, the system of the present disclosure includes stimulation of the ZI region of the brain with an optical light source, for example, when a second light-activated polypeptide is expressed in neurons of the ZI.

The responses to the stimulation by different sets of light pulses may be measured by any suitable brain imaging or neuronal activity measurement system, e.g., fMRI, for the whole brain, and a comparison of the responses in each region may indicate a functional connection between neurons stimulated by the light stimulus to the one or more of thalamocortical projections, thalamic relay neurons, cortical projection neurons, cell bodies in a thalamic submedial nucleus, and cell bodies in the VLO and other regions of the brain, such as on thalamic brain regions downstream from their projection site. In some cases, a quantitative change in the light pulse may cause a change in sign of the fMRI CBV (e.g., a positive or negative CBV response is measured depending on the frequency of the light pulses).

In some cases, the system of the present disclosure includes a fMRI device for measuring a fMRI signal of the whole-brain during stimulation of the one or more of thalamocortical projections, thalamic relay neurons, cortical projection neurons, cell bodies in a thalamic submedial nucleus, and cell bodies in the VLO in the brain. In some cases, fMRI signals are measured in the ipsilateral region, which includes a left hemisphere of the brain comprising a medial prefontal cortex, a lateral prefontal cortex, a motor cortex, a cingulate cortex, a sensory cortex, an insular cortex, a striatum, and a thalamus. In some cases, the system includes a fMRI device for measuring fMRI signals in the contralateral region of the brain, which includes a right hemisphere of the brain comprising a medial prefontal cortex, a lateral prefontal cortex, a motor cortex, a cingulate cortex, a sensory cortex, an insular cortex, a striatum, and a thalamus.

In certain embodiments, an fMRI device may be used to indirectly measure neuronal activity in one or more regions of the brain. For example, fMRI can be used to indirectly measure neuronal activity in different regions of the brain before, during, or after stimulating or illuminating, e.g., with an optical light source, a first region of the brain with a first set of light pulses and a second set of light pulses that have a different temporal pattern, where neurons in the first region may generate action potentials induced by the first set and/or second set of light pulses, or inhibit action potentials following the first set and/or second set of light pulses. In some cases, an increase in neural activity induced by a set of light pulses, e.g., the first set of light pulses, in a region of the brain as provided herein can be associated with a measured fMRI signal. In addition, a decrease in neural activity induced by a set of light pulses, e.g., the second set of light pulses, in the a region of the brain as provided herein can also be associated with a measured fMRI signal. In some cases, a negative measured fMRI signal is associated with a decrease in neuronal activity induced by a set of light pulses in one or more brain regions. In some cases, a positive measured fMRI signal is associated with an increase in neuronal activity induced by a set of light pulses in one or more brain regions. In some cases, a negative measured fMRI signal is associated with a decrease in neuronal activity following stimulation to the one or more of thalamocortical projections, thalamic relay neurons, cortical projection neurons, cell bodies in a thalamic submedial nucleus, and cell bodies of the VLO. In some cases, a positive measured fMRI signal is associated with an increase in neuronal activity following stimulation to the one or more of thalamocortical projections, thalamic relay neurons, cortical projection neurons, cell bodies in a thalamic submedial nucleus, and cell bodies of the VLO.

fMRI may be performed using any suitable method. Suitable methods are described in, e.g., U.S. Pat. No. 8,834,546 and U.S. Patent Pub. No. 2013/0144153A1, which are hereby incorporated by reference in its entirety. Functional magnetic resonance imaging (fMRI) allows the visualization of regions of brain activity with high spatial resolutions (millimeters), in accordance to the tasks being performed by a subject inside a scanner. Functional imaging may include fMRI, and any functional imaging protocols using genetically encoded indicators (e.g., calcium indicators, voltage indicators, etc.). fMRI may be conducted in any suitable static magnetic field (e.g., >1 Tesla) with any suitable accompanying dynamic spatially varying magnetic fields. In some cases, fMRI signals represent CBV in one or more regions of the brain. Suitable fMRI methods and apparatuses are further described in, e.g., Glover. Neurosurg Clin N Am. (2011) 22(2):133-139 and Chow et al. World J Radiol. (2017) 9(1):5-9, the disclosures of which are incorporated herein by reference in their entireties.

The response to stimulation, e.g., as measured by fMRI, may be dependent on the frequency of the light pulse and/or the collection of neurons or brain region that is illuminated. For example, the frequency of the light pulse may determine whether the fMRI signal in one or more brain regions is positive or negative. In some cases, the light pulse is delivered at a frequency that results in a negative measured fMRI signal. In some cases, the light pulse is delivered at a frequency that results in a positive measured fMRI signal. In some instances, stimulating a first brain region with a light pulse results in a negative fMRI signal in one or more downstream brain regions, e.g., brain regions that receive input from the first brain region. In some instances, stimulating a first brain region with a light pulse results in a positive fMRI signal in one or more downstream brain regions.

In some cases, the light pulse has a frequency of 5 Hz or more. In some cases, stimulation of thalamocortical projections with the pulse having a frequency of 5 Hz or more results in a negative measured fMRI signal. In some cases, the negative measured fMRI signal is in the sensory, motor, and cingulate cortex of the ipsilateral region of the brain. In some cases, stimulation of thalamocortical projections with the pulse having a frequency of 5 Hz or more results in the negative measured fMRI in the contralateral region of the brain. In some cases, the negative measured fMRI signal is associated with a decrease in neuronal activity in the contralateral region of the brain. In some instances, stimulation the thalamocortical projections with the light pulse having a frequency of 5 Hz or more inhibits the neuronal activity of the ipsilateral thalamus of the brain.

In some cases, the light pulse has a frequency of 10 Hz or more. In some cases, stimulation of thalamocortical projections with the pulse having a frequency of 10 Hz or more results in a negative measured fMRI signal. In some cases, the negative measured fMRI signal is in the sensory, motor, and cingulate cortex of the ipsilateral region of the brain. In some cases, stimulation of thalamocortical projections with the pulse having a frequency of 10 Hz or more results in a negative measured fMRI signal. In some cases, the negative measured fMRI signal is in the sensory, motor, and cingulate cortex of the ipsilateral region of the brain. In some cases, the negative measured fMRI signal is associated with a decrease in neuronal activity in the sensory, motor, and cingulate cortex of the ipsilateral region of the brain. In some cases, stimulation of the thalamocortical projection with the light pulse having a frequency of 10 Hz or more results in the negative measured fMRI signal in the contralateral region of the brain. In some cases, the negative measured fMRI signal is associated with a decrease in neuronal activity in the contralateral region of the brain. In some cases, stimulating the thalamocortical projections with the light pulse having a frequency of 10 Hz or more results in the negative measured fMRI signal in the cortex, contralateral striatum, and contralateral thalamus of the brain.

In some cases, the light pulse has a frequency ranging from 5 Hz to 20 Hz. In some cases, stimulation of the thalamocortical projections at the light pulse having a frequency ranging from 5 Hz to 20 Hz results in a negative measured fMRI signal in the contralateral region of the brain. In some cases, stimulation of the thalamocortical projections at the light pulse having a frequency ranging from 5 Hz to 20 Hz inhibits neuronal activity in the contralateral region of the brain. In some cases, the contralateral region comprises the prefrontal cortex of the brain. In some cases, the negative measured fMRI signal is associated with a decrease in neuronal activity in the contralateral region of the brain. In some cases, stimulation of thalamocortical projections with the light pulse having a frequency ranging from 5 Hz or more, 10 Hz or more, 15 Hz or more, or 20 Hz inhibits the neuronal activity of the contralateral region of the brain.

In some cases, the light pulse has a frequency ranging from 20 Hz to 40 Hz. In some cases, stimulation of thalamocortical projections with the pulse having a frequency ranging from 20 Hz to 40 Hz results in a positive measured fMRI signal. In some cases, the positive measured fMRI signal is associated with an increase in neuronal activity in the ipsilateral region of the brain. In some cases, the positive measured fMRI signal is associated with an increase in neuronal activity in the ipsilateral thalamus of the brain. In some cases, stimulation of thalamocortical projections with the light pulse having a frequency ranging from 20 Hz or more, 25 Hz or more, 30 Hz or more, 35 Hz or more, or 40 Hz or more results in the positive measured fMRI signal in the ipsilateral region of the brain associated with an increase in neuronal activity in the ipsilateral region of the brain. In some instances, stimulating the thalamocortical projections with the light pulse having a frequency ranging from 20-40 Hz activates the neuronal activity of the ipsilateral thalamus of the brain. In some instances, stimulation thalamocortical projections with the light pulse having a frequency ranging from 25 Hz or more results in a negative measured fMRI signal in the contralateral region of the brain.

In some cases, the light pulse has a frequency of 40 Hz or more. In some cases, stimulation of thalamocortical projections with the pulse having a frequency of 40 Hz or more results in a positive measured fMRI signal. In some cases, stimulation of thalamocortical projections with the light pulse with a frequency of 40 Hz or more results in a positive fMRI signal in the ipsilateral region of the brain. In some cases, the positive measured fMRI signal is associated with an increase in neuronal activity in the ipsilateral region of the brain. In some cases, the positive measured fMRI signal is associated with an increase in neuronal activity in the ipsilateral thalamus of the brain. In some instances, stimulation of thalamocortical projections with the light pulse with a frequency of 40 Hz or more results in a positive fMRI signal in the ipsilateral thalamus, ipsilateral striatum, and ipsilateral cortex of the brain. In some cases, the light pulse has a frequency ranging from 5 Hz to 40 Hz. In some cases, stimulation of the cell bodies in the VLO with the light pulse having a frequency ranging from 5 Hz to 40 Hz results in the positive measured fMRI signal in the ipsilateral region of the brain. In some cases, stimulation of the cell bodies in the VLO with the light pulse having a frequency ranging from 5 Hz or more, 10 Hz or more, 15 Hz or more, 20 Hz or more, 25 Hz or more, 30 Hz or more, 35 Hz or more, or 40 Hz or more results in the positive measured fMRI signal in the ipsilateral region of the brain. In some cases, the positive measured fMRI signal is associated with an increase in neuronal activity in the ipsilateral thalamus of the brain.

In some cases, the light pulse has a frequency ranging from 5 Hz to 40 Hz. In some cases, stimulation of the cell bodies of the thalamic submedial nucleus results in a positive measured fMRI signal in the ipsilateral thalamus of the brain. In some cases, stimulation of the cell bodies of the thalamic submedial nucleus with the light pulse having a frequency ranging from 5 Hz or more, 10 Hz or more, 15 Hz or more, 20 Hz or more, 25 Hz or more, 30 Hz or more, 35 Hz or more, or 40 Hz or more results in the positive measured fMRI signal in the ipsilateral region of the brain. In some cases, the positive measured fMRI signal is associated with an increase in neuronal activity in the ipsilateral thalamus of the brain.

In some cases, the light pulse has a frequency ranging from 5 Hz to 10 Hz. In some instances, stimulation of thalamocortical projections with the light pulse having a frequency ranging from 5 Hz to 10 Hz decreases brain activity in the ipsilateral region of the brain. In some cases, stimulation with the light pulse having a frequency ranging from 5 Hz to 10 Hz inhibits the neuronal activity of the ipsilateral thalamus of the brain.

Aspects of the present system include an electrophysiological recording device to record and detect firing rates of neurons in one or more brain regions associated with a measured fMRI signal. Electrophysiology may include single electrode, multi electrode, and/or field potential recordings. In some cases, the one or more brain regions comprises the ipsilateral VLO of the brain. In some cases, a positive measured fMRI signal is associated with an increased firing rate of neurons recorded in the ipsilateral VLO. In some cases, a negative measured fMRI signal is associated with a decreased firing rate of neurons recorded in the ipsilateral VLO. In some cases, stimulation of at the light pulse having a frequency of 10 Hz or more results in a decrease in firing rate of neurons in the ipsilateral motor cortex. In some cases, stimulation of at the light pulse having a frequency of 40 Hz or more results in an increase in firing rate of neurons in the ipsilateral motor cortex.

In some cases, the one or more brain regions comprises the contralateral VLO of the brain. In some cases, a negative measured fMRI signal is associated with a decreased firing rate of neurons in the contralateral VLO. In some cases, stimulation of the contralateral VLO associated with the negative measured fMRI signal is associated with a decreased firing rate of neurons in the contralateral VLO. In some cases, stimulation of the contralateral VLO at the light pulse having a frequency of 10 Hz or more results in a decreased firing rate of neurons in the contralateral VLO. In some cases, stimulation of at the light pulse having a frequency of 40 Hz or more results in an increase in firing rate of neurons in the contralateral VLO.

Electrophysiological recordings may be performed using any suitable protocol and device. In some cases, the electrophysiological recordings include intracellular recordings. In some instances, performing the recordings include inserting a microelectrode into the interior of a neuron. In some instances, performing the recordings include placing a microelectrode on a surface of a cell membrane of a neuron.

In some instances, performing the recordings utilizes methods and apparatuses for performing patch clamp electrophysiology and any variations including, e.g., whole-cell, inside-out, outside-out, perforated, loose patch clamp methods. In some instances, the recordings are performed using the voltage clamp method. In some instances, the recordings are performed using the current clamp method. In some cases, the recordings include extracellular recordings which may detect changes in ion concentrations in the extracellular fluid or in a group of neurons. Electrophysiology may include single electrode, multi electrode, and/or field potential recordings. In some instances, an electrode is a glass micropipette. In some instances, the recordings are performed with a plurality of electrodes, e.g., a microelectrode array. Exemplary methods and apparatuses for performing electrophysiological recordings are described in, e.g., U.S. Pat. Pub. Nos. 2013/0225963; 2017/0138926; and 2005/0231186, the disclosures of which are incorporated herein by reference in their entireties. Exemplary electrode technologies for neural recordings are described in Hong et al. Nat Rev Neurosci (2019) 20(6):330-345, the disclosure of which is incorporated herein by reference in its entirety. Light-induced modulation of neural activity may include any suitable optogenetic method, as described herein. In some cases, the electrophysiological recordings include single-unit recordings.

Aspects of the present disclosure further include a system of modulating pain in an individual. In some cases, the system includes i) an optical light source configured to stimulate one or more of thalamocortical projections, thalamic relay neurons, cortical projection neurons, cell bodies in a thalamic submedial nucleus, and cell bodies in the VLO in the brain of the individual with one or more light pulses, wherein neuronal cell bodies in one or more of the VLO and a thalamus of an individual expresses a light-activated polypeptide, and wherein said stimulation modulates pain in an individual.

In some cases, stimulation of one or more of thalamocortical projections, thalamic relay neurons, cortical projection neurons, cell bodies in a thalamic submedial nucleus, and cell bodies in the VLO in the brain with a first set of light pulses inhibits the neuronal activity in response to noxious stimuli.

In some cases, stimulation of one or more of thalamocortical projections, thalamic relay neurons, cortical projection neurons, cell bodies in a thalamic submedial nucleus, and cell bodies in the VLO in the brain with a first set of light pulses inhibits the neuronal activity associated with aversive or painful sensations in the orbitofrontal cortex of the brain.

In some cases, stimulation of one or more of thalamocortical projections, thalamic relay neurons, cortical projection neurons, cell bodies in a thalamic submedial nucleus, and cell bodies in the VLO in the brain with a second set of light pulses activates the neuronal activity associated with aversive or painful sensations in the orbitofrontal cortex of the brain.

In some cases, stimulation of one or more of thalamocortical projections, thalamic relay neurons, cortical projection neurons, cell bodies in a thalamic submedial nucleus, and cell bodies in the VLO in the brain with a second set of light pulses activates the neuronal activity associated with aversive or painful sensations in the orbitofrontal cortex of the brain.

### Light-Activated Polypeptides Used in the Method and Systems

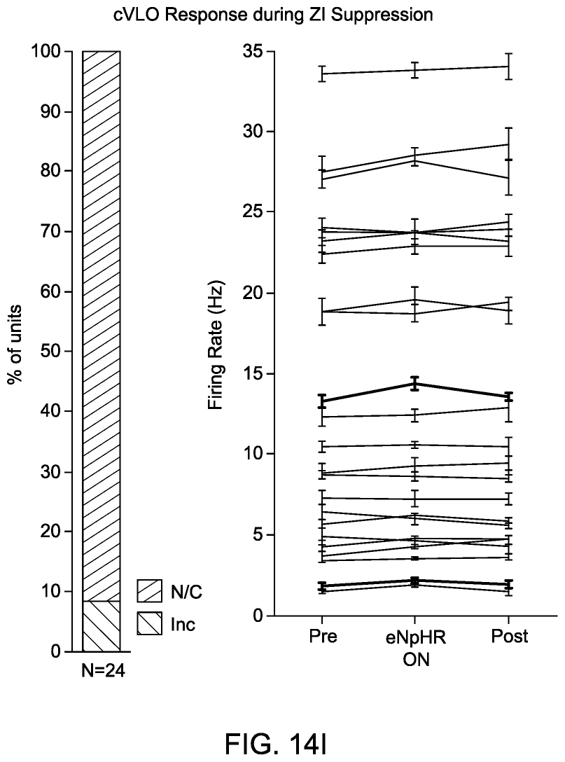

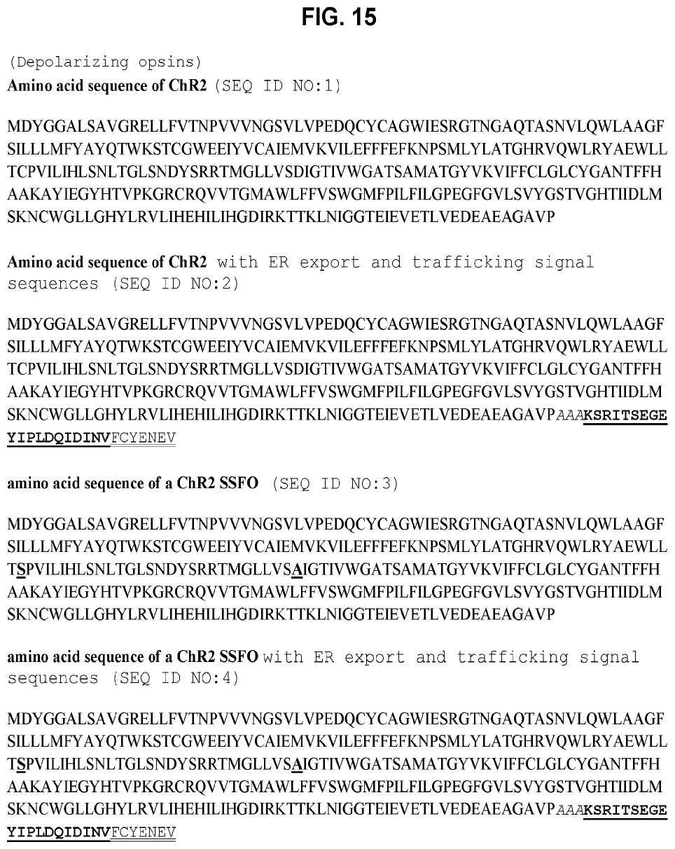

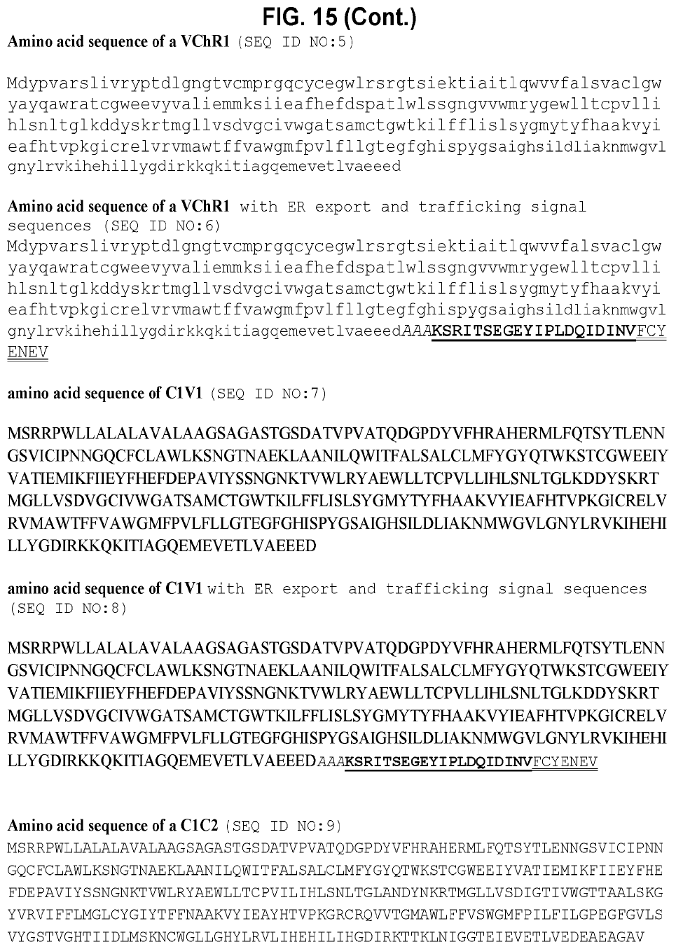

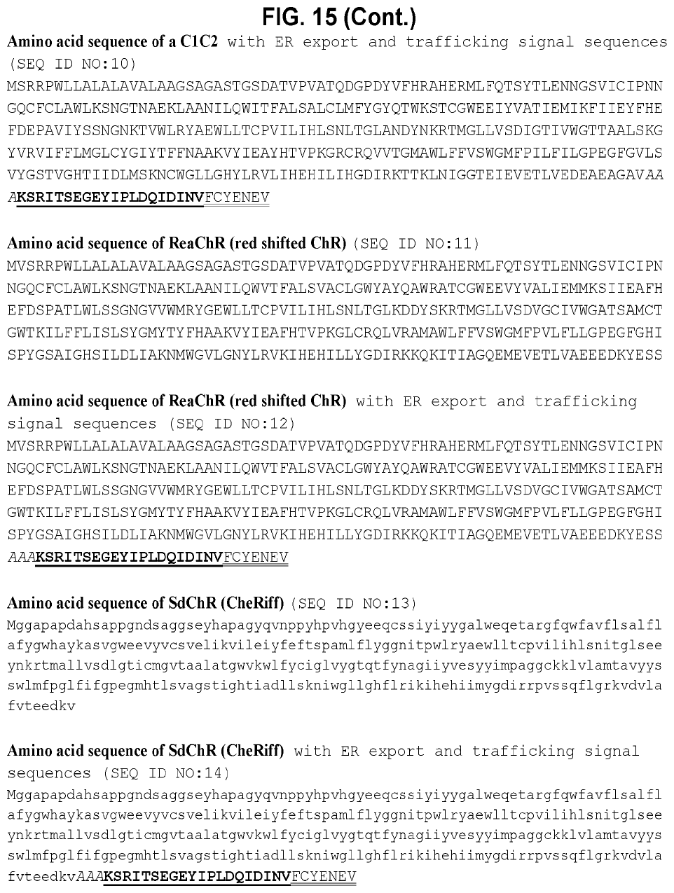

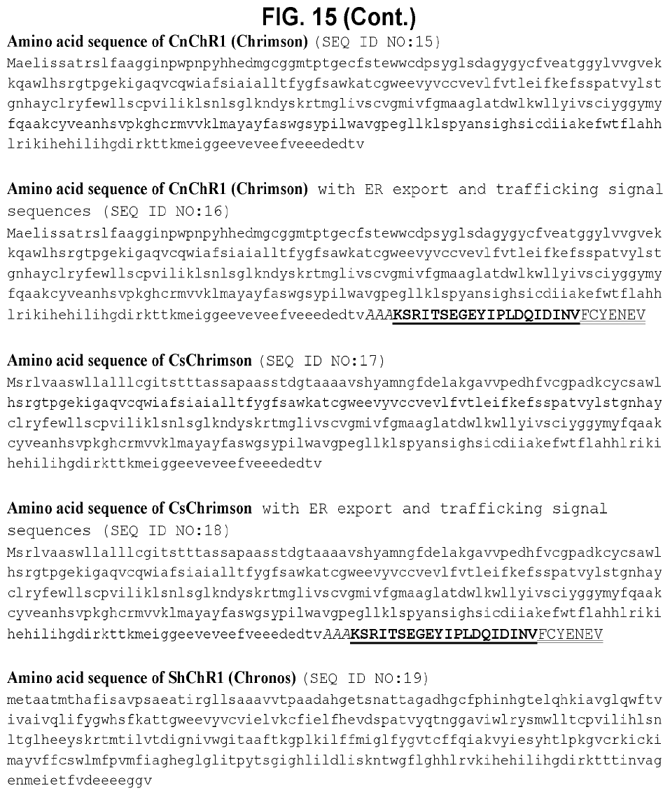

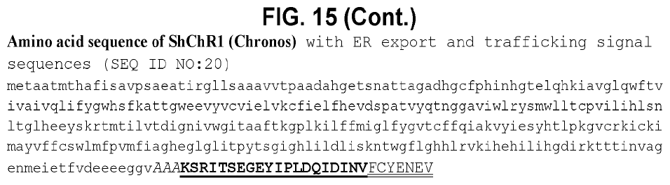

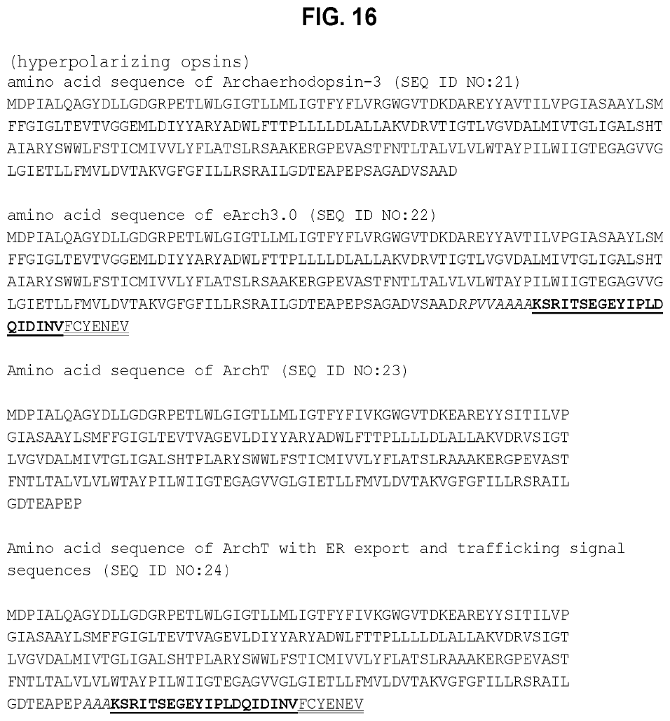

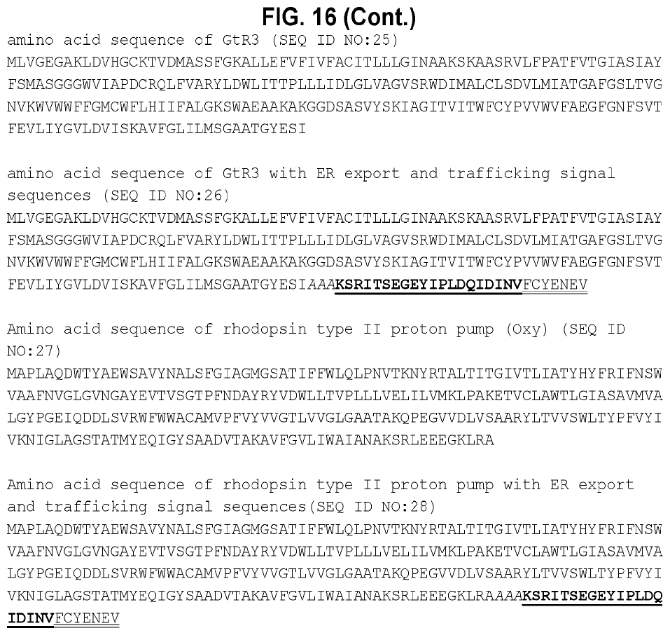

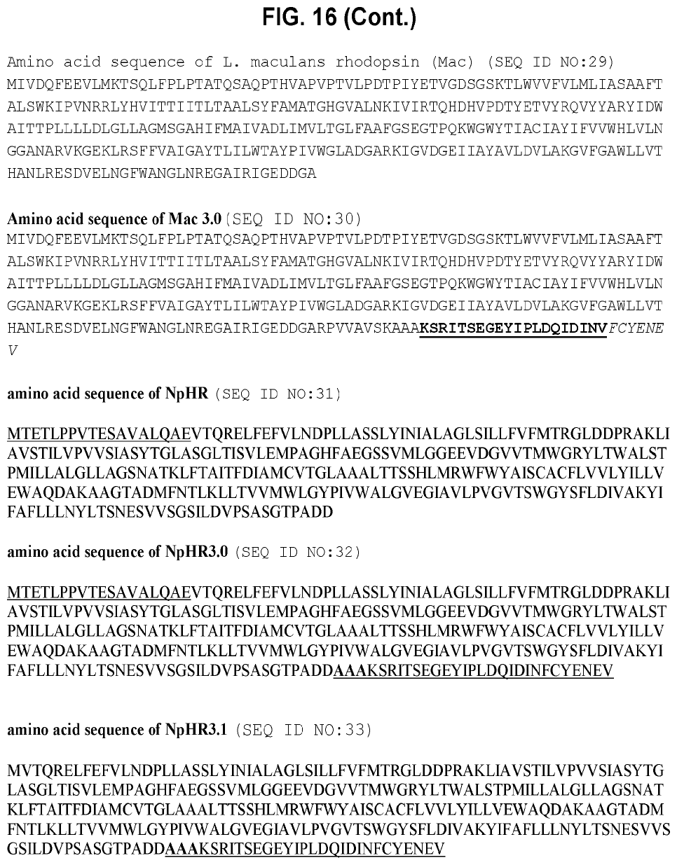

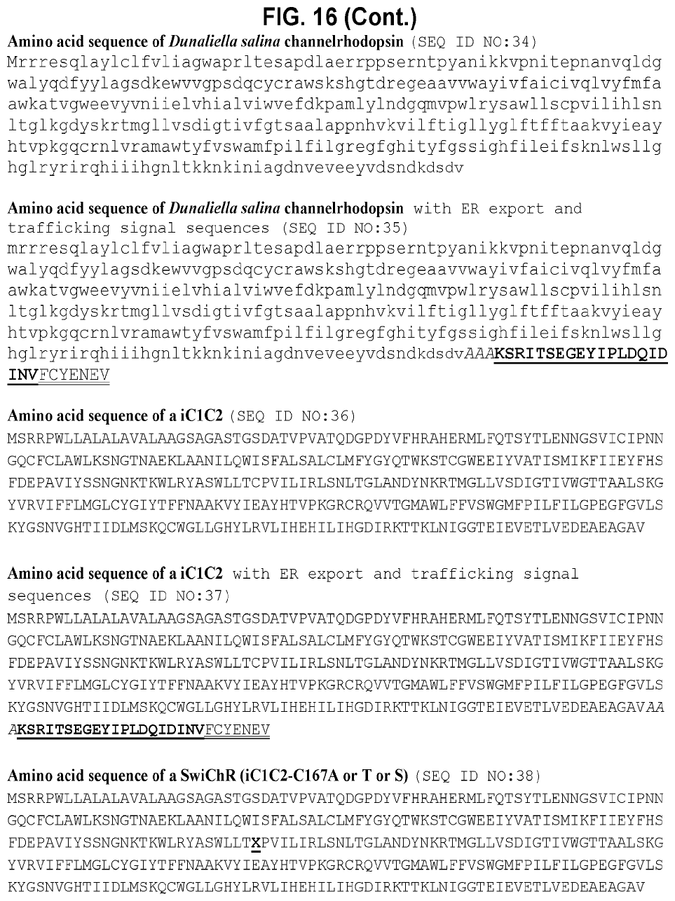

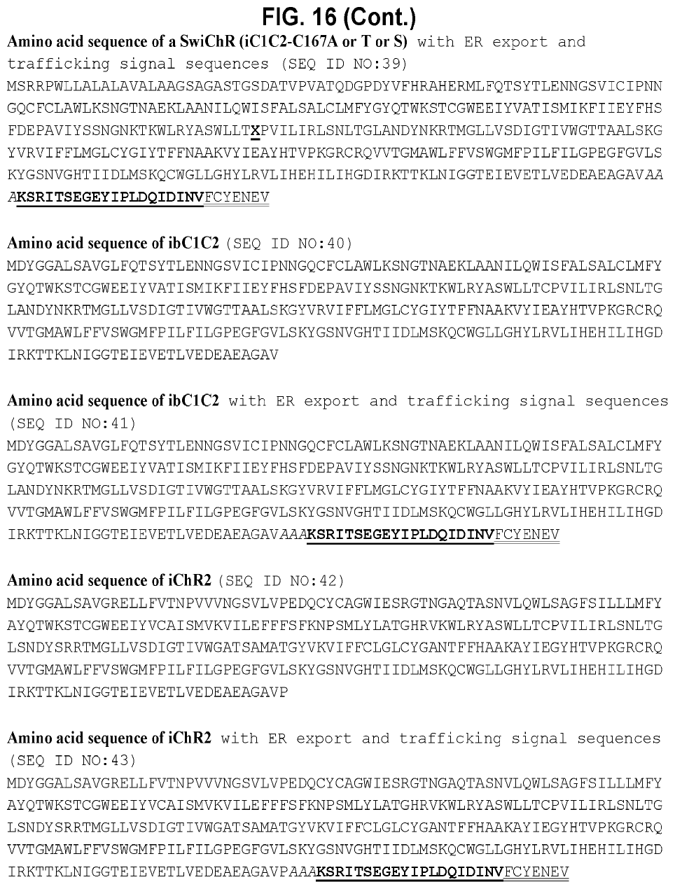

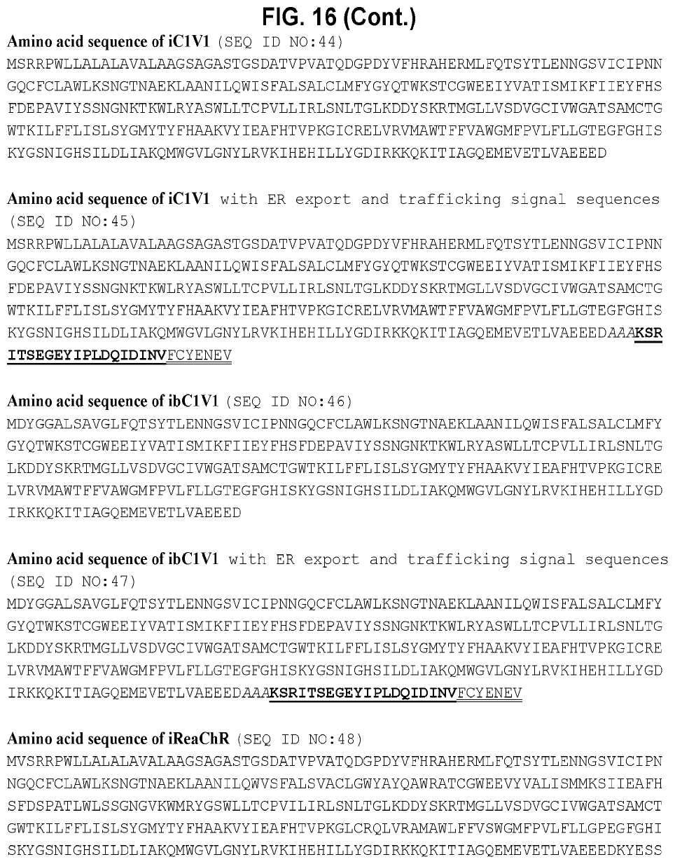

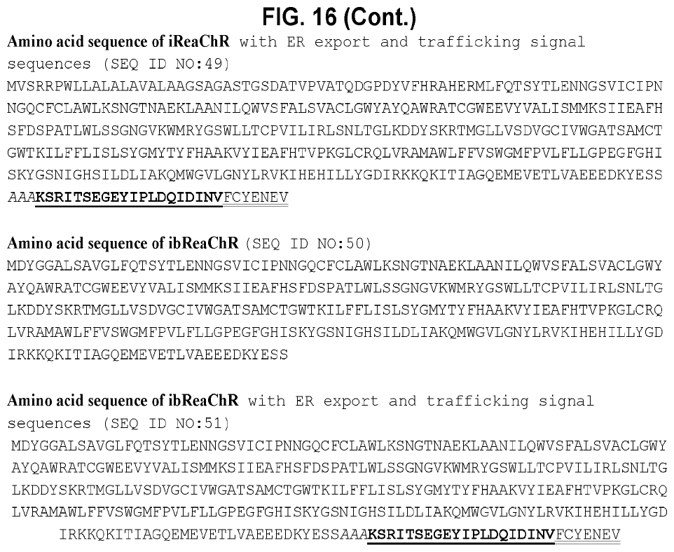

As summarized above, aspects of the present methods and systems include various brain regions containing neurons with, e.g., expressing, a light-activated polypeptide. The light-activated polypeptide may be a light-activated ion channel or a light-activated ion pump. The light-activated ion channel polypeptides are adapted to allow one or more ions to pass through the plasma membrane of a neuron when the polypeptide is illuminated with light of an activating wavelength. Light-activated proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-activated polypeptide depolarizes the neuron when activated by light of an activating wavelength. Suitable depolarizing light-activated polypeptides, without limitation, are shown in FIG. 15. In some embodiments, the light-activated polypeptide hyperpolarizes the neuron when activated by light of an activating wavelength. Suitable hyperpolarizing light-activated polypeptides, without limitation, are shown in FIG. 16.

In some embodiments, the light-activated polypeptides are activated by blue light. In some embodiments, the light-activated polypeptides are activated by green light. In some embodiments, the light-activated polypeptides are activated by yellow light. In some embodiments, the light-activated polypeptides are activated by orange light. In some embodiments, the light-activated polypeptides are activated by red light.

In some embodiments, the light-activated polypeptide expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-activated protein transport to the plasma membranes of mammalian cells can be fused to the N-terminus, the C-terminus, or to both the N- and C-terminal ends of the light-activated polypeptide. In some cases, the one or more amino acid sequence motifs which enhance light-activated polypeptide transport to the plasma membranes of mammalian cells is fused internally within a light-activated polypeptide. Optionally, the light-activated polypeptide and the one or more amino acid sequence motifs may be separated by a linker.

In some embodiments, the light-activated polypeptide can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)).

A trafficking sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

ER export sequences that are suitable for use with a light-activated polypeptide include, e.g., VXXSL (where X is any amino acid; SEQ ID NO:52) (e.g., VKESL (SEQ ID NO:53); VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

Signal sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such as one of the following: 1) the signal peptide of hChR2 (e.g., MDYGGALSAVGRELLFVTNPVVVNGS (SEQ ID NO:59)); 2) the β2 subunit signal peptide of the neuronal nicotinic acetylcholine receptor (e.g., MAGHSNSMALFSFSLLWLCSGVLGTEF (SEQ ID NO:60)); 3) a nicotinic acetylcholine receptor signal sequence (e.g., MGLRALMLWLLAAAGLVRESLQG (SEQ ID NO:64)); and 4) a nicotinic acetylcholine receptor signal sequence (e.g., MRGTPLLLVVSLFSLLQD (SEQ ID NO:61)).

A signal sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

In some embodiments, the signal peptide sequence in the protein can be deleted or substituted with a signal peptide sequence from a different protein.

Examples of light-activated polypeptides are described in, e.g., PCT App. No. PCT/US2011/028893, which is hereby incorporated by reference in its entirety. Representative light-activated polypeptides that find use in the present disclosure are further described below.

### Depolarizing Light-Activated Polypeptides

### ChR

In some aspects, a depolarizing light-activated polypeptide is derived from Chlamydomonas reinhardtii, wherein the polypeptide is capable of transporting cations across a cell membrane when the cell is illuminated with light. In another embodiment, the light-activated polypeptide comprises an amino acid sequence at least 75%, 85%, 90%, 91%, 92%, 93%, 94%, 95%, 96%, 97%, 98%, 99%, or 100% identical to the sequence shown in SEQ ID NO:1. The light used to activate the light-activated cation channel protein derived from Chlamydomonas reinhardtii can have a wavelength between about 460 and about 495 nm or can have a wavelength of about 480 nm. Additionally, light pulses having a temporal frequency of about 100 Hz can be used to activate the light-activated protein. In some embodiments, activation of the light-activated cation channel derived from Chlamydomonas reinhardtii with light pulses having a temporal frequency of about 100 Hz can cause depolarization of the neurons expressing the light-activated cation channel. The light-activated cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-activated cation channel protein to regulate the polarization state of the plasma membrane of the cell. Additionally, the light-activated cation channel protein can comprise one or more conservative amino acid substitutions and/or one or more non-conservative amino acid substitutions. The light-activated proton pump protein containing substitutions, deletions, and/or insertions introduced into the native amino acid sequence suitably retains the ability to transport cations across a cell membrane.

In some embodiments, the light-activated cation channel includes a T159C substitution of the amino acid sequence set forth in SEQ ID NO:1. In some embodiments, the light-activated cation channel includes a L132C substitution of the amino acid sequence set forth in SEQ ID NO:1. In some embodiments, the light-activated cation channel includes an E123T substitution of the amino acid sequence set forth in SEQ ID NO:1. In some embodiments, the light-activated cation channel includes an E123A substitution of the amino acid sequence set forth in SEQ ID NO:1. In some embodiments, the light-activated cation channel includes a T159C substitution and an E123T substitution of the amino acid sequence set forth in SEQ ID NO:1. In some embodiments, the light-activated cation channel includes a T159C substitution and an E123A substitution of the amino acid sequence set forth in SEQ ID NO:1. In some embodiments, the light-activated cation channel includes a T159C substitution, an L132C substitution, and an E123T substitution of the amino acid sequence set forth in SEQ ID NO:1. In some embodiments, the light-activated cation channel includes a T159C substitution, an L132C substitution, and an E123A substitution of the amino acid sequence set forth in SEQ ID NO:1. In some embodiments, the light-activated cation channel includes an L132C substitution and an E123T substitution of the amino acid sequence set forth in SEQ ID NO:1. In some embodiments, the light-activated cation channel includes an L132C substitution and an E123A substitution of the amino acid sequence set forth in SEQ ID NO:1.

In some embodiments, a ChR2 protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the ChR2 protein comprises an N-terminal signal peptide and a C-terminal ER export signal. In some embodiments, the ChR2 protein comprises an N-terminal signal peptide and a C-terminal trafficking signal. In some embodiments, the ChR2 protein comprises an N-terminal signal peptide, a C-terminal ER export signal, and a C-terminal trafficking signal. In some embodiments, the ChR2 protein comprises a C-terminal ER export signal and a C-terminal trafficking signal. In some embodiments, the C-terminal ER export signal and the C-terminal trafficking signal are linked by a linker. The linker can comprise any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the ChR2 protein can have an amino acid sequence that is at least 75%, 85%, 90%, 91%, 92%, 93%, 94%, 95%, 96%, 97%, 98%, 99%, or 100% identical to the sequence shown in SEQ ID NO:2.

In other embodiments, the light-activated polypeptide is a step function opsin (SFO) protein or a stabilized step function opsin (SSFO) protein that can have specific amino acid substitutions at key positions in the retinal binding pocket of the protein. In some embodiments, the SFO protein can have a mutation at amino acid residue C128 of SEQ ID NO:1. In other embodiments, the SFO protein has a C128A mutation in SEQ ID NO:1. In other embodiments, the SFO protein has a C128S mutation in SEQ ID NO:1. In another embodiment, the SFO protein has a C128T mutation in SEQ ID NO:1.

In some embodiments, the SSFO protein can have a mutation at amino acid residue D156 of SEQ ID NO:1. In other embodiments, the SSFO protein can have a mutation at both amino acid residues C128 and D156 of SEQ ID NO:1. In one embodiment, the SSFO protein has an C128S and a D156A mutation in SEQ ID NO:1.

In another embodiment, the SSFO protein can comprise an amino acid sequence at least 75%, 85%, 90%, 91%, 92%, 93%, 94%, 95%, 96%, 97%, 98%, 99%, or 100% identical to the sequence shown in SEQ ID NO:1; and includes an alanine, serine, or threonine at amino acid 128; and includes a alanine at amino acid 156. In another embodiment, the SSFO protein can comprise a C128T mutation in SEQ ID NO:1. In some embodiments, the SSFO protein includes C128T and D156A mutations in SEQ ID NO:1.

In some embodiments the SFO or SSFO proteins provided herein can be capable of mediating a depolarizing current in the cell when the cell is illuminated with blue light. In other embodiments, the light can have a wavelength of about 445 nm. Additionally, in some embodiments the light can be delivered as a single pulse of light or as spaced pulses of light due to the prolonged stability of SFO and SSFO photocurrents. In some embodiments, activation of the SFO or SSFO protein with single pulses or spaced pulses of light can cause depolarization of a neuron expressing the SFO or SSFO protein. In some embodiments, each of the disclosed step function opsin and stabilized step function opsin proteins can have specific properties and characteristics for use in depolarizing the membrane of a neuronal cell in response to light.

Further disclosure related to SFO or SSFO proteins can be found in International Patent Application Publication No. WO 2010/056970, the disclosure of which is hereby incorporated by reference in its entirety.

In some cases, the ChR2-based SFO or SSFO comprises a membrane trafficking signal and/or an ER export signal. In some embodiments, the trafficking signal is derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the SSFO protein comprises an amino acid sequence that is at least 75%, 85%, 90%, 91%, 92%, 93%, 94%, 95%, 96%, 97%, 98%, 99%, or 100% identical to the sequence shown in SEQ ID NO:4.

### Volvox Carteri Light-Activated Polypeptide

In some embodiments, a suitable light-activated polypeptide is a cation channel derived from Volvox carteri (VChR1) and is activated by illumination with light of a wavelength of from about 500 nm to about 600 nm, e.g., from about 525 nm to about 550 nm, e.g., 545 nm. In some embodiments, the light-activated ion channel protein comprises an amino acid sequence at least 75%, 85%, 90%, 91%, 92%, 93%, 94%, 95%, 96%, 97%, 98%, 99%, or 100% identical to the sequence shown in SEQ ID NO:5. The light-activated ion channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-activated ion channel protein to regulate the polarization state of the plasma membrane of the cell. Additionally, the light-activated ion channel protein can comprise one or more conservative amino acid substitutions and/or one or more non-conservative amino acid substitutions. The light-activated ion channel protein containing substitutions, deletions, and/or insertions introduced into the native amino acid sequence suitably retains the ability to transport ions across the plasma membrane of a neuronal cell in response to light.

In some cases, a VChR1 light-activated cation channel protein comprises a core amino acid sequence at least 75%, 85%, 90%, 91%, 92%, 93%, 94%, 95%, 96%, 97%, 98%, 99%, or 100% identical to the sequence shown in SEQ ID NO:5 and at least one (such as one, two, three, or more) amino acid sequence motifs which enhance transport to the plasma membranes of mammalian cells selected from the group consisting of a signal peptide, an ER export signal, and a membrane trafficking signal. In some embodiments, the light-activated proton ion channel comprises an N-terminal signal peptide and a C-terminal ER export signal. In some embodiments, the light-activated ion channel protein comprises an N-terminal signal peptide and a C-terminal trafficking signal. In some embodiments, the light-activated ion channel protein comprises an N-terminal signal peptide, a C-terminal ER Export signal, and a C-terminal trafficking signal. In some embodiments, the light-activated ion channel protein comprises a C-terminal ER Export signal and a C-terminal trafficking signal. In some embodiments, the C-terminal ER Export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER Export signal is more C-terminally located than the trafficking signal. In some embodiments the trafficking signal is more C-terminally located than the ER Export signal.

In some embodiments, the trafficking signal is derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the VChR1 protein comprises an amino acid sequence that is at least 75%, 85%, 90%, 91%, 92%, 93%, 94%, 95%, 96%, 97%, 98%, 99%, or 100% identical to the sequence shown in SEQ ID NO:6.

### Step Function Opsins and Stabilized Step Function Opsins Based on VChR1

In other embodiments, the light-activated polypeptide is a SFO or an SSFO based on VChR1. In some embodiments, the SFO protein can have a mutation at amino acid residue C123 of SEQ ID NO:5. In other embodiments, the SFO protein has a C123A mutation in SEQ ID NO:5. In other embodiments, the SFO protein has a C123S mutation in SEQ ID NO:5. In another embodiment, the SFO protein has a C123T mutation in SEQ ID NO:5.

In some embodiments, the SFO protein can have a mutation at amino acid residue D151 of SEQ ID NO:5. In other embodiments, the SFO protein can have a mutation at both amino acid residues C123 and D151 of SEQ ID NO:5. In one embodiment, the SFO protein has an C123S and a D151A mutation in SEQ ID NO:5.

In some embodiments an SFO or SSFO protein is capable of mediating a depolarizing current in the cell when the cell is illuminated with blue light. In some embodiments, the light has a wavelength of about 560 nm. Additionally, in some embodiments the light is delivered as a single pulse of light or as spaced pulses of light due to the prolonged stability of SFO and SSFO photocurrents. In some embodiments, activation of the SFO or SSFO protein with single pulses or spaced pulses of light can cause depolarization of a neuron expressing the SFO or SSFO protein. In some embodiments, each of the disclosed step function opsin and stabilized step function opsin proteins can have specific properties and characteristics for use in depolarizing the membrane of a neuronal cell in response to light.

In some cases, the VChR1-based SFO or SSFO comprises a membrane trafficking signal and/or an ER export signal. In some embodiments, the trafficking signal can be derived from the amino acid sequence of the human inward rectifier potassium channel Kir2.1. Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

### C1V1 Chimeric Cation Channels

In other embodiments, the light-activated cation channel protein is a C1V1 chimeric protein derived from the VChR1 protein of Volvox carteri and the ChR1 protein from Chlamydomonas reinhardti, wherein the protein comprises the amino acid sequence of VChR1 having at least the first and second transmembrane helices replaced by the first and second transmembrane helices of ChR1; is responsive to light; and is capable of mediating a depolarizing current in the cell when the cell is illuminated with light. In some embodiments, the C1V1 protein further comprises a replacement within the intracellular loop domain located between the second and third transmembrane helices of the chimeric light responsive protein, wherein at least a portion of the intracellular loop domain is replaced by the corresponding portion from ChR1. In another embodiment, the portion of the intracellular loop domain of the C1V1 chimeric protein can be replaced with the corresponding portion from ChR1 extending to amino acid residue A145 of the ChR1. In other embodiments, the C1V1 chimeric protein further comprises a replacement within the third transmembrane helix of the chimeric light responsive protein, wherein at least a portion of the third transmembrane helix is replaced by the corresponding sequence of ChR1. In yet another embodiment, the portion of the intracellular loop domain of the C1V1 chimeric protein can be replaced with the corresponding portion from ChR1 extending to amino acid residue W163 of the ChR1. In other embodiments, the C1V1 chimeric protein comprises an amino acid sequence at least 75%, 85%, 90%, 91%, 92%, 93%, 94%, 95%, 96%, 97%, 98%, 99%, or 100% identical to the sequence shown in SEQ ID NO:7.

In some embodiments, the C1V1 protein mediates a depolarizing current in the cell when the cell is illuminated with green light. In some embodiments, the light has a wavelength of between about 540 nm to about 560 nm. In some embodiments, the light can have a wavelength of about 542 nm. In some embodiments, the C1V1 chimeric protein is not capable of mediating a depolarizing current in the cell when the cell is illuminated with violet light. In some embodiments, the chimeric protein is not capable of mediating a depolarizing current in the cell when the cell is illuminated with light having a wavelength of about 405 nm. Additionally, in some embodiments, light pulses having a temporal frequency of about 100 Hz can be used to activate the C1V1 protein.

In some cases, the C1V1 polypeptide comprises a membrane trafficking signal and/or an ER export signal. In some embodiments, the trafficking signal is derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the C1V1 protein comprises an amino acid sequence that is at least 75%, 85%, 90%, 91%, 92%, 93%, 94%, 95%, 96%, 97%, 98%, 99%, or 100% identical to the sequence shown in SEQ ID NO:8.

### C1V1 Variants

In some aspects, a suitable light-activated polypeptide comprises substituted or mutated amino acid sequences, wherein the mutant polypeptide retains the characteristic light-activatable nature of the precursor C1V1 chimeric polypeptide but may also possess altered properties in some specific aspects. For example, the mutant light-activated C1V1 chimeric proteins described herein can exhibit an increased level of expression both within an animal cell or on the animal cell plasma membrane; an altered responsiveness when exposed to different wavelengths of light, particularly red light; and/or a combination of traits whereby the chimeric C1V1 polypeptide possess the properties of low desensitization, fast deactivation, low violet-light activation for minimal cross-activation with other light-activated cation channels, and/or strong expression in animal cells.

Accordingly, suitable light-activated proteins include C1V1 chimeric light-activated proteins that can have specific amino acid substitutions at key positions throughout the retinal binding pocket of the VChR1 portion of the chimeric polypeptide. In some embodiments, the C1V1 protein comprises an amino acid substitution at amino acid residue E122 of SEQ ID NO:7. In some embodiments, the C1V1 protein comprises a substitution at amino acid residue E162 of SEQ ID NO:7. In other embodiments, the C1V1 protein comprises a substitution at both amino acid residues E162 and E122 of SEQ ID NO:7.

In some aspects, the C1V1-E122 mutant chimeric protein is capable of mediating a depolarizing current in the cell when the cell is illuminated with light. In some embodiments the light is green light. In other embodiments, the light has a wavelength of between about 540 nm to about 560 nm. In some embodiments, the light has a wavelength of about 546 nm. In other embodiments, the C1V1-E122 mutant chimeric protein mediates a depolarizing current in the cell when the cell is illuminated with red light. In some embodiments, the red light has a wavelength of about 630 nm. In some embodiments, the C1V1-E122 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. Additionally, in some embodiments, light pulses having a temporal frequency of about 100 Hz can be used to activate the C1V1-E122 mutant chimeric protein. In some embodiments, activation of the C1V1-E122 mutant chimeric protein with light pulses having a frequency of 100 Hz can cause depolarization of the neurons expressing the C1V1-E122 mutant chimeric protein.

In other aspects, the C1V1-E162 mutant chimeric protein is capable of mediating a depolarizing current in the cell when the cell is illuminated with light. In some embodiments the light can be green light. In other embodiments, the light can have a wavelength of between about 535 nm to about 540 nm. In some embodiments, the light can have a wavelength of about 542 nm. In other embodiments, the light can have a wavelength of about 530 nm. In some embodiments, the C1V1-E162 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. Additionally, in some embodiments, light pulses having a temporal frequency of about 100 Hz can be used to activate the C1V1-E162 mutant chimeric protein. In some embodiments, activation of the C1V1-E162 mutant chimeric protein with light pulses having a frequency of 100 Hz can cause depolarization-induced synaptic depletion of the neurons expressing the C1V1-E162 mutant chimeric protein.

In yet other aspects, the C1V1-E122/E162 mutant chimeric protein is capable of mediating a depolarizing current in the cell when the cell is illuminated with light. In some embodiments the light can be green light. In other embodiments, the light can have a wavelength of between about 540 nm to about 560 nm. In some embodiments, the light can have a wavelength of about 546 nm. In some embodiments, the C1V1-E122/E162 mutant chimeric protein does not mediate a depolarizing current in the cell when the cell is illuminated with violet light. In some embodiments, the chimeric protein does not mediate a depolarizing current in the cell when the cell is illuminated with light having a wavelength of about 405 nm. In some embodiments, the C1V1-E122/E162 mutant chimeric protein can exhibit less activation when exposed to violet light relative to C1V1 chimeric proteins lacking mutations at E122/E162 or relative to other light-activated cation channel proteins. Additionally, in some embodiments, light pulses having a temporal frequency of about 100 Hz can be used to activate the C1V1-E122/E162 mutant chimeric protein. In some embodiments, activation of the C1V1-E122/E162 mutant chimeric protein with light pulses having a frequency of 100 Hz can cause depolarization-induced synaptic depletion of the neurons expressing the C1V1-E122/E162 mutant chimeric protein.

In some cases, the C1V1 variant polypeptide comprises a membrane trafficking signal and/or an ER export signal. In some embodiments, the trafficking signal can be derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

### C1C2 Chimeric Cation Channels

In other embodiments, the light-activated cation channel protein is a C1C2 chimeric protein derived from the ChR1 and the ChR2 proteins from Chlamydomonas reinhardtii, wherein the protein is responsive to light and is capable of mediating a depolarizing current in the cell when the cell is illuminated with light. In another embodiment, the light-activated polypeptide comprises an amino acid sequence at least 75%, 85%, 90%, 91%, 92%, 93%, 94%, 95%, 96%, 97%, 98%, 99%, or 100% identical to the sequence shown in SEQ ID NO:9. The light-activated cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-activated cation channel protein to regulate the polarization state of the plasma membrane of the cell. Additionally, the light-activated cation channel protein comprises one or more conservative amino acid substitutions and/or one or more non-conservative amino acid substitutions. The light-activated proton pump protein containing substitutions, deletions, and/or insertions introduced into the native amino acid sequence suitably retains the ability to transport cations across a cell membrane.

In some embodiments, a C1C2 protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the C1C2 protein comprises an N-terminal signal peptide and a C-terminal ER export signal. In some embodiments, the C1C2 protein comprises an N-terminal signal peptide and a C-terminal trafficking signal. In some embodiments, the C1C2 protein comprises an N-terminal signal peptide, a C-terminal ER export signal, and a C-terminal trafficking signal. In some embodiments, the C1C2 protein comprises a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the C1C2 protein comprises an amino acid sequence that is at least 75%, 85%, 90%, 91%, 92%, 93%, 94%, 95%, 96%, 97%, 98%, 99%, or 100% identical to the sequence shown in SEQ ID NO:10.

### ReaChR

In some aspects, a depolarizing light-activated polypeptide is a red shifted variant of a depolarizing light-activated polypeptide derived from Chlamydomonas reinhardtii; such light-activated polypeptides are referred to herein as a “ReaChR polypeptide” or “ReaChR protein” or “ReaChR.” In another embodiment, the light-activated polypeptide comprises an amino acid sequence at least 75%, 85%, 90%, 91%, 92%, 93%, 94%, 95%, 96%, 97%, 98%, 99%, or 100% identical to the sequence shown in SEQ ID NO:11. The light used to activate the ReaChR polypeptide can have a wavelength between about 590 and about 630 nm or can have a wavelength of about 610 nm. The ReaChR protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-activated cation channel protein to regulate the polarization state of the plasma membrane of the cell. Additionally, the ReaChR protein can comprise one or more conservative amino acid substitutions and/or one or more non-conservative amino acid substitutions. The ReaChR containing substitutions, deletions, and/or insertions introduced into the native amino acid sequence suitably retains the ability to transport cations across a cell membrane.

In some embodiments, a ReaChR protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the ReaChR protein comprises an N-terminal signal peptide and a C-terminal ER export signal. In some embodiments, the ReaChR protein comprises an N-terminal signal peptide and a C-terminal trafficking signal. In some embodiments, the ReaChR protein comprises an N-terminal signal peptide, a C-terminal ER export signal, and a C-terminal trafficking signal. In some embodiments, the ReaChR protein comprises a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the ReaChR protein comprises an amino acid sequence that is at least 75%, 85%, 90%, 91%, 92%, 93%, 94%, 95%, 96%, 97%, 98%, 99%, or 100% identical to the sequence shown in SEQ ID NO:12.

### SdChR

In some aspects, a depolarizing light-activated polypeptide is a SdChR polypeptide derived from Scherffelia dubia, wherein the SdChR polypeptide is capable of transporting cations across a cell membrane when the cell is illuminated with light. In some cases, the SdChR polypeptide comprises an amino acid sequence at least 75%, 85%, 90%, 91%, 92%, 93%, 94%, 95%, 96%, 97%, 98%, 99%, or 100% identical to the sequence shown in SEQ ID NO:13. The light used to activate the SdChR polypeptide can have a wavelength between about 440 and about 490 nm or can have a wavelength of about 460 nm. The SdChR protein can additionally comprise substitutions, deletions, and/or insertions introduced into a native amino acid sequence to increase or decrease sensitivity to light, increase or decrease sensitivity to particular wavelengths of light, and/or increase or decrease the ability of the SdChR protein to regulate the polarization state of the plasma membrane of the cell. In some instances, the SdChR protein comprises one or more conservative amino acid substitutions and/or one or more non-conservative amino acid substitutions. The SdChR protein containing substitutions, deletions, and/or insertions introduced into the native amino acid sequence suitably retains the ability to transport cations across a cell membrane.

In some embodiments, a SdChR protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the SdChR protein comprises an N-terminal signal peptide and a C-terminal ER export signal. In some embodiments, the SdChR protein comprises an N-terminal signal peptide and a C-terminal trafficking signal. In some embodiments, the SdChR protein comprises an N-terminal signal peptide, a C-terminal ER export signal, and a C-terminal trafficking signal. In some embodiments, the SdChR protein comprises a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal can be derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the SdChR protein comprises an amino acid sequence that is at least 75%, 85%, 90%, 91%, 92%, 93%, 94%, 95%, 96%, 97%, 98%, 99%, or 100% identical to the sequence shown in SEQ ID NO:14.

### CnChR1

In some aspects, a depolarizing light-activated polypeptide can be, e.g. CnChR1, derived from Chlamydomonas noctigama, wherein the CnChR1 polypeptide is capable of transporting cations across a cell membrane when the cell is illuminated with light. In some cases, the CnChR1 polypeptide comprises an amino acid sequence at least 75%, 85%, 90%, 91%, 92%, 93%, 94%, 95%, 96%, 97%, 98%, 99%, or 100% identical to the sequence shown in SEQ ID NO:15. The light used to activate the CnChR1 polypeptide can have a wavelength between about 560 and about 630 nm or can have a wavelength of about 600 nm. The CnChR1 protein can additionally comprise substitutions, deletions, and/or insertions introduced into a native amino acid sequence to increase or decrease sensitivity to light, increase or decrease sensitivity to particular wavelengths of light, and/or increase or decrease the ability of the CnChR1 protein to regulate the polarization state of the plasma membrane of the cell. In some cases, the CnChR1 protein comprises one or more conservative amino acid substitutions and/or one or more non-conservative amino acid substitutions. The CnChR1 protein containing substitutions, deletions, and/or insertions introduced into the native amino acid sequence suitably retains the ability to transport cations across a cell membrane.

In some embodiments, a CnChR1protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the CnChR1protein includes an N-terminal signal peptide and a C-terminal ER export signal. In some embodiments, the CnChR1protein includes an N-terminal signal peptide and a C-terminal trafficking signal. In some embodiments, the CnChR1protein comprises an N-terminal signal peptide, a C-terminal ER export signal, and a C-terminal trafficking signal. In some embodiments, the CnChR1protein comprises a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal is derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the CnChR1protein comprises an amino acid sequence that is at least 75%, 85%, 90%, 91%, 92%, 93%, 94%, 95%, 96%, 97%, 98%, 99%, or 100% identical to the sequence shown in SEQ ID NO:16.

### CsChrimson

In other embodiments, the light-activated cation channel protein is a CsChrimson chimeric protein derived from a CsChR protein of Chloromonas subdivisa and CnChR1 protein from Chlamydomonas noctigama, wherein the N terminus of the protein comprises the amino acid sequence of residues 1-73 of CsChR followed by residues 79-350 of the amino acid sequence of CnChR1; is responsive to light; and is capable of mediating a depolarizing current in the cell when the cell is illuminated with light. In another embodiment, the CsChrimson polypeptide comprises an amino acid sequence at least 75%, 85%, 90%, 91%, 92%, 93%, 94%, 95%, 96%, 97%, 98%, 99%, or 100% identical to the sequence shown in SEQ ID NO:17. The CsChrimson protein can additionally comprise substitutions, deletions, and/or insertions introduced into a native amino acid sequence to increase or decrease sensitivity to light, increase or decrease sensitivity to particular wavelengths of light, and/or increase or decrease the ability of the CsChrimson protein to regulate the polarization state of the plasma membrane of the cell. Additionally, the CsChrimson protein can comprise one or more conservative amino acid substitutions and/or one or more non-conservative amino acid substitutions. A CsChrimson protein containing substitutions, deletions, and/or insertions introduced into the native amino acid sequence suitably retains the ability to transport cations across a cell membrane.

In some embodiments, a CsChrimson protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the CsChrimson protein comprises an N-terminal signal peptide and a C-terminal ER export signal. In some embodiments, the CsChrimson protein comprises an N-terminal signal peptide and a C-terminal trafficking signal. In some embodiments, the CsChrimson protein comprises an N-terminal signal peptide, a C-terminal ER export signal, and a C-terminal trafficking signal. In some embodiments, the CsChrimson protein comprises a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal is derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the CsChrimson protein comprises an amino acid sequence that is at least 75%, 85%, 90%, 91%, 92%, 93%, 94%, 95%, 96%, 97%, 98%, 99%, or 100% identical to the sequence shown in SEQ ID NO:18.

### ShChR1

In some aspects, a depolarizing light-activated polypeptide can be, e.g. ShChR1, derived from Stigeoclonium helveticum, wherein the ShChR1 polypeptide is capable of transporting cations across a cell membrane when the cell is illuminated with light. In some cases, the ShChR1 polypeptide comprises an amino acid sequence at least 75%, 85%, 90%, 91%, 92%, 93%, 94%, 95%, 96%, 97%, 98%, 99%, or 100% identical to the sequence shown in SEQ ID NO:19. The light used to activate the ShChR1 protein derived from Stigeoclonium helveticum can have a wavelength between about 480 and about 510 nm or can have a wavelength of about 500 nm. The ShChR1 protein can additionally comprise substitutions, deletions, and/or insertions introduced into a native amino acid sequence to increase or decrease sensitivity to light, increase or decrease sensitivity to particular wavelengths of light, and/or increase or decrease the ability of the ShChR1 protein to regulate the polarization state of the plasma membrane of the cell. Additionally, the ShChR1 protein can comprise one or more conservative amino acid substitutions and/or one or more non-conservative amino acid substitutions. A ShChR1 protein containing substitutions, deletions, and/or insertions introduced into the native amino acid sequence suitably retains the ability to transport cations across a cell membrane.

In some embodiments, a ShChR1 protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the ShChR1 protein comprises an N-terminal signal peptide and a C-terminal ER export signal. In some embodiments, the ShChR1 protein comprises an N-terminal signal peptide and a C-terminal trafficking signal. In some embodiments, the ShChR1 protein comprises an N-terminal signal peptide, a C-terminal ER export signal, and a C-terminal trafficking signal. In some embodiments, the ShChR1protein comprises a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal can be derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the ShChR1 protein comprises an amino acid sequence that is at least 75%, 85%, 90%, 91%, 92%, 93%, 94%, 95%, 96%, 97%, 98%, 99%, or 100% identical to the sequence shown in SEQ ID NO:20.

Other suitable depolarizing light-activated polypeptides are described in, e.g., Klapoetke et al. Nat Methods 2014 11:338.

### Hyperpolarizing Light-Activated Polypeptides

### Arch

In some embodiments, a suitable light-activated polypeptide is an Archaerhodopsin (Arch) proton pump (e.g., a proton pump derived from Halorubrum sodomense) that can transport one or more protons across the plasma membrane of a cell when the cell is illuminated with light. The light can have a wavelength between about 530 and about 595 nm or can have a wavelength of about 560 nm. In some embodiments, the Arch protein comprises an amino acid sequence that is at least 75%, 85%, 90%, 91%, 92%, 93%, 94%, 95%, 96%, 97%, 98%, 99%, or 100% identical to the sequence shown in SEQ ID NO:21. The Arch protein can additionally have substitutions, deletions, and/or insertions introduced into a native amino acid sequence to increase or decrease sensitivity to light, increase or decrease sensitivity to particular wavelengths of light, and/or increase or decrease the ability of the Arch protein to transport ions across the plasma membrane of a neuron. Additionally, the Arch protein can comprise one or more conservative amino acid substitutions and/or one or more non-conservative amino acid substitutions. An Arch protein containing substitutions, deletions, and/or insertions introduced into the native amino acid sequence suitably retains the ability to transport ions across the plasma membrane of a neuron in response to light.

In some embodiments, the Arch protein comprises at least one (such as one, two, three, or more) amino acid sequence motifs selected from a signal peptide, an ER export signal, and a membrane trafficking signal, that enhance transport to the plasma membranes of neurons. In some embodiments, the Arch protein comprises an N-terminal signal peptide and a C-terminal ER export signal. In some embodiments, the Arch protein comprises an N-terminal signal peptide and a C-terminal trafficking signal. In some embodiments, the Arch protein comprises an N-terminal signal peptide, a C-terminal ER export signal, and a C-terminal trafficking signal. In some embodiments, the Arch protein includes a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The linker may further include a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some embodiments, the trafficking signal is derived from the amino acid sequence of the human inward rectifier potassium channel Kir2.1. In other embodiments, the trafficking signal can include the amino acid sequence KSRITSEGEYIPLDQIDINV (SEQ ID NO:56). Trafficking sequences that are suitable for use can include an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the Arch protein comprises an amino acid sequence that is at least 75%, 85%, 90%, 91%, 92%, 93%, 94%, 95%, 96%, 97%, 98%, 99%, or 100% identical to the sequence shown in SEQ ID NO:22.

### ArchT

In some embodiments, a suitable light-activated protein is an Archaerhodopsin (ArchT) proton pump (e.g., a proton pump derived from Halorubrum sp. TP009) that can transport one or more protons across the plasma membrane of a cell when the cell is illuminated with light. The light can have a wavelength between about 530 and about 595 nm or can have a wavelength of about 560 nm. In some embodiments, the ArchT protein comprises an amino acid sequence that is at least 75%, 85%, 90%, 91%, 92%, 93%, 94%, 95%, 96%, 97%, 98%, 99%, or 100% identical to the sequence shown in SEQ ID NO:23 (ArchT). The ArchT protein can additionally comprise substitutions, deletions, and/or insertions introduced into a native amino acid sequence to increase or decrease sensitivity to light, increase or decrease sensitivity to particular wavelengths of light, and/or increase or decrease the ability of the ArchT protein to transport ions across the plasma membrane of a neuron. Additionally, the ArchT protein can comprise one or more conservative amino acid substitutions and/or one or more non-conservative amino acid substitutions. The ArchT protein containing substitutions, deletions, and/or insertions introduced into the native amino acid sequence suitably retains the ability to transport ions across the plasma membrane of a neuron in response to light.

In some cases, the ArchT polypeptide comprises a membrane trafficking signal and/or an ER export signal. In some embodiments, the trafficking signal can be derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the ArchT protein comprises an amino acid sequence that is at least 75%, 85%, 90%, 91%, 92%, 93%, 94%, 95%, 96%, 97%, 98%, 99%, or 100% identical to the sequence shown in SEQ ID NO:24.

### GtR3

In some embodiments, the light-activated polypeptide is responsive to blue light and is a proton pump protein derived from Guillardia theta, wherein the proton pump protein is capable of mediating a hyperpolarizing current in the cell when the cell is illuminated with blue light; such a protein is referred to herein as a “GtR3 protein” or a “GtR3 polypeptide”. The light can have a wavelength between about 450 and about 495 nm or can have a wavelength of about 490 nm. In some embodiment, a GtR3 protein comprises an amino acid sequence at least 85%, 90%, 91%, 92%, 93%, 94%, 95%, 96%, 97%, 98%, 99%, or 100% identical to the sequence shown in SEQ ID NO:25 (GtR3). The GtR3 protein can additionally comprise substitutions, deletions, and/or insertions introduced into a native amino acid sequence to increase or decrease sensitivity to light, increase or decrease sensitivity to particular wavelengths of light, and/or increase or decrease the ability of the GtR3 protein to regulate the polarization state of the plasma membrane of the cell. Additionally, the GtR3 protein can comprise one or more conservative amino acid substitutions and/or one or more non-conservative amino acid substitutions. The GtR3 protein containing substitutions, deletions, and/or insertions introduced into the native amino acid sequence suitably retains the ability to hyperpolarize the plasma membrane of a neuronal cell in response to light.

In some cases, a GtR3 protein comprises a core amino acid sequence at least 75%, 85%, 90%, 91%, 92%, 93%, 94%, 95%, 96%, 97%, 98%, 99%, or 100% identical to the sequence shown in SEQ ID NO:25 and at least one (such as one, two, three, or more) amino acid sequence motifs which enhance transport to the plasma membranes of mammalian cells selected from the group consisting of a signal peptide, an ER export signal, and a membrane trafficking signal. In some embodiments, GtR3 protein comprises an N-terminal signal peptide and a C-terminal ER export signal. In some embodiments, the GtR3 protein comprises an N-terminal signal peptide and a C-terminal trafficking signal. In some embodiments, the light-activated proton pump protein comprises an N-terminal signal peptide, a C-terminal ER Export signal, and a C-terminal trafficking signal. In some embodiments, the GtR3 protein comprises a C-terminal ER Export signal and a C-terminal trafficking signal. In some embodiments, the signal peptide comprises the amino acid sequence MDYGGALSAVGRELLFVTNPVVVNGS (SEQ ID NO:59). In some embodiments, the first 19 amino acids are replaced with MDYGGALSAVGRELLFVTNPVVVNGS (SEQ ID NO:59). In some embodiments, the C-terminal ER Export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The GtR3 protein may further comprise a fluorescent protein, for example, but not limited to, a yellow fluorescent protein, a red fluorescent protein, a green fluorescent protein, or a cyan fluorescent protein. In some embodiments the ER Export signal is more C-terminally located than the trafficking signal. In some embodiments the trafficking signal is more C-terminally located than the ER Export signal.

In some embodiments, the trafficking signal is derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, a GtR3 protein comprises an amino acid sequence that is at least 75%, 85%, 90%, 91%, 92%, 93%, 94%, 95%, 96%, 97%, 98%, 99%, or 100% identical to the sequence shown in SEQ ID NO:26.

### Oxy

In some embodiments, a light-activated protein is an Oxyrrhis marina (Oxy) proton pump that can transport one or more protons across the plasma membrane of a cell when the cell is illuminated with light. The light can have a wavelength between about 500 and about 560 nm or can have a wavelength of about 530 nm. In some embodiments, the Oxy protein comprises an amino acid sequence that is at least 75%, 85%, 90%, 91%, 92%, 93%, 94%, 95%, 96%, 97%, 98%, 99%, or 100% identical to the sequence shown in SEQ ID NO:27. The Oxy protein can additionally comprise substitutions, deletions, and/or insertions introduced into a native amino acid sequence to increase or decrease sensitivity to light, increase or decrease sensitivity to particular wavelengths of light, and/or increase or decrease the ability of the Oxy protein to transport ions across the plasma membrane of a neuron. Additionally, the Oxy protein can comprise one or more conservative amino acid substitutions and/or one or more non-conservative amino acid substitutions. The Oxy protein containing substitutions, deletions, and/or insertions introduced into the native amino acid sequence suitably retains the ability to transport ions across the plasma membrane of a neuron in response to light.

In some embodiments, an Oxy protein comprises at least one (such as one, two, three, or more) amino acid sequence motifs that enhance transport to the plasma membranes of neurons selected from the group consisting of a signal peptide, an ER export signal, and a membrane trafficking signal. In some embodiments, the Oxy protein comprises an N-terminal signal peptide and a C-terminal ER export signal. In some embodiments, the Oxy protein includes an N-terminal signal peptide and a C-terminal trafficking signal. In some embodiments, the Oxy protein comprises an N-terminal signal peptide, a C-terminal ER export signal, and a C-terminal trafficking signal. In some embodiments, the Oxy protein comprises a C-terminal ER export signal and a C-terminal trafficking signal. In some embodiments, the C-terminal ER export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The Oxy protein may further comprise a fluorescent protein, for example, but not limited to, a yellow fluorescent protein, a red fluorescent protein, a green fluorescent protein, or a cyan fluorescent protein. In some embodiments the ER export signal is more C-terminally located than the trafficking signal. In some embodiments the trafficking signal is more C-terminally located than the ER Export signal.

In some cases, the Oxy polypeptide comprises a membrane trafficking signal and/or an ER export signal. In some embodiments, the trafficking signal can be derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the Oxy protein comprises an amino acid sequence that is at least 75%, 85%, 90%, 91%, 92%, 93%, 94%, 95%, 96%, 97%, 98%, 99%, or 100% identical to the sequence shown in SEQ ID NO:28.

### Mac

In some embodiments, the light-activated proton pump protein (referred to herein as “Mac protein”) is responsive to light and is derived from Leptosphaeria maculans, wherein the Mac proton pump protein is capable of pumping protons across the membrane of a cell when the cell is illuminated with 520 nm to 560 nm light. The light can have a wavelength between about 520 nm to about 560 nm. In some cases, a Mac protein comprises an amino acid sequence at least 75%, 85%, 90%, 91%, 92%, 93%, 94%, 95%, 96%, 97%, 98%, 99%, or 100% identical to the sequence shown in SEQ ID NO:29 or SEQ ID NO:30 (Mac; Mac 3.0). The Mac protein can additionally comprise substitutions, deletions, and/or insertions introduced into a native amino acid sequence to increase or decrease sensitivity to light, increase or decrease sensitivity to particular wavelengths of light, and/or increase or decrease the ability of the Mac protein to regulate the polarization state of the plasma membrane of the cell. Additionally, the Mac protein can comprise one or more conservative amino acid substitutions and/or one or more non-conservative amino acid substitutions. A Mac protein containing substitutions, deletions, and/or insertions introduced into the native amino acid sequence suitably retains the ability to pump protons across the plasma membrane of a neuronal cell in response to light.

In other aspects, a Mac protein comprises a core amino acid sequence at least 75%, 85%, 90%, 91%, 92%, 93%, 94%, 95%, 96%, 97%, 98%, 99%, or 100% identical to the sequence shown in SEQ ID NO:29 and at least one (such as one, two, three, or more) amino acid sequence motifs which enhance transport to the plasma membranes of mammalian cells selected from the group consisting of a signal peptide, an ER export signal, and a membrane trafficking signal. In some embodiments, the Mac protein comprises an N-terminal signal peptide and a C-terminal ER export signal. In some embodiments, the Mac protein comprises an N-terminal signal peptide and a C-terminal trafficking signal. In some embodiments, the Mac protein comprises an N-terminal signal peptide, a C-terminal ER Export signal, and a C-terminal trafficking signal. In some embodiments, the Mac protein comprises a C-terminal ER Export signal and a C-terminal trafficking signal. In some embodiments, the C-terminal ER Export signal and the C-terminal trafficking signal are linked by a linker. The linker can comprise any of about 5, 10, 20, 30, 40, 50, 75, 100, 125, 150, 175, 200, 225, 250, 275, 300, 400, or 500 amino acids in length. The Mac protein may further comprise a fluorescent protein, for example, but not limited to, a yellow fluorescent protein, a red fluorescent protein, a green fluorescent protein, or a cyan fluorescent protein. In some embodiments the ER Export signal is more C-terminally located than the trafficking signal. In some embodiments the trafficking signal is more C-terminally located than the ER Export signal.

In some cases, the Mac polypeptide includes a membrane trafficking signal and/or an ER export signal. In some embodiments, the trafficking signal can be derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

Further disclosure related to light-activated proton pump proteins can be found in International Patent Application No. PCT/US2011/028893, the disclosure of which is hereby incorporated by reference in its entirety.

### NpHR

In some cases, a suitable light-activated chloride pump protein is derived from Natronomonas pharaonis; such a protein is referred to herein as an “NpHR protein” or an “NpHR polypeptide.” In some embodiments, the NpHR protein can be responsive to amber light as well as red light and can mediate a hyperpolarizing current in the neuron when the NpHR protein is illuminated with amber or red light. The wavelength of light that can activate the NpHR protein can be between about 580 and 630 nm. In some embodiments, the light can be at a wavelength of about 589 nm or the light can have a wavelength greater than about 630 nm (e.g. less than about 740 nm). In another embodiment, the light has a wavelength of around 630 nm. In some embodiments, the NpHR protein can hyperpolarize a neural membrane for at least about 90 minutes when exposed to a continuous pulse of light. In some embodiments, the NpHR protein comprises an amino acid sequence at least about 75%, 85%, 90%, 91%, 92%, 93%, 94%, 95%, 96%, 97%, 98%, 99%, or 100% identical to the sequence shown in SEQ ID NO:31. Additionally, the NpHR protein can comprise substitutions, deletions, and/or insertions introduced into a native amino acid sequence to increase or decrease sensitivity to light, increase or decrease sensitivity to particular wavelengths of light, and/or increase or decrease the ability of the NpHR protein to regulate the polarization state of the plasma membrane of the cell. In some embodiments, the NpHR protein comprises one or more conservative amino acid substitutions. In some embodiments, the NpHR protein comprises one or more non-conservative amino acid substitutions. A NpHR protein containing substitutions, deletions, and/or insertions introduced into the native amino acid sequence suitably retains the ability to hyperpolarize the plasma membrane of a neuronal cell in response to light.

In some cases, an NpHR protein comprises a core amino acid sequence at least about 75%, 85%, 90%, 91%, 92%, 93%, 94%, 95%, 96%, 97%, 98%, 99%, or 100% identical to the sequence shown in SEQ ID NO:31; and an endoplasmic reticulum (ER) export signal. This ER export signal can be fused to the C-terminus of the core amino acid sequence or can be fused to the N-terminus of the core amino acid sequence. In some embodiments, the ER export signal is linked to the core amino acid sequence by a linker. The linker can be any of about 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the ER export signal comprises the amino acid sequence FXYENE (SEQ ID NO:57), where X can be any amino acid. In another embodiment, the ER export signal comprises the amino acid sequence VXXSL, where X can be any amino acid. In some embodiments, the ER export signal comprises the amino acid sequence FCYENEV (SEQ ID NO:58).

Endoplasmic reticulum (ER) export sequences that are suitable for use include, e.g., VXXSL (where X is any amino acid; SEQ ID NO:52)) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

In other aspects, an NpHR protein comprises core amino acid sequence at least 75%, 85%, 90%, 91%, 92%, 93%, 94%, 95%, 96%, 97%, 98%, 99%, or 100% identical to the sequence shown in SEQ ID NO:31 and a trafficking signal (e.g., which can enhance transport of the NpHR protein to the plasma membrane). The trafficking signal may be fused to the C-terminus of the core amino acid sequence or may be fused to the N-terminus of the core amino acid sequence. In some embodiments, the trafficking signal can be linked to the core amino acid sequence by a linker, which can be any of about 5, 10, 20, 30, 40, 50, 75, 100, 125, 150, 175, 200, 225, 250, 275, 300, 400, or 500 amino acids in length. The NpHR protein may further comprise a fluorescent protein, for example, but not limited to, a yellow fluorescent protein, a red fluorescent protein, a green fluorescent protein, or a cyan fluorescent protein. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56).

In some aspects, an NpHR protein comprises a core amino acid sequence at least 75%, 85%, 90%, 91%, 92%, 93%, 94%, 95%, 96%, 97%, 98%, 99%, or 100% identical to the sequence shown in SEQ ID NO:31; and at least one (such as one, two, three, or more) amino acid sequence motifs which enhance transport to the plasma membranes of mammalian cells selected from the group consisting of an ER export signal, a signal peptide, and a membrane trafficking signal. In some embodiments, the NpHR protein includes an N-terminal signal peptide, a C-terminal ER Export signal, and a C-terminal trafficking signal. In some embodiments, the C-terminal ER Export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The NpHR protein can also further comprise a fluorescent protein, for example, but not limited to, a yellow fluorescent protein, a red fluorescent protein, a green fluorescent protein, or a cyan fluorescent protein. In some embodiments the ER Export signal can be more C-terminally located than the trafficking signal. In other embodiments the trafficking signal is more C-terminally located than the ER Export signal. In some embodiments, the signal peptide includes the amino acid sequence MTETLPPVTESAVALQAE (SEQ ID NO:62). In another embodiment, the NpHR protein includes an amino acid sequence at least 95% identical to SEQ ID NO:31. In another embodiment, the NpHR protein includes an amino acid sequence at least 95% identical to SEQ ID NO:31.

Moreover, in other aspects, an NpHR protein comprises a core amino acid sequence at least 75%, 85%, 90%, 91%, 92%, 93%, 94%, 95%, 96%, 97%, 98%, 99%, or 100% identical to the sequence shown in SEQ ID NO:31, wherein the N-terminal signal peptide of SEQ ID NO:31 is deleted or substituted. In some embodiments, other signal peptides (such as signal peptides from other opsins) can be used. The light-activated protein can further comprise an ER transport signal and/or a membrane trafficking signal described herein.

In some embodiments, the light-activated protein is an NpHR protein that comprises an amino acid sequence at least 75%, at least 85%, at least 90%, at least 95%, at least 96%, at least 97%, at least 98%, at least 99% or 100% identical to the sequence shown in SEQ ID NO:31. In some embodiments, the NpHR protein further comprises an endoplasmic reticulum (ER) export signal and/or a membrane trafficking signal. For example, the NpHR protein comprises an amino acid sequence at least 95% identical to the sequence shown in SEQ ID NO:31 and an endoplasmic reticulum (ER) export signal. In some embodiments, the amino acid sequence at least 95% identical to the sequence shown in SEQ ID NO:31 is linked to the ER export signal through a linker. In some embodiments, the ER export signal comprises the amino acid sequence FXYENE (SEQ ID NO:57), where X can be any amino acid. In another embodiment, the ER export signal comprises the amino acid sequence VXXSL, where X can be any amino acid. In some embodiments, the ER export signal comprises the amino acid sequence FCYENEV (SEQ ID NO:58). In some embodiments, the NpHR protein comprises an amino acid sequence at least 95% identical to the sequence shown in SEQ ID NO:31, an ER export signal, and a membrane trafficking signal. In other embodiments, the NpHR protein comprises, from the N-terminus to the C-terminus, the amino acid sequence at least 95% identical to the sequence shown in SEQ ID NO:31, the ER export signal, and the membrane trafficking signal. In other embodiments, the NpHR protein comprises, from the N-terminus to the C-terminus, the amino acid sequence at least 95% identical to the sequence shown in SEQ ID NO:31, the membrane trafficking signal, and the ER export signal. In some embodiments, the membrane trafficking signal is derived from the amino acid sequence of the human inward rectifier potassium channel Kir2.1. In some embodiments, the membrane trafficking signal comprises the amino acid sequence KSRITSEGEYIPLDQIDINV (SEQ ID NO:56). In some embodiments, the membrane trafficking signal is linked to the amino acid sequence at least 95% identical to the sequence shown in SEQ ID NO:31 by a linker. In some embodiments, the membrane trafficking signal is linked to the ER export signal through a linker. The linker may be any of 5, 10, 20, 30, 40, 50, 75, 100, 125, 150, 175, 200, 225, 250, 275, 300, 400, or 500 amino acids in length. The linker may further comprise a fluorescent protein, for example, but not limited to, a yellow fluorescent protein, a red fluorescent protein, a green fluorescent protein, or a cyan fluorescent protein. In some embodiments, the light-activated protein further comprises an N-terminal signal peptide.

Further disclosure related to light-activated chloride pump proteins can be found in U.S. Patent Application Publication Nos: 2009/0093403 and 2010/0145418 as well as in International Patent Application NO: PCT/US2011/028893, the disclosures of each of which are hereby incorporated by reference in their entireties.

Dunaliella salina Light-Activated Polypeptide

In some embodiments, a suitable light-activated ion channel protein is, e.g., a DsChR protein derived from Dunaliella salina, wherein the ion channel protein is capable of mediating a hyperpolarizing current in the cell when the cell is illuminated with light. The light can have a wavelength between about 470 nm and about 510 nm or can have a wavelength of about 490 nm. In some embodiments, a DsChR protein comprises an amino acid sequence at least 75%, 85%, 90%, 91%, 92%, 93%, 94%, 95%, 96%, 97%, 98%, 99%, or 100% identical to the sequence shown in SEQ ID NO:34. The DsChR protein can additionally comprise substitutions, deletions, and/or insertions introduced into a native amino acid sequence to increase or decrease sensitivity to light, increase or decrease sensitivity to particular wavelengths of light, and/or increase or decrease the ability of the DsChR protein to regulate the polarization state of the plasma membrane of the cell. Additionally, the DsChR protein can comprise one or more conservative amino acid substitutions and/or one or more non-conservative amino acid substitutions. A DsChR protein containing substitutions, deletions, and/or insertions introduced into the native amino acid sequence suitably retains the ability to transport ions across the plasma membrane of a neuronal cell in response to light.

In some case, a DsChR protein comprises a core amino acid sequence at least 75%, 85%, 90%, 91%, 92%, 93%, 94%, 95%, 96%, 97%, 98%, 99%, or 100% identical to the sequence shown in SEQ ID NO:34; and at least one (such as one, two, three, or more) amino acid sequence motifs which enhance transport to the plasma membranes of mammalian cells selected from the group consisting of a signal peptide, an ER export signal, and a membrane trafficking signal. In some embodiments, the DsChR protein comprises an N-terminal signal peptide and a C-terminal ER export signal. In some embodiments, the DsChR protein comprises an N-terminal signal peptide and a C-terminal trafficking signal. In some embodiments, the DsChR protein comprises an N-terminal signal peptide, a C-terminal ER Export signal, and a C-terminal trafficking signal. In some embodiments, the DsChR protein comprises a C-terminal ER Export signal and a C-terminal trafficking signal. In some embodiments, the C-terminal ER Export signal and the C-terminal trafficking signal are linked by a linker. The linker can be any of about 5, 10, 20, 30, 40, 50, 75, 100, 125, 150, 175, 200, 225, 250, 275, 300, 400, or 500 amino acids in length. The DsChR protein may further comprise a fluorescent protein, for example, but not limited to, a yellow fluorescent protein, a red fluorescent protein, a green fluorescent protein, or a cyan fluorescent protein. In some embodiments the ER Export signal is more C-terminally located than the trafficking signal. In some embodiments the trafficking signal is more C-terminally located than the ER Export signal.

In some cases, the DsChR polypeptide comprises a membrane trafficking signal and/or an ER export signal. In some embodiments, the trafficking signal is derived from the amino acid sequence of the human inward rectifier potassium channel Kir2.1. In other embodiments, the trafficking signal comprises the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)). In some cases, the ER export signal is, e.g., VXXSL (where X is any amino acid; SEQ ID NO:52) (e.g., VKESL (SEQ ID NO:53), VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like.

In certain embodiments, the DsChR protein comprises an amino acid sequence that is at least 75%, 85%, 90%, 91%, 92%, 93%, 94%, 95%, 96%, 97%, 98%, 99%, or 100% identical to the sequence shown in SEQ ID NO:35.

### Anion Channel Polypeptides Based on C1C2

In some embodiments, a light-activated anion channel polypeptide is a C1C2 protein. In some embodiments, a C1C2 polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:36. In some embodiments, the amino acid sequence of the C1C2 protein is modified by introducing one or more of the following mutations into the amino acid sequence: T98S, E129S, E140S, E162S, V156K, H173R, T285N, V281K and/or N297Q. In some embodiments, a C1C2 protein comprises the amino acid sequence of the protein C1C2 with all 9 of the above-listed amino acid substitutions, such that the amino acid sequence of the C1C2 polypeptide is that set forth in SEQ ID NO:36.

In some embodiments, a C1C2 polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:36; and includes 1, 2, 3, 4, 5, 6, 7, 8, or 9 amino acid substitutions selected from T98S, E129S, E140S, E162S, V156K, H173R, T285N, V281K and/or N297Q, relative to the amino acid sequence of C1C2 (SEQ ID NO:36). In some embodiments, a C1C2 polypeptide includes an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:36; and includes T98S, E129S, E140S, E162S, and T285N substitutions relative to the amino acid sequence of C1C2. In some embodiments, a C1C2 polypeptide includes an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:36; and includes V156K, H173R, V281K, and N297Q substitutions relative to the amino acid sequence of C1C2.

In some embodiments, a C1C2 polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:36; and includes 1, 2, 3, 4, 5, 6, 7, 8, or 9 of: S98, S129, S140, S162, K156, R173, N285, K281, and Q297, where the amino acid numbering is as set forth in SEQ ID NO:36. In some embodiments, a C1C2 polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:36; and includes S98, S129, S140, S162, K156, R173, N285, K281, and Q297, where the amino acid numbering is as set forth in SEQ ID NO:36. In any one of these embodiments, a C1C2 polypeptide can comprise a membrane trafficking signal (e.g., KSRITSEGEYIPLDQIDINV (SEQ ID NO:56)). In any one of these embodiments, a C1C2 polypeptide can comprise an ER export signal (e.g., FCYENEV (SEQ ID NO:58)). In any one of these embodiments, a C1C2 polypeptide comprises both a membrane trafficking signal (e.g., KSRITSEGEYIPLDQIDINV (SEQ ID NO:56)) and an ER export signal (e.g., FCYENEV (SEQ ID NO:58)). Thus, in certain embodiments, the C1C2 protein comprises an amino acid sequence that is at least 75%, 85%, 90%, 91%, 92%, 93%, 94%, 95%, 96%, 97%, 98%, 99%, or 100% identical to the sequence shown in SEQ ID NO:36.

In some embodiments, a C1C2 polypeptide is based on the amino acid sequence of the protein C1C2 (SEQ ID NO:36), wherein the amino acid sequence has been modified by replacing the first 50 N-terminal amino acids of C1C2 with amino acids 1-11 from the protein ChR2 (MDYGGALSAVG) (SEQ ID NO:63). In some embodiments, a suitable light-activated anion channel polypeptide is referred to as “ibC1C2” and comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:40; and includes 1, 2, 3, 4, 5, 6, 7, 8, or 9 of: S59, S90, S101, S123, K117, R134, N246, K242, and Q258, where the amino acid numbering is as set forth in SEQ ID NO:40. In some embodiments, a suitable light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:40; and includes S59, S90, S101, S123, K117, R134, N246, K242, and Q258, where the amino acid numbering is as set forth in SEQ ID NO:40. In some embodiments, a suitable light-activated anion channel polypeptide comprises the amino acid sequence set forth in SEQ ID NO:40. In any one of these embodiments, a suitable anion channel polypeptide comprises a membrane trafficking signal (e.g., KSRITSEGEYIPLDQIDINV (SEQ ID NO:56)). In any one of these embodiments, a suitable anion channel polypeptide comprises an ER export signal (e.g., FCYENEV (SEQ ID NO:58)). In any one of these embodiments, a suitable anion channel polypeptide comprises both a membrane trafficking signal (e.g., KSRITSEGEYIPLDQIDINV (SEQ ID NO:56)) and an ER export signal (e.g., FCYENEV (SEQ ID NO:58)). Thus, in certain embodiments, the ibC1C2 protein comprises an amino acid sequence that is at least 75%, 85%, 90%, 91%, 92%, 93%, 94%, 95%, 96%, 97%, 98%, 99%, or 100% identical to the sequence shown in SEQ ID NO:40.

In some embodiments, a suitable light-activated anion channel polypeptide is based on the amino acid sequence of the protein C1C2 (SEQ ID NO:36), wherein the cysteine amino acid residue at position 167 has been replaced by a threonine residue. In some embodiments, a suitable light-activated anion channel polypeptide, e.g., SwiChR_{CT}, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:38; and comprises 1, 2, 3, 4, 5, 6, 7, 8, or 9 of: S98, S129, S140, S162, K156, R173, N285, K281, and Q297; and includes T167. In some embodiments, a suitable light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:38; and includes S98, S129, S140, S162, K156, R173, N285, K281, and Q297; and includes T167, where the amino acid numbering is as set forth in SEQ ID NO:38. In some embodiments, a light-activated anion channel polypeptide comprises the amino acid sequence provided in SEQ ID NO:38. In some of these embodiments, the light-activated polypeptide exhibits prolonged stability of photocurrents. In some embodiments, the first 50 amino acids are replaced with MDYGGALSAVG (SEQ ID NO:63). In any one of these embodiments, a suitable anion channel polypeptide comprises a membrane trafficking signal (e.g., KSRITSEGEYIPLDQIDINV (SEQ ID NO:56)). In any one of these embodiments, a suitable anion channel polypeptide comprises an ER export signal (e.g., FCYENEV (SEQ ID NO:58)). In any one of these embodiments, a suitable anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a suitable light-activated anion channel polypeptide is based on the amino acid sequence of the protein C1C2, wherein the cysteine amino acid residue at position 167 has been replaced by an alanine residue. In some embodiments, a suitable light-activated anion channel polypeptide, SwiChR_{CA}, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:38; and includes 1, 2, 3, 4, 5, 6, 7, 8, or 9 of: S98, S129, S140, S162, K156, R173, N285, K281, and Q297; and includes A167, where the amino acid numbering is as set forth in SEQ ID NO:38. In some embodiments, a suitable light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:38; and includes S98, S129, S140, S162, K156, R173, N285, K281, and Q297; and includes A167, where the amino acid numbering is as set forth in SEQ ID NO:38. In some embodiments, the first 50 amino acids are replaced with MDYGGALSAVG (SEQ ID NO:63). In any one of these embodiments, a suitable anion channel polypeptide comprises a membrane trafficking signal (e.g., KSRITSEGEYIPLDQIDINV (SEQ ID NO:56)). In any one of these embodiments, a subject anion channel polypeptide includes an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a suitable light-activated anion channel polypeptide is based on the amino acid sequence of the protein C1C2, wherein the cysteine amino acid residue at position 167 has been replaced by a serine residue. In some embodiments, a suitable light-activated anion channel polypeptide, SwiChR_{CS}, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:38; and includes 1, 2, 3, 4, 5, 6, 7, 8, or 9 of: S98, S129, S140, S162, K156, R173, N285, K281, and Q297; and includes S167, where the amino acid numbering is as set forth in SEQ ID NO:38. In some embodiments, a suitable light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:38; and includes S98, S129, S140, S162, K156, R173, N285, K281, and Q297; and includes S167, where the amino acid numbering is as set forth in SEQ ID NO:38. In some embodiments, the first 50 amino acids are replaced with MDYGGALSAVG (SEQ ID NO:63). In any one of these embodiments, a suitable anion channel polypeptide comprises a membrane trafficking signal (e.g., KSRITSEGEYIPLDQIDINV (SEQ ID NO:56)). In any one of these embodiments, a subject anion channel polypeptide includes an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In certain embodiments, the SwiChR protein comprises an amino acid sequence that is at least 75%, 85%, 90%, 91%, 92%, 93%, 94%, 95%, 96%, 97%, 98%, 99%, or 100% identical to the sequence shown in SEQ ID NO:39.

In some embodiments, a suitable light-activated anion channel polypeptide, SwiChR, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:38; and includes 1, 2, 3, 4, 5, 6, 7, 8, or 9 of: S98, S129, S140, S162, K156, R173, N285, K281, and Q297; includes N195, or A195; and includes A167, where the amino acid numbering is as set forth in SEQ ID NO:38. In some embodiments, a suitable light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:38; and includes S98, S129, S140, S162, K156, R173, N285, K281, and Q297; includes A167; and includes N195, or A195, where the amino acid numbering is as set forth in SEQ ID NO:38. In some embodiments, the first 50 amino acids are replaced with MDYGGALSAVG (SEQ ID NO:63). In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a suitable light-activated anion channel polypeptide is based on the amino acid sequence of the protein C1C2 with one or more of the modifications described above, wherein the aspartate amino acid residue at original position 195 has been replaced by an alanine residue. In certain embodiments wherein the first 50 N-terminal amino acids of the protein are replaced by amino acids 1-11 from the protein ChR2, the aspartate amino acid residue at position 156 (which corresponds to original position 195 of the C1C2 amino acid sequence set forth in SEQ ID NO:36) is replaced by an alanine residue.

In some embodiments, a suitable hyperpolarizing light-activated polypeptide is based on the amino acid sequence of the protein C1C2 with one or more of the modifications described above, wherein the aspartate amino acid residue at original position 195 has been replaced by an asparagine residue. In certain embodiments wherein the first 50 N-terminal amino acids of the protein are replaced by amino acids 1-11 from the protein ChR2, the aspartate amino acid residue at position 156 (which corresponds to original position 195 of the C1C2 amino acid sequence set forth in SEQ ID NO:36) is replaced by an asparagine residue.

In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:40; and includes 1, 2, 3, 4, 5, 6, 7, 8, or 9 of: S59, S90, S101, S123, K117, R134, N246, K242, and Q258; and includes A128, T128 or S128, where the amino acid numbering is as set forth in SEQ ID NO:40. In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:40; and includes S59, S90, S101, S123, K117, R134, N246, K242, and Q258; and includes A128, T128 or S128, where the amino acid numbering is as set forth in SEQ ID NO:40. In any one of these embodiments, a subject anion channel polypeptide comprises a membrane trafficking signal (e.g., KSRITSEGEYIPLDQIDINV (SEQ ID NO:56)). In any one of these embodiments, a suitable anion channel polypeptide comprises an ER export signal (e.g., FCYENEV (SEQ ID NO:58)). In any one of these embodiments, a suitable anion channel polypeptide includes both a membrane trafficking signal (e.g., KSRITSEGEYIPLDQIDINV (SEQ ID NO:56)) and an ER export signal (e.g., FCYENEV (SEQ ID NO:58)).

### Anion Channel Proteins Based on ChR2

In some embodiments, a suitable hyperpolarizing light-activated polypeptide is based on the amino acid sequence of the protein ChR2. The amino acid sequence of ChR2 is set forth in SEQ ID NO:42. In some embodiments, the amino acid sequence of the ChR2 protein has been modified by introducing one or more of the following mutations into the amino acid sequence: A59S, E90S, E101S, E123S, Q117K, H134R, V242K, T246N and/or N258Q. In some embodiments, a suitable hyperpolarizing light-activated polypeptide comprises the amino acid sequence of the protein ChR2 with all 9 of the above-listed amino acid substitutions, such that the amino acid sequence of the polypeptide is provided in SEQ ID NO:42 (iChR2).

In some embodiments, a suitable light-activated anion channel polypeptide iChR2 comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:42; and includes 1, 2, 3, 4, 5, 6, 7, 8, or 9 amino acid substitutions selected from A59S, E90S, E101S, E123S, Q117K, H134R, V242K, T246N and/or N258Q, relative to the amino acid sequence of ChR2 (SEQ ID NO:1).

In some embodiments, a suitable light-activated polypeptide (“iChR2”) comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:42; and includes 1, 2, 3, 4, 5, 6, 7, 8, or 9 of: S59, S90, S101, S123, K117, R134, K242, N246 and Q258, where the amino acid numbering is as set forth in SEQ ID NO:42. In some embodiments, an iChR2 polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:42; and includes 1, 2, 3, 4, 5, 6, 7, 8, 9, 10 or 11 of: S59, S90, S101, S123, K117, R134, K242, N246, Q258, and either N156 or A156, and either T128, A128, or S128, where the amino acid numbering is as set forth in SEQ ID NO:42. In some embodiments, an iChR2 polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:42; and includes S59, S90, S101, S123, K117, R134, K242, N246 and Q258, where the amino acid numbering is as set forth in SEQ ID NO:42. In any one of these embodiments, an iChR2 polypeptide can comprise a membrane trafficking signal (e.g., KSRITSEGEYIPLDQIDINV (SEQ ID NO:56)). In any one of these embodiments, an iChR2 polypeptide can comprise an ER export signal (e.g., FCYENEV (SEQ ID NO:58)). In any one of these embodiments, an iChR2 polypeptide can comprise both a membrane trafficking signal (e.g., KSRITSEGEYIPLDQIDINV (SEQ ID NO:56)) and an ER export signal (e.g., FCYENEV (SEQ ID NO:58)). Thus in certain embodiments, the iChR2 protein comprises an amino acid sequence that is at least 75%, 85%, 90%, 91%, 92%, 93%, 94%, 95%, 96%, 97%, 98%, 99%, or 100% identical to the sequence shown in SEQ ID NO:43.

### Anion Channel Polypeptides Based on C1V1

In some embodiments, a suitable hyperpolarizing light-activated polypeptide is based on the amino acid sequence of the protein C1V1. The amino acid sequence of C1V1 is set forth in SEQ ID NO:44. In some embodiments, the amino acid sequence of the C1V1 protein has been modified by introducing one or more of the following mutations into the amino acid sequence: T98S, E129S, E140S, E162S, V156K, H173R, A285N, P281K and/or N297Q. In some embodiments, a hyperpolarizing light-activated polypeptide comprises the amino acid sequence of the protein C1V1 with all 9 of the above-listed amino acid substitutions, such that the amino acid sequence of the polypeptide is provided in SEQ ID NO:44.

In some embodiments, a suitable light-activated anion channel polypeptide, iC1V1, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:44; and includes 1, 2, 3, 4, 5, 6, 7, 8, or 9 amino acid substitutions selected from T98S, E129S, E140S, E162S, V156K, H173R, A285N, P281K and/or N297Q, relative to the amino acid sequence of C1V1 (SEQ ID NO:7).

In some embodiments, a suitable light-activated anion channel polypeptide, iC1V1, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:44; and includes 1, 2, 3, 4, 5, 6, 7, 8, or 9 of: S98, S129, S140, S162, K156, R173, N285, K281, and Q297, where the amino acid numbering is as set forth in SEQ ID NO:44. In some embodiments, a suitable light-activated anion channel polypeptide (referred to as “iC1V1”), comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:44; and includes 1, 2, 3, 4, 5, 6, 7, 8, or 9 of: S98, S129, S140, S162, K156, R173, N285, K281, and Q297, and includes N195, where the amino acid numbering is as set forth in SEQ ID NO:44. In some embodiments, a suitable light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:44; and includes S98, S129, S140, S162, K156, R173, N285, K281, and Q297, where the amino acid numbering is as set forth in SEQ ID NO:44. In any one of these embodiments, a suitable anion channel polypeptide includes a membrane trafficking signal (e.g., KSRITSEGEYIPLDQIDINV (SEQ ID NO:56)). In any one of these embodiments, a subject anion channel polypeptide includes an ER export signal (e.g., FCYENEV (SEQ ID NO:58)). In any one of these embodiments, a suitable anion channel polypeptide comprises both a membrane trafficking signal (e.g., KSRITSEGEYIPLDQIDINV (SEQ ID NO:56)) and an ER export signal (e.g., FCYENEV (SEQ ID NO:58)). Thus in certain embodiments, the iC1V1protein can have an amino acid sequence that is at least 75%, 85%, 90%, 91%, 92%, 93%, 94%, 95%, 96%, 97%, 98%, 99%, or 100% identical to the sequence shown in SEQ ID NO:45.

In some embodiments, a suitable hyperpolarizing light-activated polypeptide is based on the amino acid sequence of the protein C1V1 (SEQ ID NO:7), wherein the amino acid sequence has been modified by replacing the first 50 N-terminal amino acids of C1V1 with amino acids 1-11 from the protein ChR2 (MDYGGALSAVG) (SEQ ID NO:63). In some embodiments, a suitable hyperpolarizing light-activated polypeptide, ibC1V1, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:46; and includes 1, 2, 3, 4, 5, 6, 7, 8, or 9 of: S59, S90, S101, S123, K117, R134, N246, K242, and Q258, where the amino acid numbering is as set forth in SEQ ID NO:46. In some embodiments, a suitable hyperpolarizing light-activated polypeptide (referred to as “ibC1V1”), comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:46; and includes 1, 2, 3, 4, 5, 6, 7, 8, or 9 of: S59, S90, S101, S123, K117, R134, N246, K242, and Q258, and includes N156, where the amino acid numbering is as set forth in SEQ ID NO:46. In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:46; and includes S59, S90, S101, S123, K117, R134, N246, K242, and Q258, where the amino acid numbering is as set forth in SEQ ID NO:46. In some embodiments, a suitable light-activated anion channel polypeptide comprises the amino acid sequence set forth in SEQ ID NO:46. In any one of these embodiments, a suitable anion channel polypeptide comprises a membrane trafficking signal (e.g., KSRITSEGEYIPLDQIDINV (SEQ ID NO:56)). In any one of these embodiments, a suitable anion channel polypeptide comprises an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)). Thus in certain embodiments, an ibC1V1 protein comprises an amino acid sequence that is at least 75%, 85%, 90%, 91%, 92%, 93%, 94%, 95%, 96%, 97%, 98%, 99%, or 100% identical to the sequence shown in SEQ ID NO:47.

In some embodiments, a suitable hyperpolarizing light-activated polypeptide is based on the amino acid sequence of the protein C1V1 (SEQ ID NO:7), wherein the cysteine amino acid residue at position 167 has been replaced by a threonine residue. In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:7; and includes 1, 2, 3, 4, 5, 6, 7, 8, or 9 of: S98, S129, S140, S162, K156, R173, N285, K281, and Q297; and includes T167. In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:44; and includes S98, S129, S140, S162, K156, R173, N285, K281, and Q297; and includes T167, S167 or A167, where the amino acid numbering is as set forth in SEQ ID NO:44. In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:46; and includes S98, S129, S140, S162, K156, R173, N285, K281, and Q297; includes T167, S167 or A167; and includes A195 or N195, where the amino acid numbering is as set forth in SEQ ID NO:46. In some embodiments, the first 50 amino acids are replaced with MDYGGALSAVG (SEQ ID NO:63). In any one of these embodiments, a suitable hyperpolarizing light-activated polypeptide comprises a membrane trafficking signal (e.g., KSRITSEGEYIPLDQIDINV (SEQ ID NO:56)). In any one of these embodiments, a suitable hyperpolarizing light-activated polypeptidecomprises an ER export signal (e.g., FCYENEV (SEQ ID NO:58)). In any one of these embodiments, a suitable hyperpolarizing light-activated polypeptide includes both a membrane trafficking signal (e.g., KSRITSEGEYIPLDQIDINV (SEQ ID NO:56)) and an ER export signal (e.g., FCYENEV (SEQ ID NO:58)).

In some embodiments, a suitable hyperpolarizing light-activated polypeptide is based on the amino acid sequence of the protein C1V1 with one or more of the modifications described above, wherein the aspartate amino acid residue at original position 195 has been replaced by an alanine residue. In certain embodiments wherein the first 50 N-terminal amino acids of the protein are replaced by amino acids 1-11 from the protein ChR2, the aspartate amino acid residue at position 156 (which corresponds to original position 195 of the C1V1 amino acid sequence set forth in SEQ ID NO:7) is replaced by an alanine residue.

In some embodiments, a suitable hyperpolarizing light-activated polypeptide is based on the amino acid sequence of the protein C1V1 with one or more of the modifications described above, wherein the aspartate amino acid residue at original position 195 has been replaced by an asparagine residue. In certain embodiments wherein the first 50 N-terminal amino acids of the protein are replaced by amino acids 1-11 from the protein ChR2, the aspartate amino acid residue at position 156 (which corresponds to original position 195 of the C1V1 amino acid sequence set forth in SEQ ID NO:7) is replaced by an asparagine residue.

In some embodiments, a suitable hyperpolarizing light-activated polypeptide, ibC1V1, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:46; and includes 1, 2, 3, 4, 5, 6, 7, 8, or 9 of: S59, S90, S101, S123, K117, R134, N246, K242, and Q258; and includes T128, A128, or S128, where the amino acid numbering is as set forth in SEQ ID NO:46. In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:46; and includes S59, S90, S101, S123, K117, R134, N246, K242, and Q258; and includes T128, A128, or S128, where the amino acid numbering is as set forth in SEQ ID NO:46. In any one of these embodiments, a suitable anion channel polypeptide comprises a membrane trafficking signal (e.g., KSRITSEGEYIPLDQIDINV (SEQ ID NO:56)). In any one of these embodiments, a suitable anion channel polypeptide comprises an ER export signal (e.g., FCYENEV (SEQ ID NO:58)). In any one of these embodiments, a suitable anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:46; and includes 1, 2, 3, 4, 5, 6, 7, 8, or 9 of: S59, S90, S101, S123, K117, R134, N246, K242, and Q258; and includes T128, A128, or S128; and includes A156 or N156, where the amino acid numbering is as set forth in SEQ ID NO:46. In some embodiments, a suitable hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:46; and includes S59, S90, S101, S123, K117, R134, N246, K242, and Q258; and includes T128, A128, or S128; and includes A156 or N156, where the amino acid numbering is as set forth in SEQ ID NO:46. In any one of these embodiments, a suitable hyperpolarizing light-activated polypeptide comprises a membrane trafficking signal (e.g., KSRITSEGEYIPLDQIDINV (SEQ ID NO:56)). In any one of these embodiments, a suitable hyperpolarizing light-activated polypeptide comprises an ER export signal (e.g., FCYENEV (SEQ ID NO:58)). In any one of these embodiments, a subject anion channel polypeptide includes both a membrane trafficking signal (e.g., KSRITSEGEYIPLDQIDINV (SEQ ID NO:56)) and an ER export signal (e.g., FCYENEV (SEQ ID NO:58)).

### Anion Channel Polypeptides Based on ReaChR

In some embodiments, a subject hyperpolarizing light-activated polypeptide is based on the amino acid sequence of the protein ReaChR. The amino acid sequence of ReaChR is set forth in SEQ ID NO:11. In some embodiments, the amino acid sequence of the ReaChR protein has been modified by introducing one or more of the following mutations into the amino acid sequence: T99S, E130S, E141S, E163S, V157K, H174R, A286N, P282K and/or N298Q. In some embodiments, a subject hyperpolarizing light-activated polypeptide comprises the amino acid sequence of the protein ReaChR with all 9 of the above-listed amino acid substitutions, such that the amino acid sequence of the polypeptide is provided in SEQ ID NO:48.

In some embodiments, a subject light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:48; and includes 1, 2, 3, 4, 5, 6, 7, 8, or 9 amino acid substitutions selected from T99S, E130S, E141S, E163S, V157K, H174R, A286N, P282K and/or N298Q, relative to the amino acid sequence of ReaChR (SEQ ID NO:11).

In some embodiments, a subject light-activated anion channel polypeptide, iReaChR, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:48; and includes 1, 2, 3, 4, 5, 6, 7, 8, or 9 of: S99, S130, S141, S163, K157, R174, N286, K281, and Q298, where the amino acid numbering is as set forth in SEQ ID NO:48. In some embodiments, a subject light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:48; and includes S99, S130, S141, S163, K157, R174, N286, K281, and Q298, where the amino acid numbering is as set forth in SEQ ID NO:48. In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide includes both a membrane trafficking signal (e.g., KSRITSEGEYIPLDQIDINV (SEQ ID NO:56)) and an ER export signal (e.g., FCYENEV (SEQ ID NO:58)). Thus in certain embodiments, the iReaChR protein comprises an amino acid sequence that is at least 75%, 85%, 90%, 91%, 92%, 93%, 94%, 95%, 96%, 97%, 98%, 99%, or 100% identical to the sequence shown in SEQ ID NO:49.

In some embodiments, a subject light-activated anion channel polypeptide, iReaChR, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:48; and includes 1, 2, 3, 4, 5, 6, 7, 8, or 9 of: S99, S130, S141, S163, K157, R174, N286, K281, and Q298, and includes N196, where the amino acid numbering is as set forth in SEQ ID NO:48. In some embodiments, a subject light-activated anion channel polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:48; and includes S99, S130, S141, S163, K157, R174, N286, K281, and Q298, and includes N196, where the amino acid numbering is as set forth in SEQ ID NO:48. In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a subject hyperpolarizing light-activated polypeptide is based on the amino acid sequence of the protein ReaChR (SEQ ID NO:11), wherein the amino acid sequence has been modified by replacing the first 51 N-terminal amino acids of ReaChR with amino acids 1-11 from the protein ChR2 (MDYGGALSAVG) (SEQ ID NO:63). In some embodiments, a subject hyperpolarizing light-activated polypeptide, ibReaChR, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:50; and includes 1, 2, 3, 4, 5, 6, 7, 8, or 9 of: S59, S90, S101, S123, K117, R134, N246, K242, and Q258, where the amino acid numbering is as set forth in SEQ ID NO:50. In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:50; and includes S59, S90, S101, S123, K117, R134, N246, K242, and Q258, where the amino acid numbering is as set forth in SEQ ID NO:50. In some embodiments, a subject light-activated anion channel polypeptide comprises the amino acid sequence set forth in SEQ ID NO:50. In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)). Thus in certain embodiments, the ibReaChR protein can have an amino acid sequence that is at least 75%, 85%, 90%, 91%, 92%, 93%, 94%, 95%, 96%, 97%, 98%, 99%, or 100% identical to the sequence shown in SEQ ID NO:51.

In some embodiments, a subject hyperpolarizing light-activated polypeptide is based on the amino acid sequence of the protein ReaChR (SEQ ID NO:11), wherein the amino acid sequence has been modified by replacing the first 51 N-terminal amino acids of ReaChR with amino acids 1-11 from the protein ChR2 (MDYGGALSAVG) (SEQ ID NO:63). In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:11; and includes 1, 2, 3, 4, 5, 6, 7, 8, or 9 of: S59, S90, S101, S123, K117, R134, N246, K242, and Q258, and includes N156, where the amino acid numbering is as set forth in SEQ ID NO:11. In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:11; and includes S59, S90, S101, S123, K117, R134, N246, K242, and Q258, and includes N156, where the amino acid numbering is as set forth in SEQ ID NO:11. In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a subject hyperpolarizing light-activated polypeptide is based on the amino acid sequence of the protein ReaChR (SEQ ID NO:11), wherein the cysteine amino acid residue at position 168 has been replaced by a threonine residue. In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:11; and includes 1, 2, 3, 4, 5, 6, 7, 8, or 9 of: S99, S130, S141, S163, K157, R174, N286, K281, and Q298; and includes T168, S168 or A168. In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:11; and includes S99, S130, S141, S163, K157, R174, N286, K281, and Q298; and includes T168, S168 or A168, where the amino acid numbering is as set forth in SEQ ID NO:11. In some embodiments, the first 51 amino acids are replaced with MDYGGALSAVG (SEQ ID NO:63). In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a subject hyperpolarizing light-activated polypeptide, iReaChR, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:48; and includes 1, 2, 3, 4, 5, 6, 7, 8, or 9 of: S99, S130, S141, S163, K157, R174, N286, K281, and Q298; includes A196 or N196; and includes T168, S168, or A168, where the amino acid numbering is as set forth in SEQ ID NO:48. In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth SEQ ID NO:48; and includes S99, S130, S141, S163, K157, R174, N286, K281, and Q298; includes A196 or N196; and includes T168, S168, or A168, where the amino acid numbering is as set forth in SEQ ID NO:48. In some embodiments, the first 51 amino acids are replaced with MDYGGALSAVG (SEQ ID NO:63). In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide includes both a membrane trafficking signal (e.g., KSRITSEGEYIPLDQIDINV (SEQ ID NO:56)) and an ER export signal (e.g., FCYENEV (SEQ ID NO:58)).

In some embodiments, a subject hyperpolarizing light-activated polypeptide is based on the amino acid sequence of the protein ReaChR with one or more of the modifications described above, wherein the aspartate amino acid residue at original position 196 has been replaced by an alanine residue. In certain embodiments wherein the first 51 N-terminal amino acids of the protein are replaced by amino acids 1-11 from the protein ChR2, the aspartate amino acid residue at position 156 (which corresponds to original position 196 of the ReaChR amino acid sequence set forth in SEQ ID NO:11) is replaced by an alanine residue.

In some embodiments, a subject hyperpolarizing light-activated polypeptide is based on the amino acid sequence of the protein ReaChR with one or more of the modifications described above, wherein the aspartate amino acid residue at original position 196 has been replaced by an asparagine residue. In certain embodiments wherein the first 51 N-terminal amino acids of the protein are replaced by amino acids 1-11 from the protein ChR2, the aspartate amino acid residue at position 156 (which corresponds to original position 196 of the ReaChR amino acid sequence set forth in SEQ ID NO:11) is replaced by an asparagine residue.

In some embodiments, a subject hyperpolarizing light-activated polypeptide, ibReaChR, comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:50; and includes 1, 2, 3, 4, 5, 6, 7, 8, or 9 of: S59, S90, S101, S123, K117, R134, N246, K242, and Q258; and includes T128, S128 or A128, where the amino acid numbering is as set forth in SEQ ID NO:50. In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:50; and includes S59, S90, S101, S123, K117, R134, N246, K242, and Q258; and includes T128, where the amino acid numbering is as set forth in SEQ ID NO:50. In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide comprises an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

In some embodiments, a subject hyperpolarizing light-activated polypeptide, ibReaChR, includes an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:50; and includes 1, 2, 3, 4, 5, 6, 7, 8, or 9 of: S59, S90, S101, S123, K117, R134, N246, K242, and Q258; includes T128, S128 or A128; and includes A156 or N156, where the amino acid numbering is as set forth in SEQ ID NO:50. In some embodiments, a subject hyperpolarizing light-activated polypeptide comprises an amino acid sequence having at least 58%, at least 60%, at least 65%, at least 70%, at least 75%, at least 80%, at least 85%, at least 90%, at least 95%, at least 98%, at least 99%, or 100%, amino acid sequence identity to the amino acid sequence set forth in SEQ ID NO:50; and includes S59, S90, S101, S123, K117, R134, N246, K242, and Q258; includes T128, S128 or A128; and includes A156 or N156, where the amino acid numbering is as set forth in SEQ ID NO:50. In any one of these embodiments, a subject anion channel polypeptide comprises a membrane trafficking signal (e.g., KSRITSEGEYIPLDQIDINV (SEQ ID NO:56)). In any one of these embodiments, a subject anion channel polypeptide includes an ER export signal (e.g., FCYENEV (SEQ ID NO:58)). In any one of these embodiments, a subject anion channel polypeptide comprises both a membrane trafficking signal (e.g., KSRITSEGEYIPLDQIDINV (SEQ ID NO:56)) and an ER export signal (e.g., FCYENEV (SEQ ID NO:58)).

### Utility

The methods of the present disclosure find a variety of uses. As described above, the methods of the present disclosure find use in modulating temporal patterns of neuronal activity in one or more regions of the brain using fMRI, optogenetics, and/or electrophysiological recordings. In some cases, the present method may provide a way to identify new roles for anatomically and/or functionally defined neurons in functional circuits.

In some cases, the present methods identify specific circuit mechanisms underlying VLO control of brain-wide neural activities. Thalamic input to the VLO plays a key role in modulating perceived pain levels during noxious stimulus and supports goal-directed behavior by signaling predictive cues and expected outcome.

In certain embodiments, the present methods provide for selectively activating a specific population of neurons, via a combination of selective expression of light-activated polypeptides and selective illumination of brain regions, at different temporal frequencies, wherein the number of neurons activated at each frequency remains substantially the same. Thus, an effect of increased frequency of light pulses activating a first region on the response at a functionally connected second region of the brain may be attributed mainly to the change in frequency, and not on other factors, e.g., recruitment of more neurons in a frequency-dependent manner.

The present methods also find use in probing the effect of deep brain stimulation (DBS) of brain regions, e.g., the central thalamus, insula, cingulate, subthalamic nucleus (STN), globus pallidus interna (GPI), zona incerta (ZI), etc., that may find use in the treatment of various neurological disorders, such as pain, depression, addiction, Alzheimer's disease, attention deficit disorder, autism, anorgasmia, cerebral palsy, bipolar depression, unipolar depression, epilepsy, generalized anxiety disorder, acute head trauma, hedonism, obesity, obsessive-compulsive disorder (OCD), acute pain, chronic pain, Parkinson's disease, persistent vegetative state, phobia, post-traumatic stress disorder, rehabilitation/regenesis for post-stroke, post-head trauma, social anxiety disorder, Tourette's Syndrome, hemorrhagic stroke, and ischemic stroke. The present methods, in some cases, may provide a way to probe the effect of a single parameter of stimulation, such as light pulse frequency or pulse width, of a defined population of neurons, on global brain dynamics, as well as cellular level functional circuits.

### Examples of Non-Limiting Aspects of the Disclosure

1. A method for modulating temporal patterns of neuronal activity in the brain of an individual, the method comprising:

i) stimulating, with a light pulse from an optical light source, one or more of thalamocortical projections, thalamic relay neurons, cortical projection neurons, cell bodies in a thalamic submedial nucleus, and cell bodies in the ventrolateral orbitofrontal cortex (VLO) in the brain, wherein neuronal cell bodies in one or more of the VLO and a thalamus of the individual express a light-activated polypeptide; and

ii) measuring a functional magnetic resonance imaging (fMRI) signal of the whole-brain, wherein said measuring occurs during said stimulating,

wherein a positive measured fMRI signal is associated with an increase in neuronal activity following said stimulating, and wherein a negative measured fMRI signal is associated with a decrease in neuronal activity following said stimulating.

2. The method of Aspect 1, wherein the whole-brain comprises an ipsilateral and contralateral brain region.

3. The method of Aspect 2, wherein the ipsilateral region comprises a left hemisphere of the brain comprising a medial prefontal cortex, a lateral prefontal cortex, a motor cortex, a cingulate cortex, a sensory cortex, an insular cortex, a striatum, and a thalamus.

4. The method of Aspect 2, wherein the contralateral region comprises a right hemisphere of the brain comprising a medial prefontal cortex, a lateral prefontal cortex, a motor cortex, a cingulate cortex, a sensory cortex, an insular cortex, a striatum, and a thalamus.

5. The method of Aspect 1, wherein the light pulse has a frequency ranging from 5 Hz to 40 Hz.

6. The method of Aspect 5, wherein the light pulse has a frequency of 10 Hz.

7. The method of Aspect 5, wherein the light pulse has a frequency of 40 Hz.

8. The method of Aspect 1, wherein stimulating the thalamocortical projection with the light pulse having a frequency of 10 Hz or more results in the negative fMRI signal in the sensory, motor, and cingulate cortex of the ipsilateral region of the brain.

9. The method of Aspect 1, wherein stimulating the thalamocortical projection with the light pulse having a frequency of 5 Hz or more results in the negative fMRI signal in the sensory, motor, and cingulate cortex of the ipsilateral region of the brain.

10. The method of Aspect 1, wherein stimulating the thalamocortical projection with the light pulse having a frequency of 40 Hz or more results in the positive measured fMRI signal.

11. The method of Aspect 1, wherein stimulating the thalamocortical projection with the light pulse having a frequency of 5 Hz or more results in the negative measured fMRI in the contralateral region of the brain.

12. The method of Aspect 1, wherein stimulating the thalamocortical projection with the light pulse having a frequency of 10 Hz or more results in the negative measured fMRI signal in the contralateral region of the brain.

13. The method of Aspect 1, wherein stimulating the cell bodies in the VLO with the light pulse having a frequency ranging from 5 Hz to 40 Hz results in the positive measured fMRI signal in the ipsilateral region of the brain.

14. The method of Aspect 1, wherein the light-activated polypeptides are expressed in neurons of the submedial nucleus of the thalamus.

15. The method of Aspect 1, wherein the light-activated polypeptide is expressed in layer I and layer III neurons of the VLO of the brain.

16. The method of Aspect 1, wherein the method further comprises reversibly inserting an optical light source in the VLO of the individual.

17. The method of Aspect 1, wherein the method further comprises stimulating the VLO of the brain.

18. The method of Aspect 17, wherein said stimulating the VLO of the brain results in the positive measured fMRI signal at the VLO of the brain.

19. The method of Aspect 1, wherein said stimulating the thalamocortical projections at the light pulse having a frequency ranging from 5 Hz to 20 Hz results in the negative measured fMRI signal in the contralateral region comprising the prefrontal cortex of the brain.

20. The method of Aspect 1, wherein said stimulating the cell bodies at the light pulse having a frequency of 40 Hz or more increases the neuronal activity of the ipsilateral thalamus of the brain.

21. The method of Aspect 1, wherein said stimulating the thalamocortical projections with the light pulse having a frequency ranging from 20-40 Hz activates the neuronal activity of the ipsilateral thalamus of the brain.

22. The method of Aspect 1, wherein said stimulating the cell bodies of the thalamic submedial nucleus results in the positive measured fMRI signal in the ipsilateral thalamus of the brain.

23. The method of Aspect 1, wherein said stimulating the thalamocortical projections with the light pulse having a frequency of 5 Hz or more inhibits the neuronal activity of the ipsilateral thalamus of the brain.

24. The method of Aspect 1, wherein said measuring the fMRI signal comprises measuring a cerebral blood volume (CBV).

25. The method of Aspect 1, wherein the method further comprises administering a second light-activated polypeptide.

26. The method of Aspect 25, wherein the second light-activated polypeptide is administered into a zona incerta (ZI) region of the brain.

27. The method of Aspect 1, wherein the method further comprises performing electrophysiological recordings to detect firing rates of neurons in one or more brain regions associated with the measured fMRI signal.

28. The method of Aspect 27, wherein the one or more brain regions comprises the ipsilateral VLO of the brain.

29. The method of Aspect 28, wherein the positive measured fMRI signal is associated with an increased firing rate of neurons in the ipsilateral VLO.

30. The method of Aspect 27, wherein the one or more brain regions comprises the contralateral VLO.

31. The method of Aspect 30, wherein the negative measured fMRI signal is associated with a decreased firing rate of neurons in the contralateral VLO.

32. The method of Aspect 31, wherein stimulating at the light pulse having a frequency of 10 Hz or more results in a decreased firing rate of neurons in the contralateral VLO.

33. The method of Aspect 27, wherein the one or more brain regions is the ipsilateral motor cortex.

34. The method of Aspect 33, wherein stimulating at the light pulse having a frequency of 10 Hz or more results in a decrease in firing rate of neurons in the ipsilateral motor cortex.

35. The method of Aspect 33, wherein stimulating at the light pulse having a frequency of 40 Hz or more results in an increase in firing rate of neurons in the ipsilateral motor cortex.

36. A method of modulating pain in an individual, the method comprising:

stimulating one or more of thalamocortical projections, thalamic relay neurons, cortical projection neurons, cell bodies in a thalamic submedial nucleus, and cell bodies in the ventrolateral orbitofrontal cortex (VLO) in the brain of the individual with one or more light pulses, wherein neuronal cell bodies in one or more of the VLO and a thalamus of an individual expresses a light-activated polypeptide, and wherein said stimulation modulates pain in an individual.

37. The method of Aspect 36, wherein stimulating one or more of thalamocortical projections, thalamic relay neurons, cortical projection neurons, cell bodies in a thalamic submedial nucleus, and cell bodies in the VLO in the brain with a first set of light pulses inhibits the neuronal activity in response to noxious stimuli.

38. The method of Aspect 36, wherein stimulating one or more of thalamocortical projections, thalamic relay neurons, cortical projection neurons, cell bodies in a thalamic submedial nucleus, and cell bodies in the VLO in the brain with a first set of light pulses inhibits the neuronal activity associated with aversive or painful sensations in the orbitofrontal cortex of the brain.

39. The method of Aspect 36, wherein stimulating one or more of thalamocortical projections, thalamic relay neurons, cortical projection neurons, cell bodies in a thalamic submedial nucleus, and cell bodies in the VLO in the brain with a second set of light pulses activates the neuronal activity associated with aversive or painful sensations in the orbitofrontal cortex of the brain.

40. A system for modulating temporal patterns of neuronal activity in the brain of an individual, the system comprising:

i) a light source configured to stimulate, with a light pulse, one or more of thalamocortical projections, thalamic relay neurons, cortical projection neurons, cell bodies in a thalamic submedial nucleus, and cell bodies in the VLO in the brain of the individual, wherein a light-responsive opsin polypeptide is expressed in cell bodies in one or more of a ventrolateral orbitofrontal cortex (VLO) and a thalamus of the brain; and

ii) a functional magnetic resonance imaging (fMRI) device configured to scan the whole-brain during stimulation to produce an fMRI signal;

wherein a positive measured fMRI signal is associated with an increase in neuronal activity following stimulation, and wherein a negative measured fMRI signal is associated with a decrease in neuronal activity following stimulation.

41. The system of Aspect 40, wherein the whole-brain comprises an ipsilateral and contralateral brain region.

42. The system of Aspect 41, wherein the ipsilateral region comprises a left hemisphere of the brain comprising the a medial prefontal cortex, a lateral prefontal cortex, a motor cortex, a cingulate cortex, a sensory cortex, an insular cortex, a striatum, and a thalamus.

43. The system of Aspect 41, wherein the contralateral region comprises a right hemisphere of the brain comprising a medial prefontal cortex, a lateral prefontal cortex, a motor cortex, a cingulate cortex, a sensory cortex, an insular cortex, a striatum, and a thalamus.

44. The system of Aspect 40, wherein the light pulse has a frequency ranging from 5 Hz to 40 Hz.

45. The system of Aspect 44, wherein the light pulse has a frequency of 10 Hz or more.

46. The system of Aspect 44, wherein the light pulse has a frequency of 40 Hz or more.

47. The system of Aspect 40, wherein stimulation of the thalamocortical projection with the light pulse having a frequency of 10 Hz or more results in the negative measured fMRI signal in the sensory, motor, and cingulate cortex of the ipsilateral region of the brain.

48. The system of Aspect 40, wherein the light source can be reversibly inserted in the VLO of the individual.

49. The system of Aspect 40, wherein stimulation of the thalamocortical projection with the light pulse having a frequency of 40 Hz or more results in the positive measured fMRI signal.

50. The system of Aspect 40, wherein stimulation of the thalamocortical projection with the light pulse having a frequency of 10 Hz or more results in the negative measured fMRI signals in the contralateral region of the brain.

51. The system of Aspect 40, wherein stimulation of the cell bodies in the VLO with the light pulse ranging from 5 Hz to 40 Hz results in the positive measured fMRI signal in the ipsilateral region of the brain.

52. The system of Aspect 40, wherein the light-activated is expressed in layer I and layer III neurons of the VLO of the brain.

53. The system of Aspect 40, wherein the implantable light source is implanted in a dorsal position in the VLO of the brain.

54. The system of Aspect 40, wherein stimulation with the light pulse having a frequency ranging from 5 Hz to 10 Hz inhibits the neuronal activity of the ipsilateral thalamus of the brain.

55. The system of Aspect 40, wherein the fMRI signal comprises a cerebral blood volume (CBV).

56. The system of Aspect 40, wherein the system further comprises a second light-activated polypeptide expressed in neurons in a zona incerta region of the brain.

57. The system of Aspect 40, wherein the system further comprises electrophysiological recording device configured to detect firing rates of neurons in one or more brain regions associated with the measured fMRI signal.

58. The system of Aspect 57, wherein the one or more brain regions comprises the ipsilateral VLO of the brain.

59. The system of Aspect 58, wherein a positive fMRI signal is associated with an increased firing rate of neurons in the ipsilateral VLO of the brain.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Thalamic Input to Orbitofrontal Cortex Drives Brain-Wide, Frequency-Dependent Inhibition Mediated by GABA and Zona Incerta

Optogenetic fMRI was applied to drive various elements of VLO circuitry while visualizing the whole-brain response. Surprisingly, driving excitatory thalamocortical projections to VLO at low frequencies (5-10 Hz) evoked widespread, bilateral decreases in brain activity spanning multiple cortical and subcortical structures. This pattern was unique to thalamocortical projections, with direct stimulations of neither VLO nor thalamus eliciting such a response. High-frequency stimulations (25-40 Hz) of thalamocortical projections evoked dramatically different—though still far-reaching —responses, in the form of widespread ipsilateral activation. Importantly, decreases in brain activity evoked by low-frequency thalamocortical input were mediated by GABA and activity in zona incerta. These findings identify specific circuit mechanisms underlying VLO control of brain-wide neural activities.

Despite evidence that VLO has a global role in brain function, the circuit mechanisms by which it accomplishes such influence have not been studied directly. To better understand how VLO supports different behavioral processes, a technical approach is needed capable of controlling individual circuit elements while visualizing the brain-wide response. Optogenetic fMRI (ofMRI)—the combination of optogenetic stimulation with whole-brain functional magnetic resonance imaging—was applied to directly visualize the global influence of VLO's afferent and efferent connections. Aspects of the present disclosure study how different temporal patterns of activity in the VLO circuit affect brain dynamics by driving its input and output at distinct frequencies.

### Results

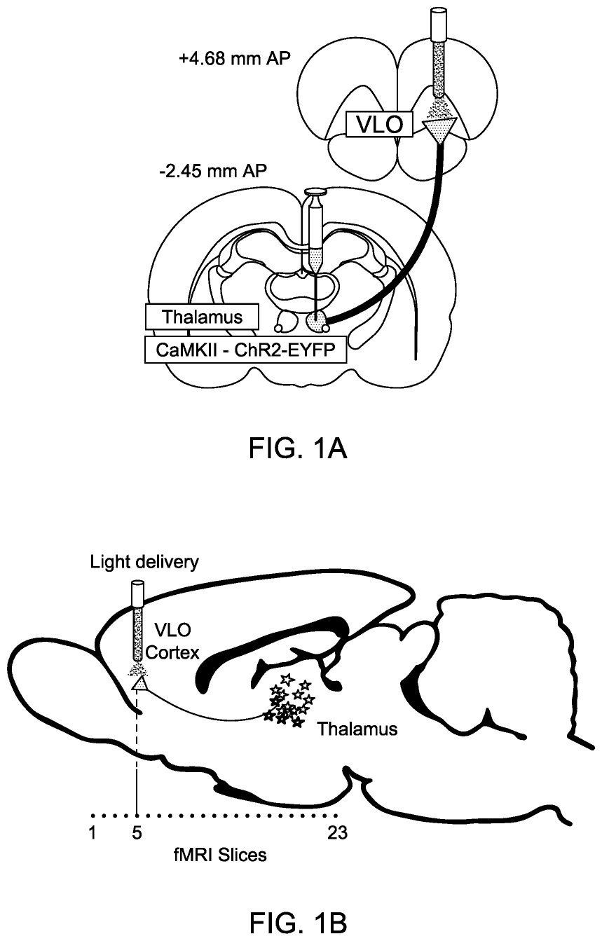

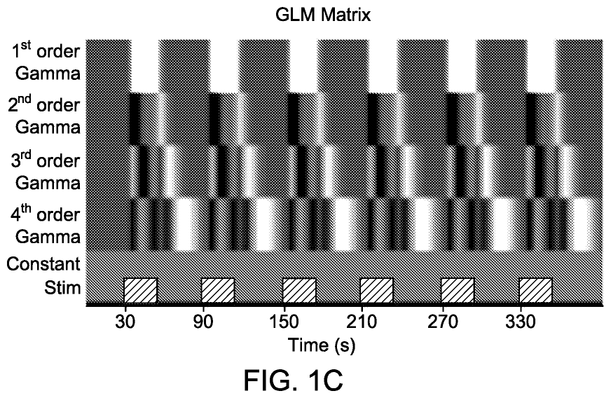

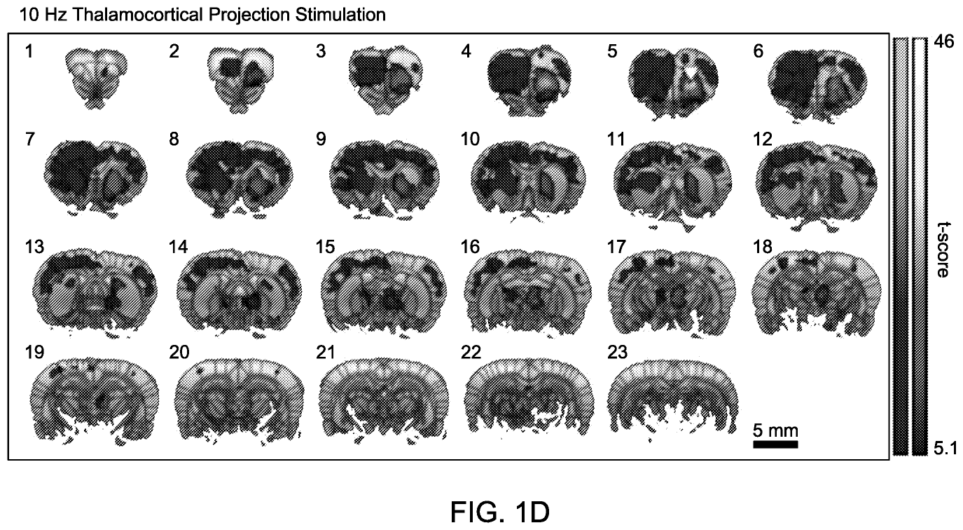

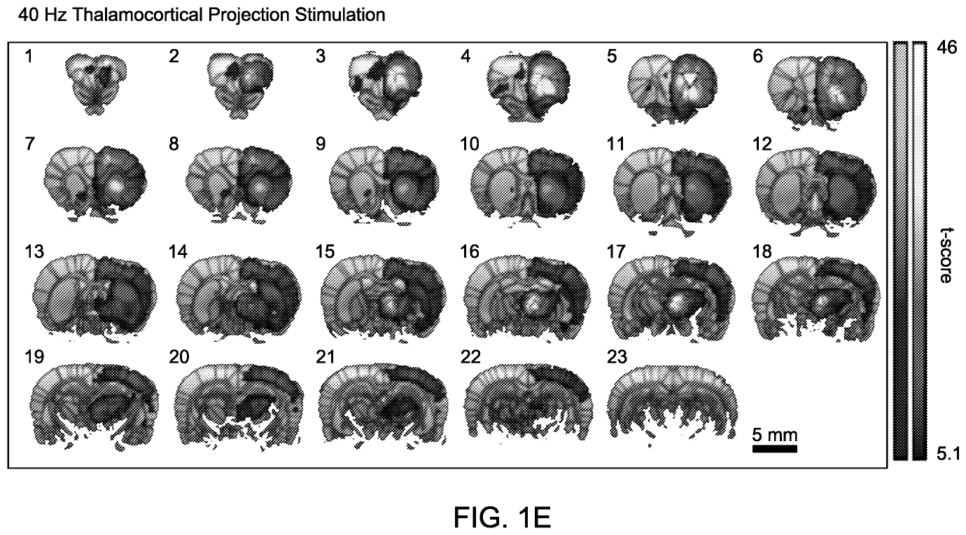

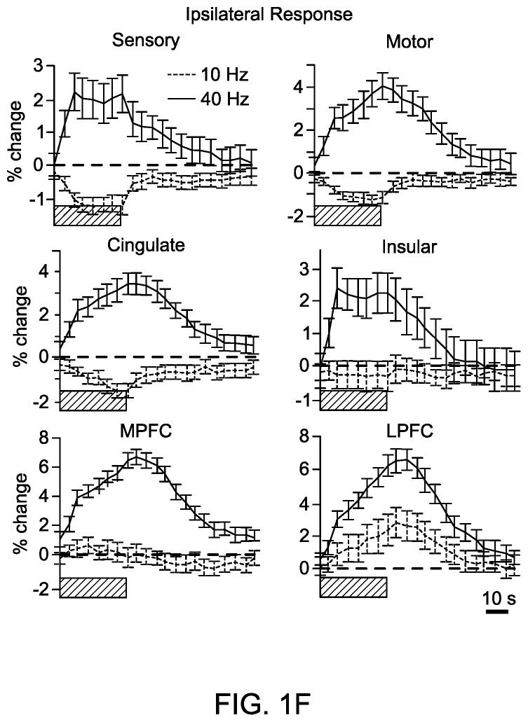

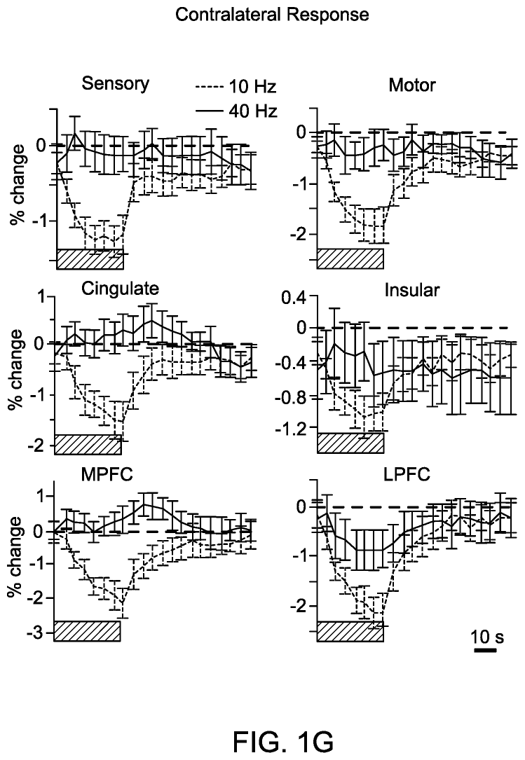

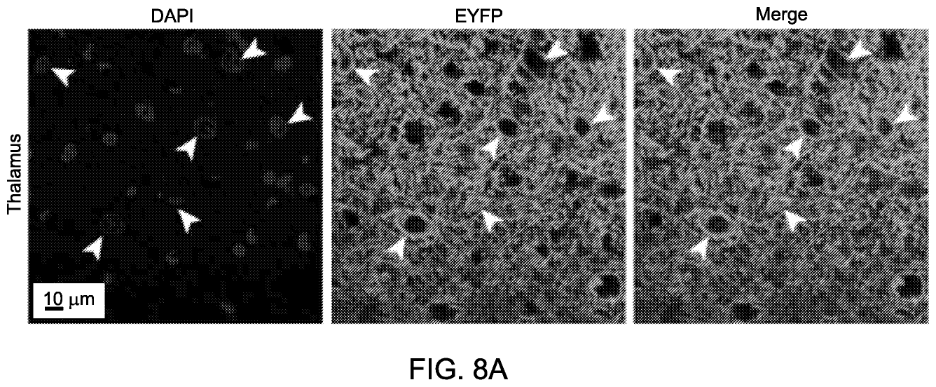

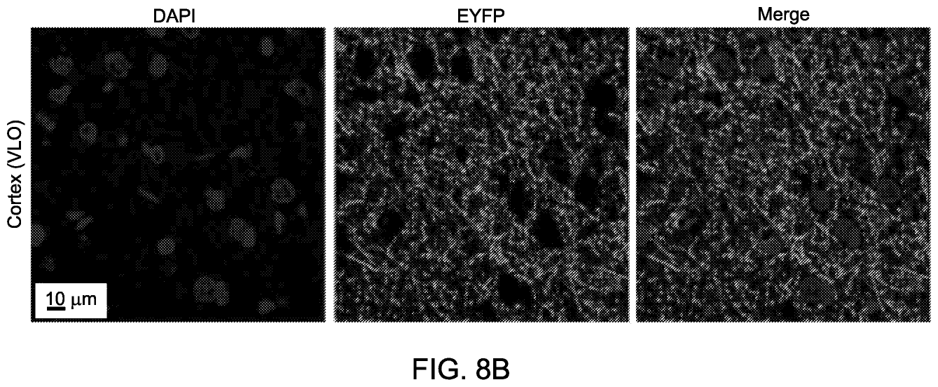

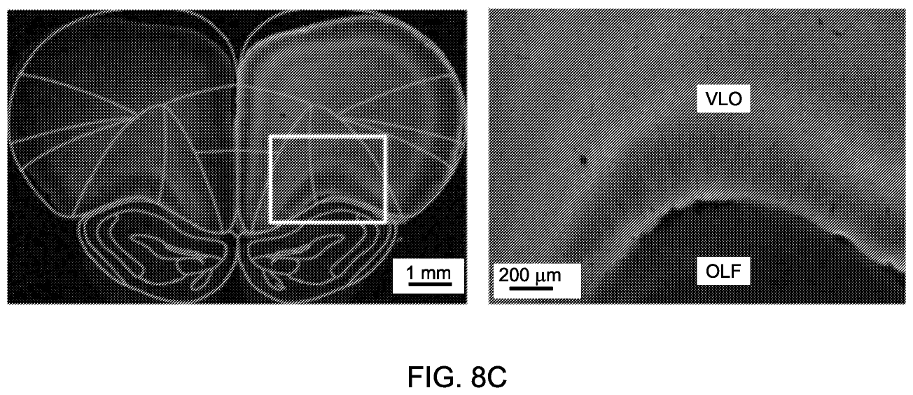

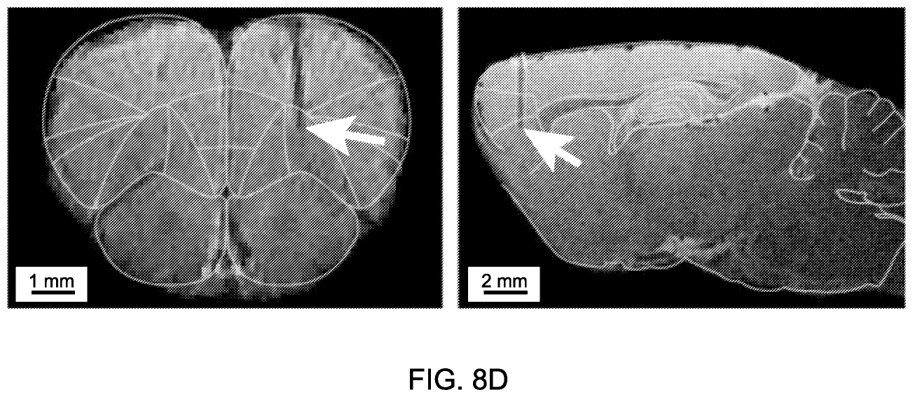

The influence of thalamocortical projections to VLO was first investigated by stimulating thalamic terminals there. Adenoassociated virus carrying the ChR2-EYFP excitatory opsin was injected into the submedial nucleus of thalamus (FIG. 1A). To achieve targeted transfection, the CaMKIIa promoter was used, which in thalamus is primarily expressed in excitatory relay neurons (Smith, 2008). This resulted in strong membrane-bound expression of ChR2 at the site of injection (FIG. 8A). Ex vivo histology confirmed that ChR2 was strongly expressed in layers I and III of VLO (FIGS. 8B-8C), consistent with known termination patterns of the injected nucleus (Krettek and Price, 1977). Stimulation of ChR2-positive terminals in cortex was achieved by implanting an optical fiber in VLO (FIG. 1A).

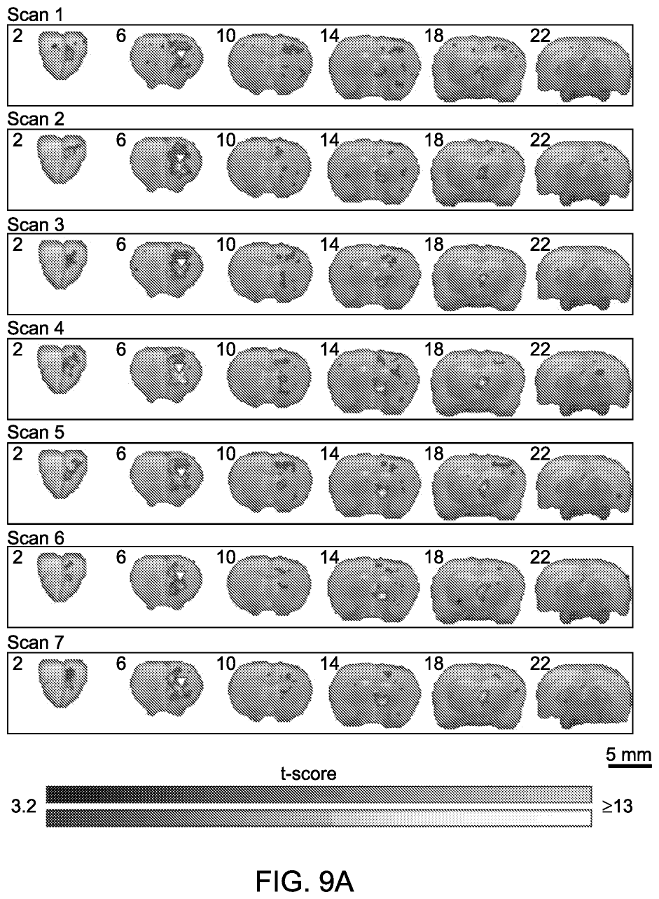

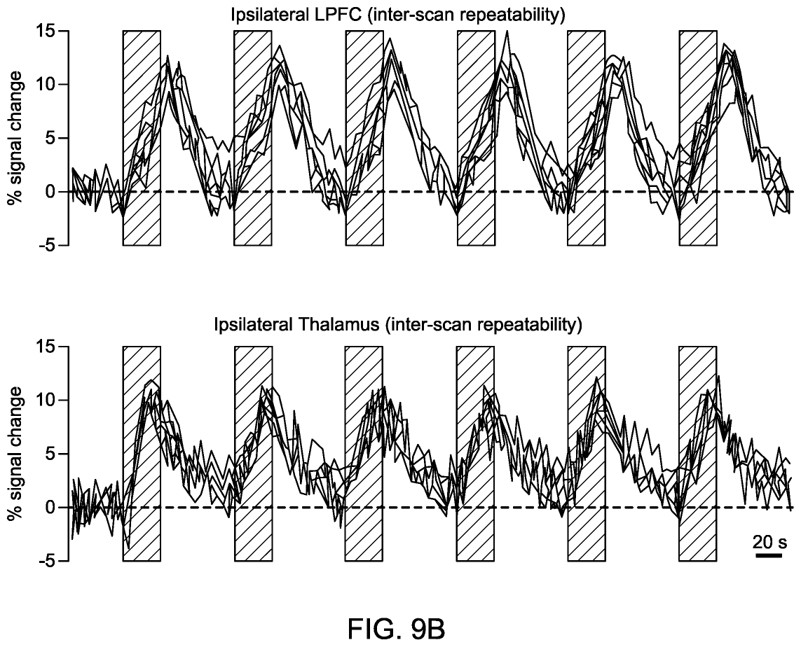

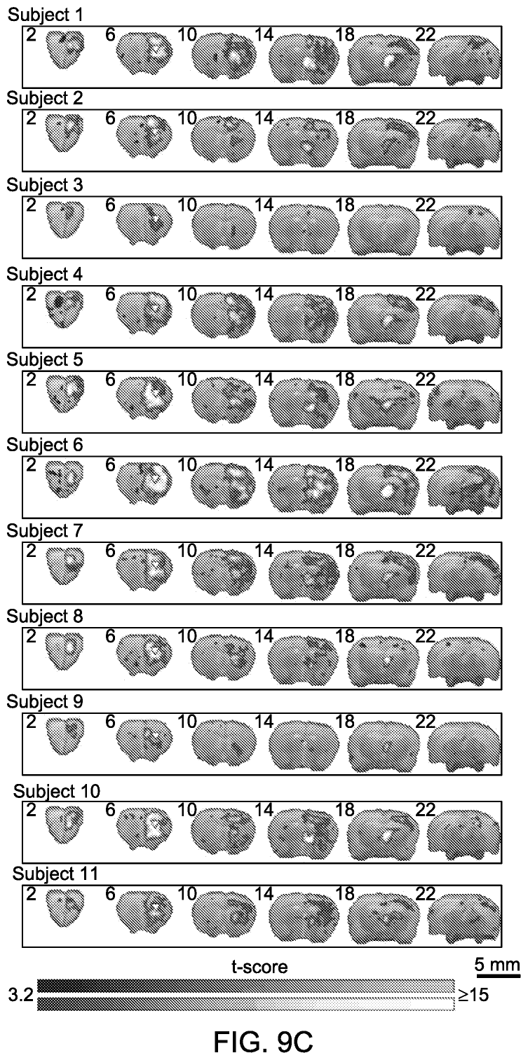

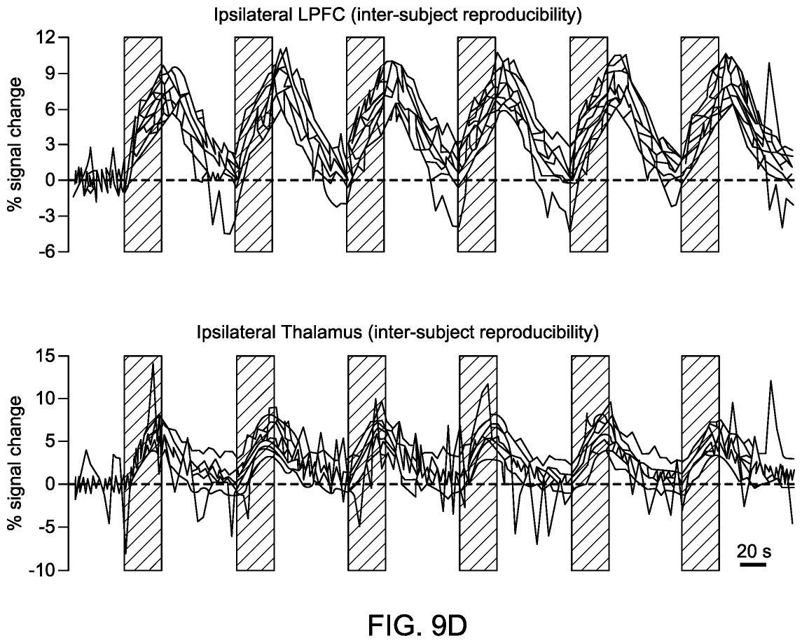

Optogenetic fMRI experiments were conducted in order to visualize the dynamic, brain-wide response to different frequencies of thalamocortical stimulation. Optical pulses were delivered at a frequency of 10 or 40 Hz. Imaging was performed over 23 coronal slices (FIG. 1B). Standard general linear model (GLM) statistical techniques were used to identify voxels significantly modulated during stimulation (FIG. 1C). Responses were highly consistent across scans and subjects (FIGS. 9A-9D).

### Frequency of Thalamocortical Stimulation in VLO Controls Interhemispheric Modulation

fMRI activation maps show that stimulation frequency was a critical parameter in determining the spatial extent of ipsilateral and contralateral modulation (FIGS. 1D,1E). Both stimulation frequencies resulted in a robust positive response at the site of stimulation in VLO, as well as in the ipsilateral thalamus and striatum. 10 Hz stimulation drove a bilateral negative measured response that spanned across cortex, contralateral striatum, and contralateral thalamus. 40 Hz stimulation caused robust positive activations throughout ipsilateral cortex, but the contralateral hemisphere was mostly devoid of any modulation. Only a marginal negative response in prefrontal cortex and striatum was observed.

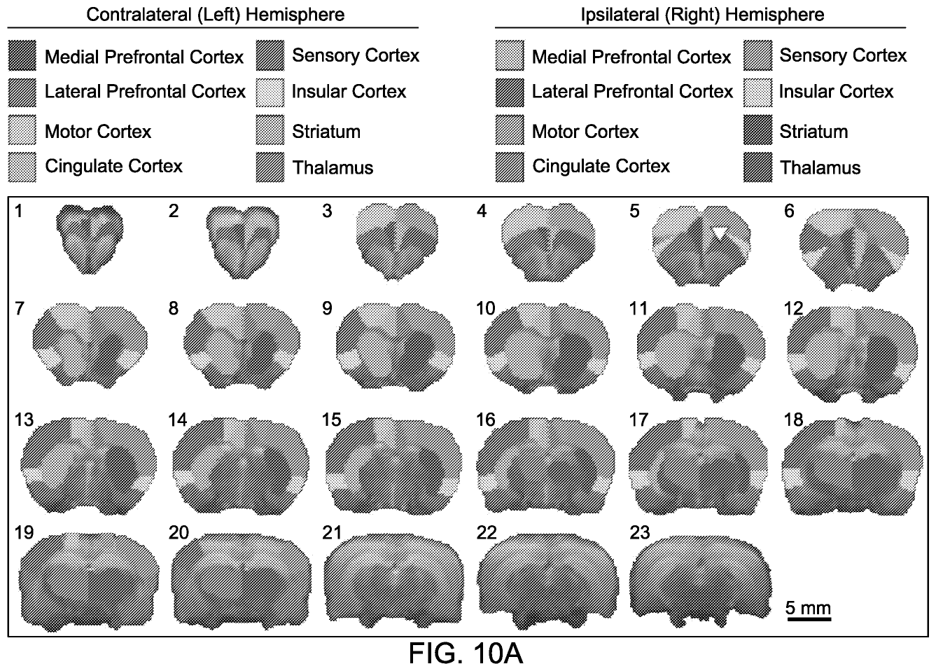

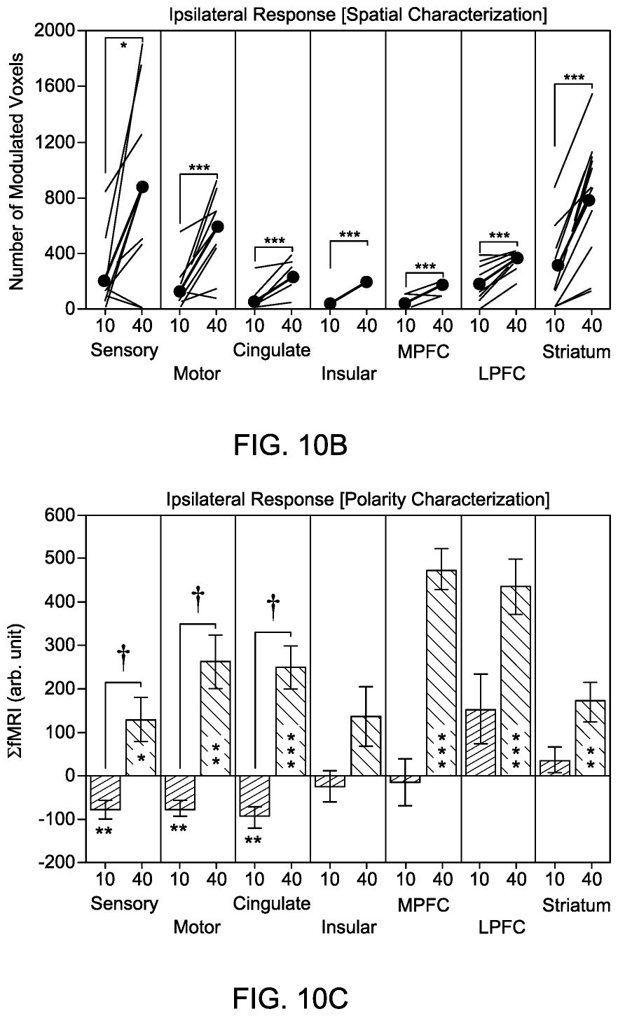

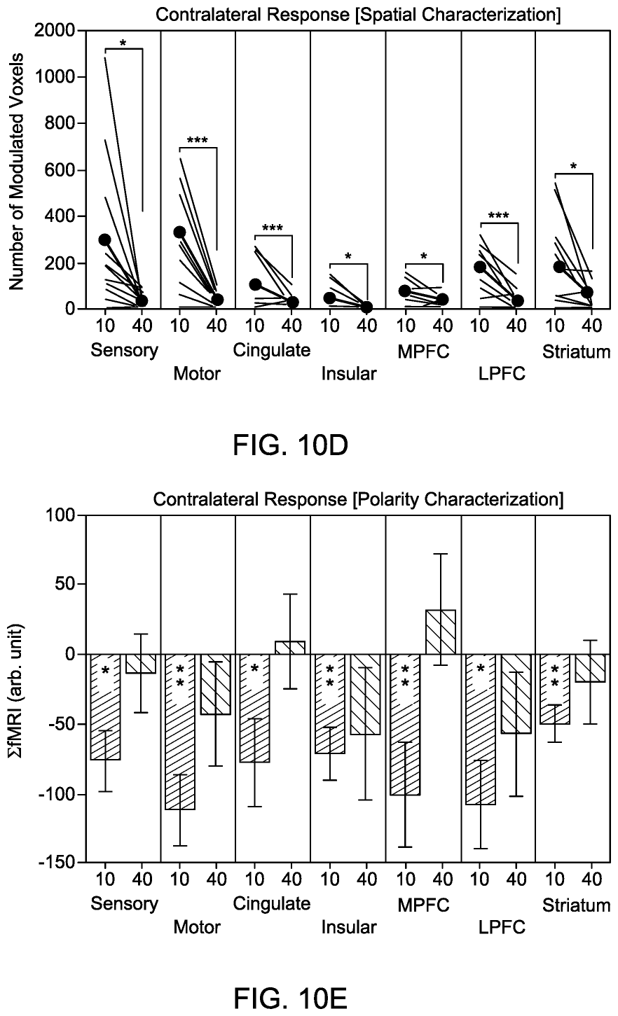

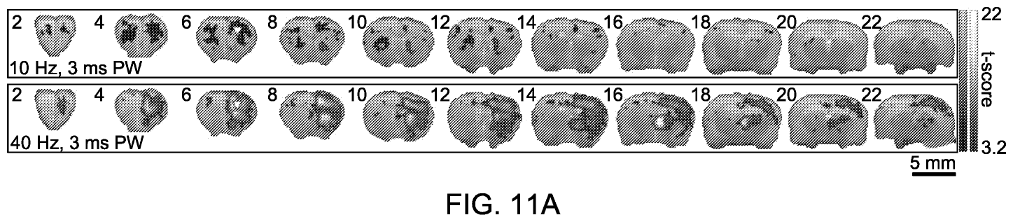

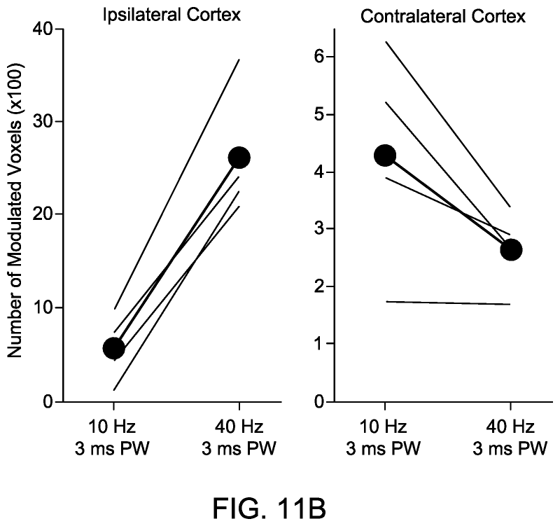

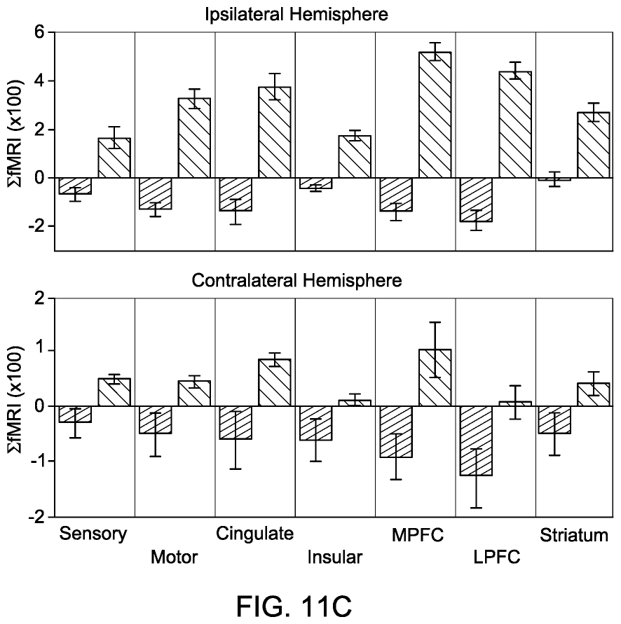

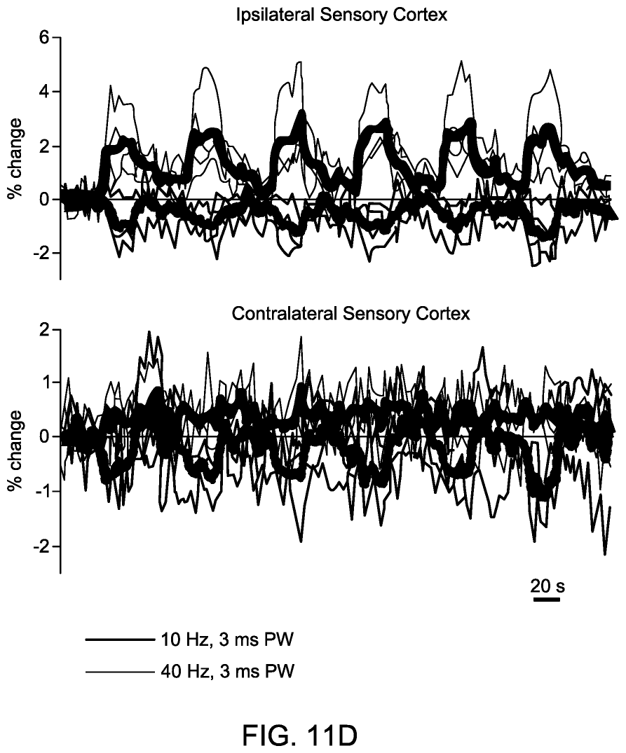

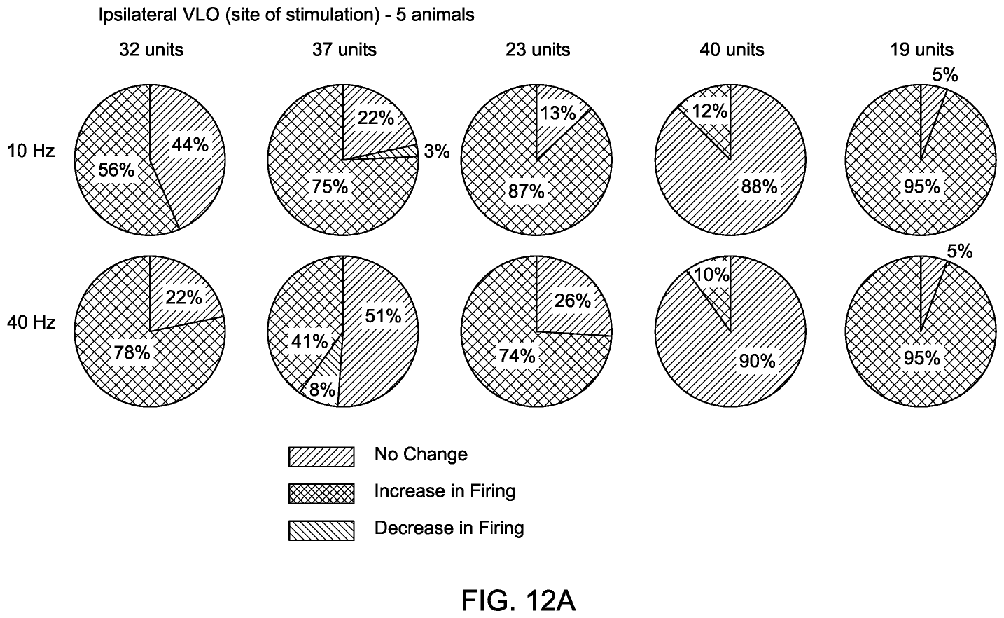

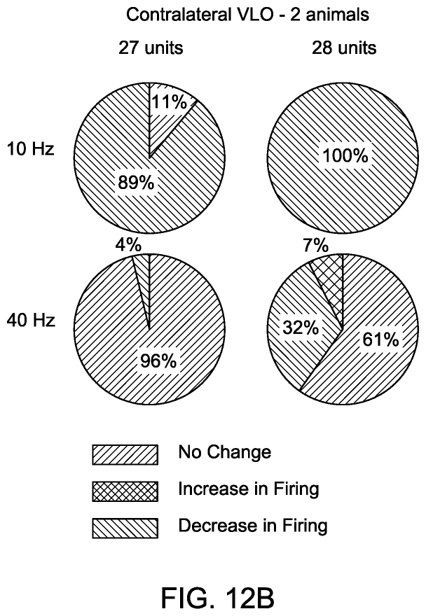

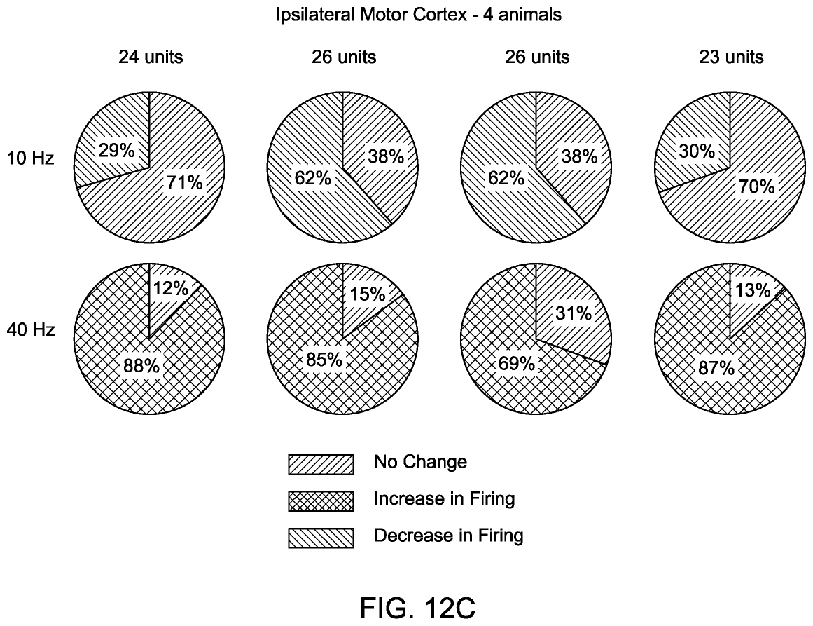

To quantify response patterns, the number of significantly modulated voxels was calculated in anatomically defined regions of interest (ROIs; FIG. 10A). In the ipsilateral hemisphere, the number of modulated voxels increased between 10 and 40 Hz for all segmented areas of cortex and striatum (FIG. 10B; p<0.05, N=11 animals). Conversely, in the contralateral hemisphere, the amount of modulated volume decreased between 10 and 40 Hz (FIG. 10D). These results indicate that the firing rate of thalamic input to VLO determines the spatial extent of downstream modulation. The ipsilateral hemisphere is modulated most during 40 Hz stimulation, while the contralateral hemisphere is modulated most during 10 Hz stimulation. The same frequency-dependent trends were observed when a constant pulse width was used in control experiments (FIGS. 12A-12B).

### Frequency of Thalamocortical Stimulation in VLO Controls the Polarity of Evoked Responses

The temporal dynamics of brain-wide responses was examined next. Quantitative measurements of each ROI's response polarity were calculated as the sum of its average fMRI time series (YfMRI). In the ipsilateral hemisphere, sensory, cingulate, and motor cortex exhibited a significant negative response during 10 Hz stimulation (FIG. 10C; p<0.05, N=11 animals). These same regions, with striatum, LPFC, and MPFC, exhibited a significant positive response during 40 Hz stimulation. Visualizing the time series throughout ipsilateral cortex largely corroborated YfMRI measurements (FIG. 1F). Sensory, cingulate, and motor cortex all displayed a robust negative response during 10 Hz stimulation that switched to positive during 40 Hz stimulation.

Quantitative measurements of YfMRI in the contralateral hemisphere varied greatly from those observed in the ipsilateral hemisphere. The cortex and striatum exhibited significant negative responses during 10 Hz stimulation (FIG. 10E; p<0.05, N=11 animals), but no YfMRI values were significantly different from zero during 40 Hz stimulation. Visualizing the time series throughout contralateral cortex confirmed that activity there sharply decreased during 10 Hz stimulation (FIG. 1G). fMRI responses to 40 Hz stimulation were generally flat or—in the case of LPFC—displayed a minor negative deflection. These data illustrate the widespread, bilateral influence that thalamic inputs to VLO exert by suppressing remote activity in a frequency-dependent manner. Importantly, this effect was preserved when pulse width was held constant in control experiments (FIGS. 12C-12D), confirming that stimulation frequency was the primary factor in determining the polarity of stimulus-evoked responses.

### Frequency Sweep Experiments Reveal Region-Dependent Transitions in Response Patterns

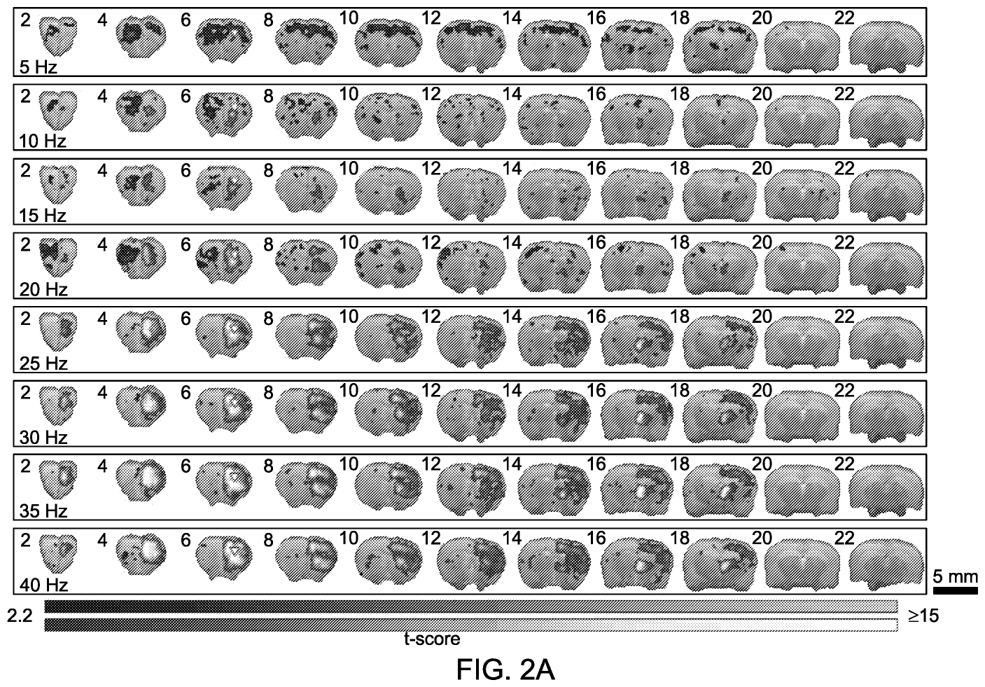

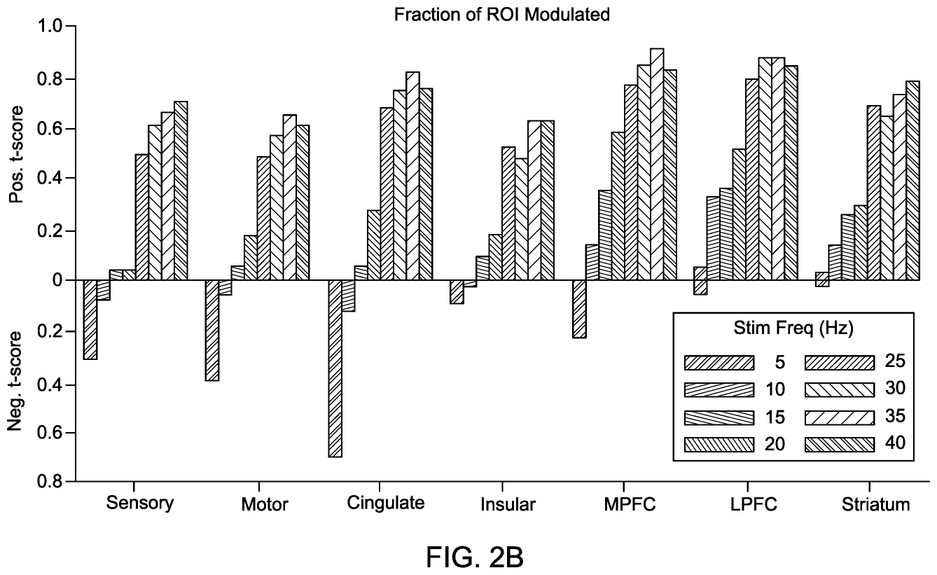

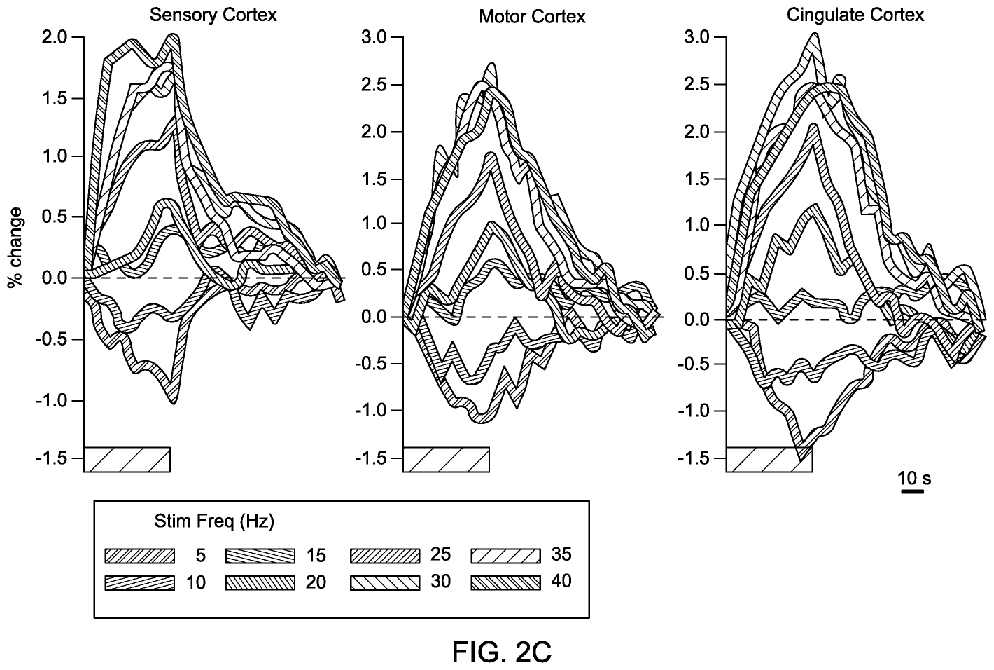

In order to explore how frequency-dependent changes manifest, a second series of imaging sessions was conducted with a subset of animals reported above (N=7). Stimulations were performed over frequencies ranging from 5 to 40 Hz in 5 Hz intervals. The resulting activation maps are shown in FIG. 2A. Stimulation at all frequencies evoked a positive response at the site of stimulation. The negative response observed throughout the contralateral hemisphere during 10 Hz stimulation was observed from 5 Hz to frequencies as high as 20 Hz. At 25 Hz and above, negative responses in the contralateral hemisphere were generally limited to prefrontal cortex. Interestingly, it was also at 25 Hz that widespread activation of the ipsilateral cortex started to manifest. To quantify this effect, the percentage of voxels were examined that were significantly modulated at each frequency (FIG. 2B). Several regions exhibited a large increase in positive modulation volume between 20 and 25 Hz, suggesting that a threshold for widespread forebrain activation had been reached. Negative modulation volumes were also greater during 5 Hz stimulation compared to 10 Hz stimulation, suggesting that the circuit mechanism responsible for negative signals has an even stronger effect at lower frequencies. Notably, the transition from negative to positive responses observed in sensory, motor, and cingulate cortex occurred between 10 and 15 Hz stimulation. Time series extracted from these ROIs confirmed this trend (FIG. 2C).

Thalamocortical Projections to VLO Uniquely Drive Widespread Negative fMRI Signals

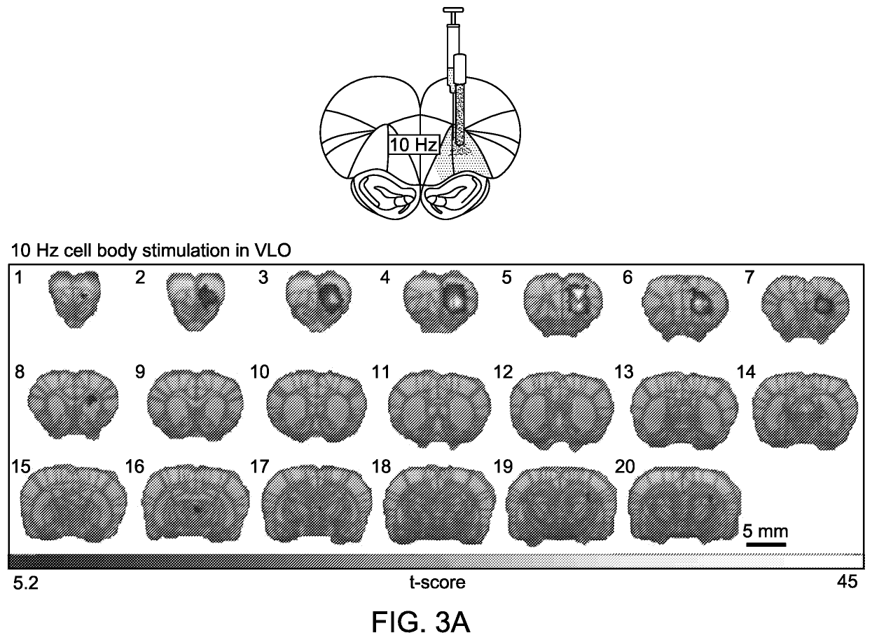

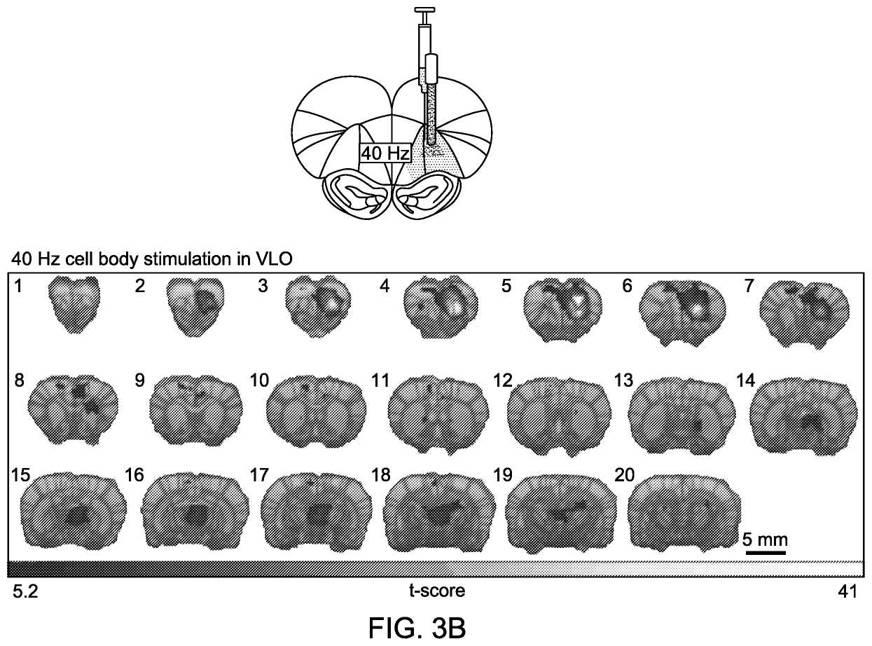

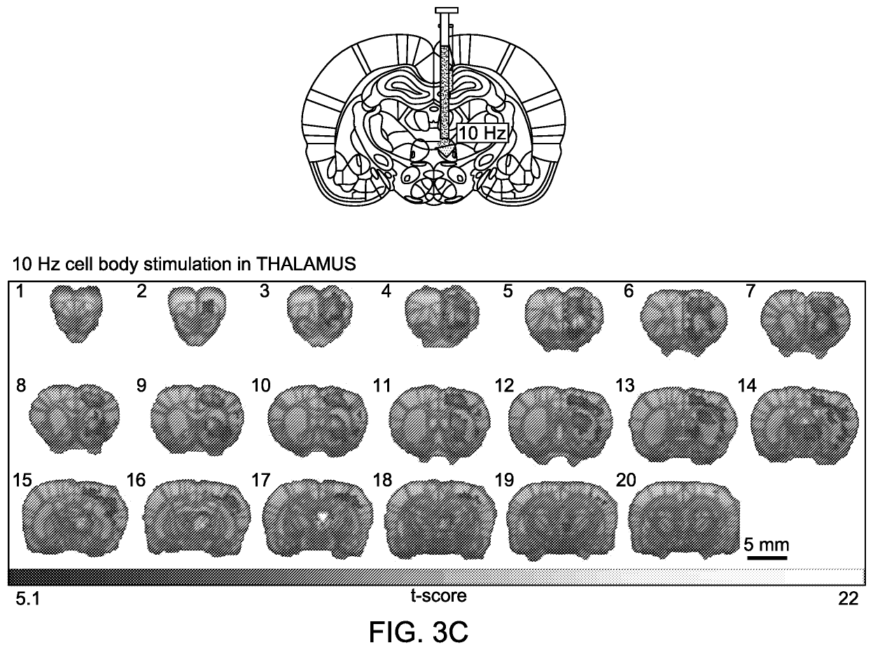

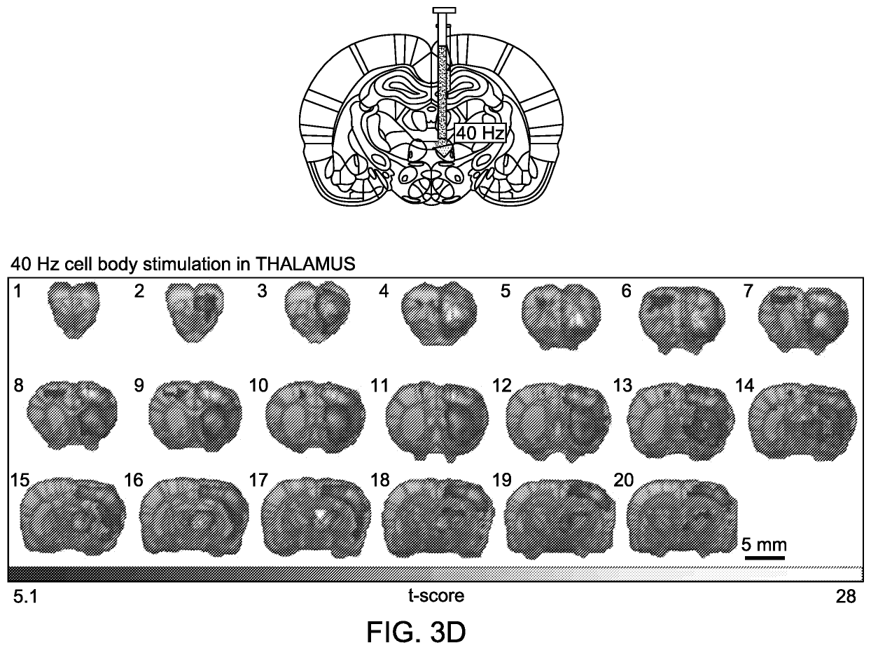

The thalamocortical projections to VLO represent only one neuronal element in the perturbed circuit. To better understand the source of fMRI responses, pyramidal neurons were also stimulated in VLO. Neither 10 nor 40 Hz stimulation of cell bodies in VLO drove a negative fMRI response in any region (FIGS. 3A-3B). Similar to stimulation of thalamocortical projections, exciting cell bodies at 40 Hz drove activation of the ipsilateral thalamus. However, the widespread cortical activations observed during stimulation of thalamocortical projections did not occur. These data suggest that direct activation of VLO does not cause the same frequency-dependent or widespread inhibitory effects induced by thalamic input to this region. Cell bodies in the thalamic submedial nucleus were then stimulated, which projects heavily to VLO. As shown in the group-level activation maps (FIGS. 3C-3D), driving these relay neurons at 10 and 40 Hz elicits a strong response in VLO, but fails to evoke a negative fMRI response in any region. Thus, directly stimulating the projections to VLO elicits a fundamentally different response than stimulating cell bodies that project there.

Neuronal Underpinnings of Brain-Wide, Frequency-Dependent fMRI Signals

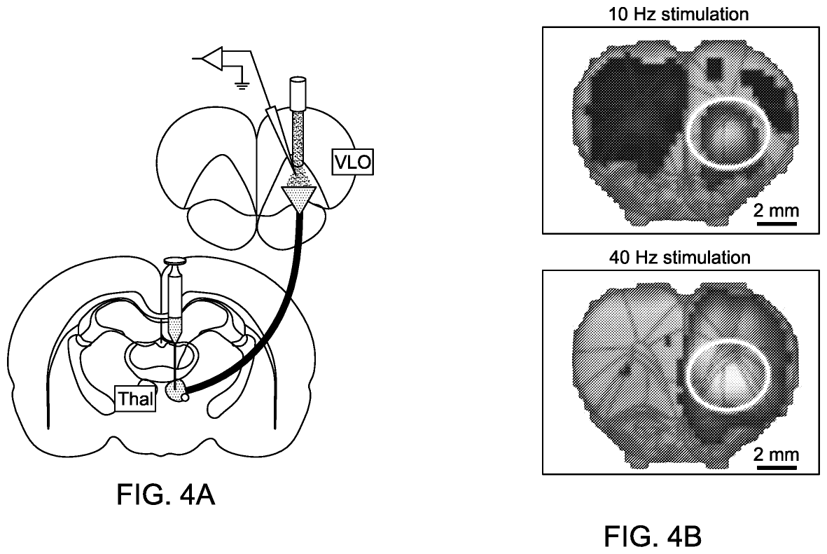

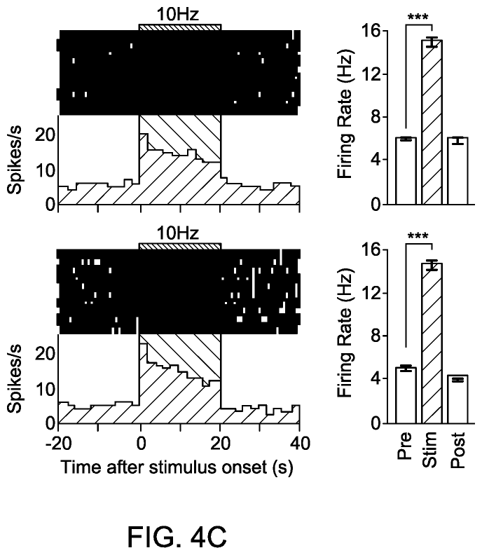

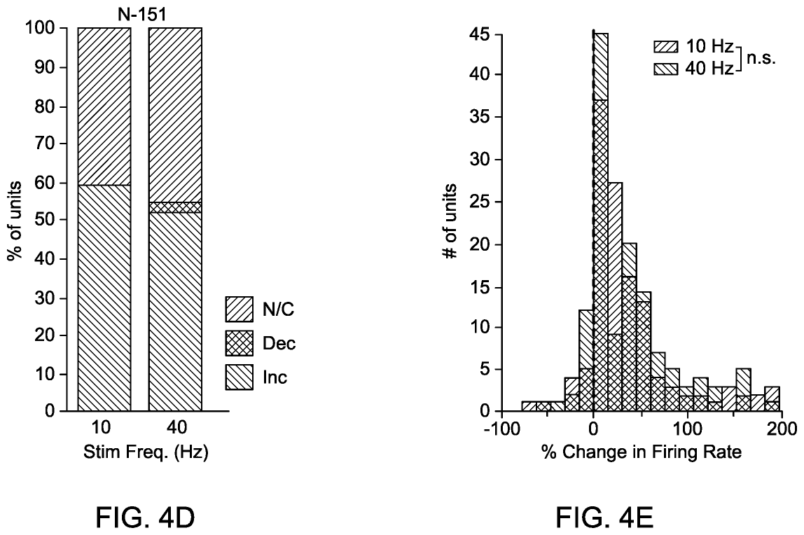

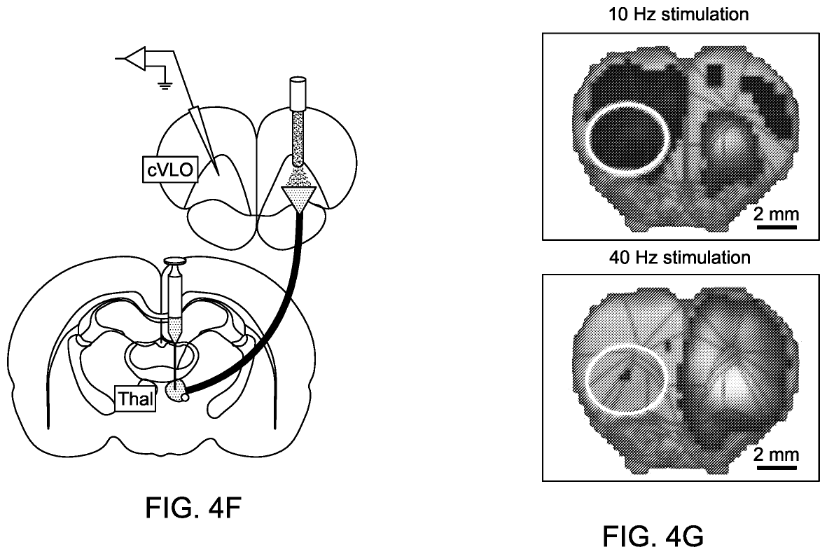

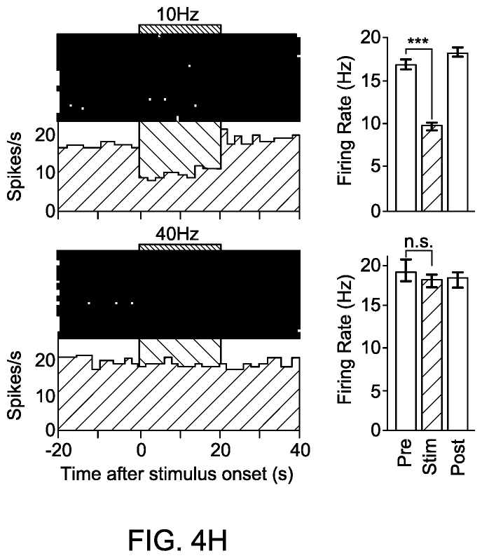

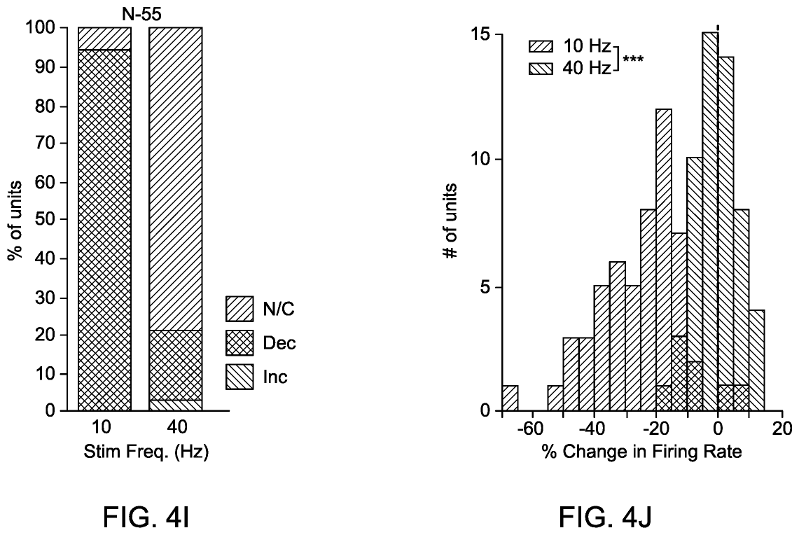

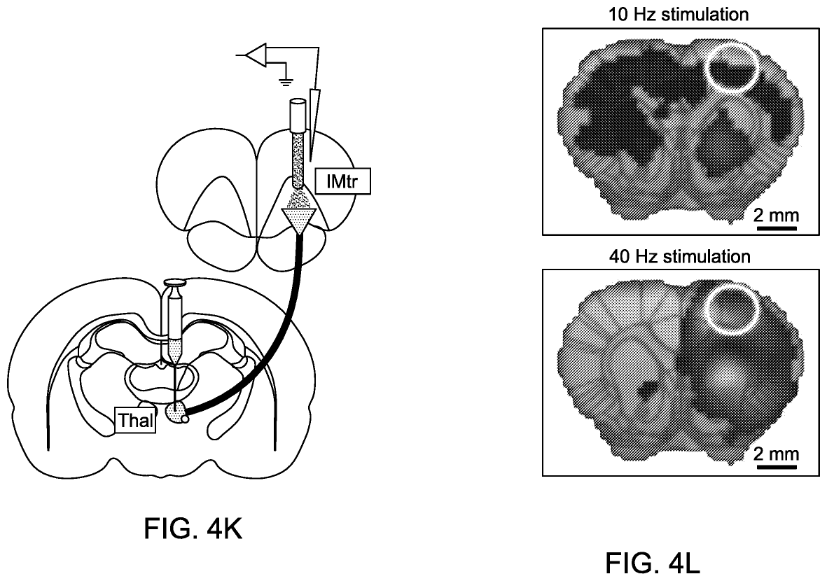

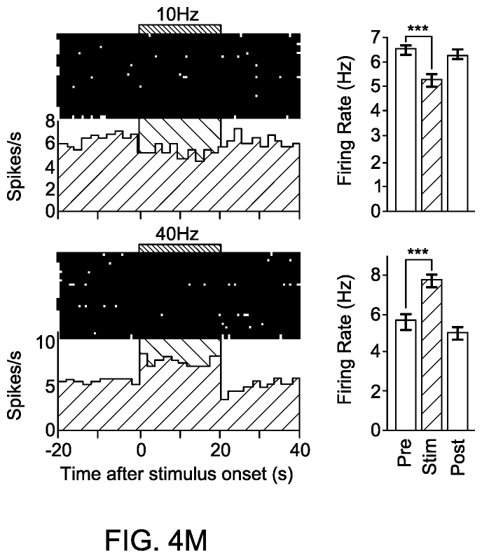

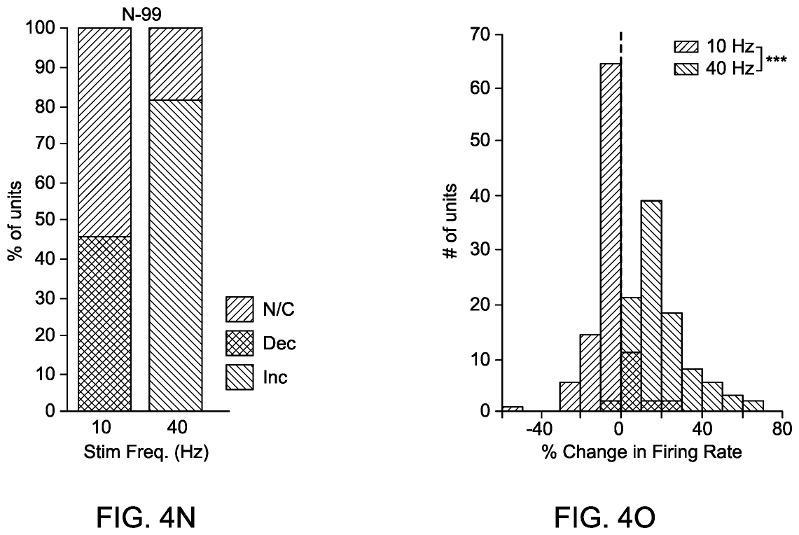

Across the brain, several classes of frequency-dependent fMRI responses were observed during stimulation of thalamic input to VLO (FIGS. 1A-1G). To investigate how these dynamics relate to neuronal activity, a series of in vivo electrophysiology experiments were performed. Extracellular recordings at the site of stimulation in ipsilateral VLO were first performed (FIG. 4A), where the fMRI response was positive during stimulation at both 10 and 40 Hz (FIG. 4B). Peri-event time histograms from a representative unit show that these signal changes were associated with corresponding increases in spiking (FIG. 4C). Across all recorded units, over half were modulated by stimulation at either frequency (FIG. 4D and FIG. 12A; 60% and 54% during 10 and 40 Hz stimulation, respectively; N=151 units, 5 animals, 10 trials per frequency). Furthermore, nearly all modulated units exhibited a significant increase in firing rate (99% and 96%, respectively). The median change in firing rate was not significantly different between the two frequencies (FIG. 4E). These results confirm that positive fMRI signals observed at the site of stimulation reflect underlying increases in neuronal activity.

It was next investigated whether the negative fMRI signals observed throughout contralateral cortex during 10 Hz stimulation reflect underlying decreases in neuronal activity, and whether this modulation is suppressed at higher stimulation frequencies as suggested by fMRI. Extracellular recordings were performed in contralateral VLO (cVLO; FIG. 4F), where a negative fMRI signal was observed during 10 Hz stimulation, but little modulation was observed during 40 Hz stimulation (FIG. 4G). Peri-event time histograms from a representative unit in cVLO confirm that this pattern was observed at the level of single unit activity (FIG. 4H). Over all recorded units, 95% exhibited a significant decrease in firing rate during 10 Hz stimulation (FIG. 4I and FIG. 12B; N=55 units, 2 animals, 20 trials per frequency). During 40 Hz stimulation, 78% of recorded units exhibited no significant change, and only 18% exhibited a significant decrease in activity. The median change in firing rate was significantly different between the two frequencies (FIG. 4J). These results confirm that low frequencies of thalamic input to VLO preferentially drive decreases in neuronal spiking in the contralateral cortex.

Finally, it was examined whether the frequency-dependent switch in fMRI polarity observed in ipsilateral cortex was associated with corresponding changes in neuronal activity. Recordings in the ipsilateral motor cortex (FIG. 4K) were then performed, where a negative fMRI signal was observed during 10 Hz stimulation, but a positive signal was evoked during 40 Hz stimulation (FIG. 4L). Peri-event time histograms from a representative unit show that this behavior was consistent with underlying spiking dynamics (FIG. 4M). Across all recorded units, 46% exhibited a significant decrease in firing rate during 10 Hz stimulation (FIG. 4N and FIG. 12C; N=99 units, 4 animals, 20 trials per frequency) with the remaining units showing no significant change. During 40 Hz stimulation, 82% of recorded units exhibited a significant increase in firing rate and none exhibited a significant decrease. The median change in firing rate was significantly different between the two frequencies (FIG. 4O). These data confirm that the frequency-dependent switch in cortical response polarity measured with fMRI reflects underlying spiking activity.

### Mechanisms of Evoked Decreases in Cortical Activity

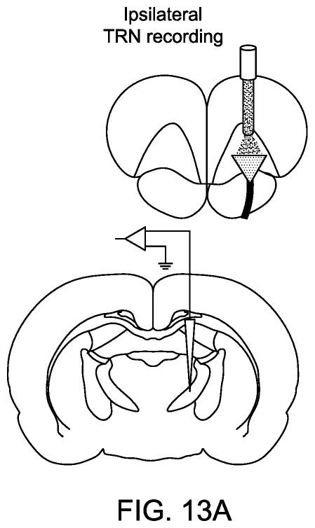

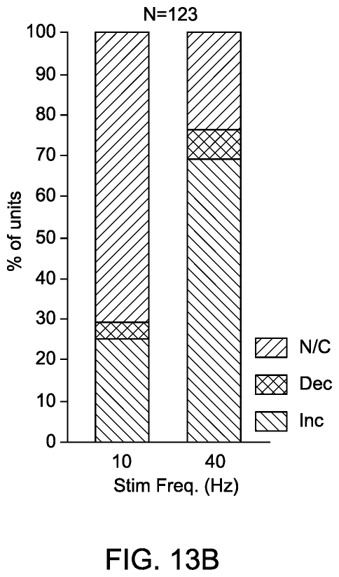

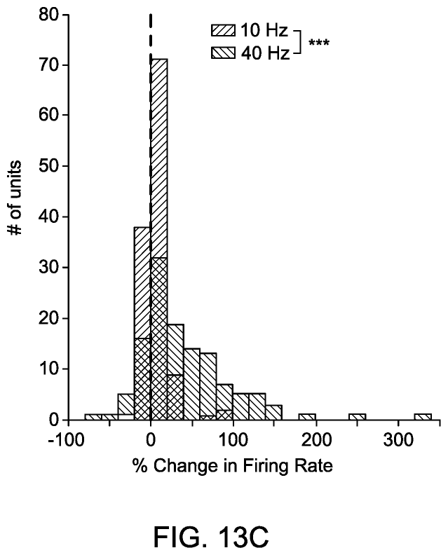

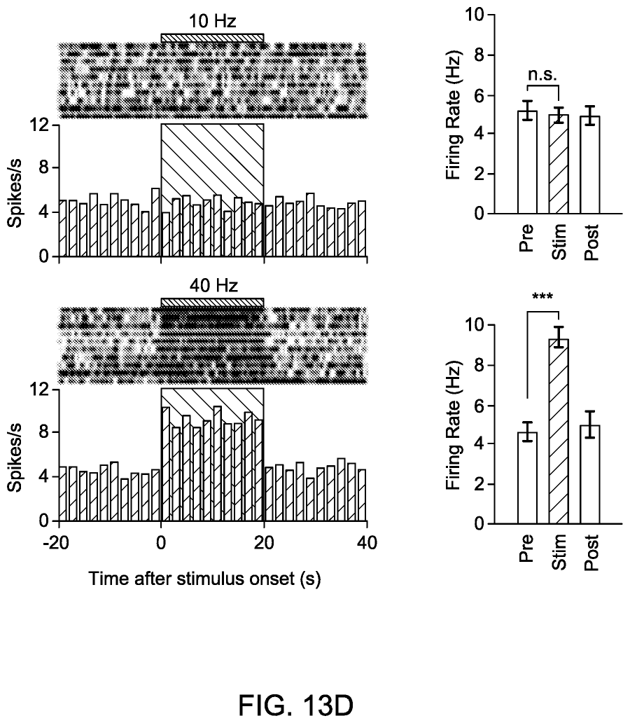

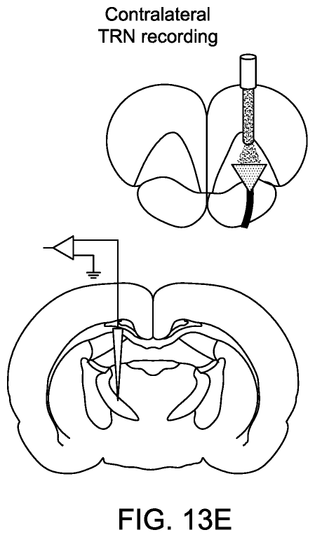

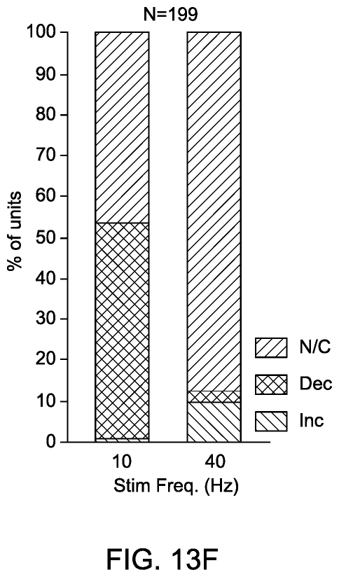

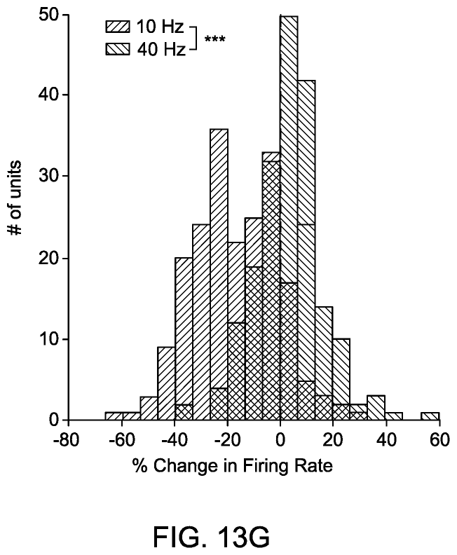

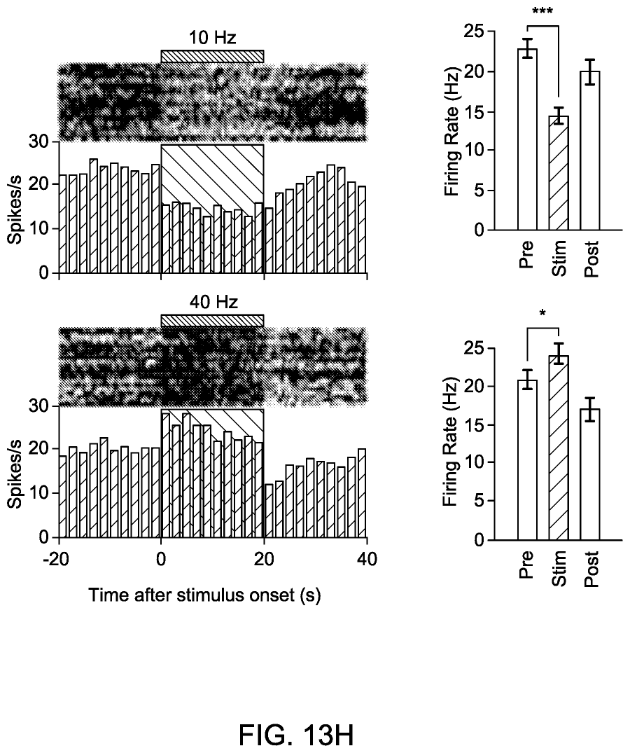

Having established that negative fMRI signals in cortex reflect a decrease in neuronal spiking, the next step was to identify the mechanism for this frequency-dependent response. It was hypothesized that the thalamic reticular nucleus (TRN) might play a role, since it directly inhibits thalamic nuclei and can suppress excitatory input to cortex (Lewis et al., 2015, eLife 4, e08760; Pinault, 2004, Brain Res Rev 46, 1-3). To examine whether activity in TRN was correlated with decreases in cortical firing, extracellular recordings were performed there during 10 and 40 Hz thalamocortical stimulation (FIG. 13A). Given the frequency-dependent nature of evoked decreases in cortical activity, it was predicted that 10 Hz stimulation would evoke the strongest response in TRN. However, the percentage of units exhibiting a significant increase in firing rate more than doubled between 10 and 40 Hz stimulation (FIG. 13B; 25% and 69%, respectively; N=123 units, 2 animals, 10-20 trials per frequency). The median change in firing rate across recorded units was significantly different between the two frequencies, with 40 Hz stimulation driving greater changes (FIGS. 13C-13D). To investigate whether bilateral pathways were involved, recordings in the contralateral reticular nucleus were performed (FIG. 13E). During 10 Hz stimulation, 54% of units there were modulated, of which 98% exhibited a significant decrease in firing rate (FIG. 13F, N=199 recorded units, 20 trials per frequency). During 40 Hz stimulation, 13% of units were modulated, of which 80% exhibited a significant increase in firing rate. Over all recorded units, the median change in firing rate was significantly different between the two frequencies, with 40 Hz stimulation again driving greater changes (FIGS. 13G-13H). These data suggest that inhibition from TRN onto thalamus is not a major cause of the decreases in cortical activity observed during low-frequency thalamocortical stimulation.

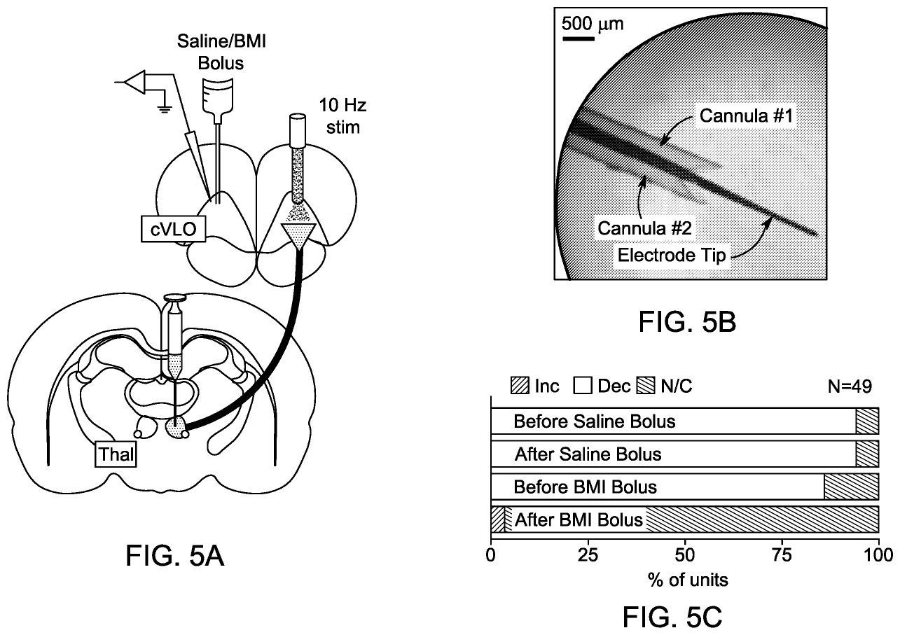

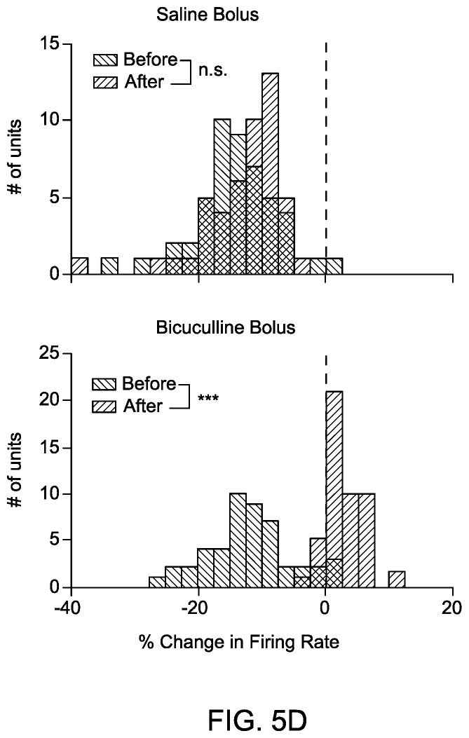

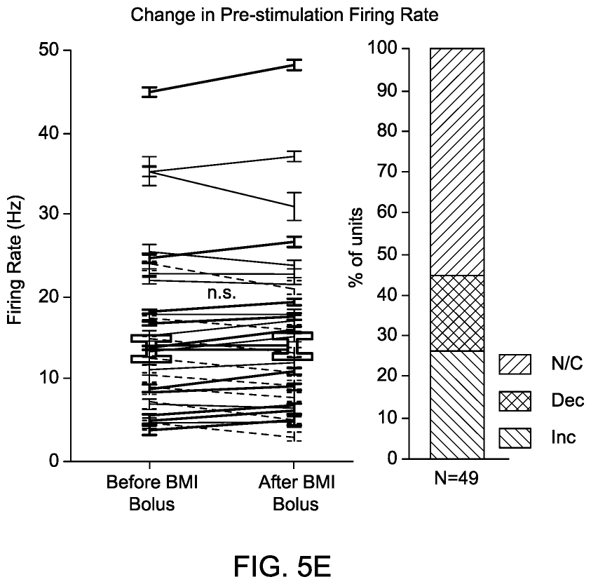

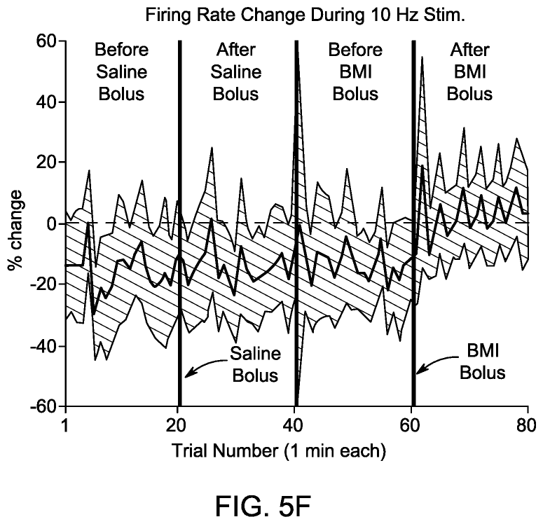

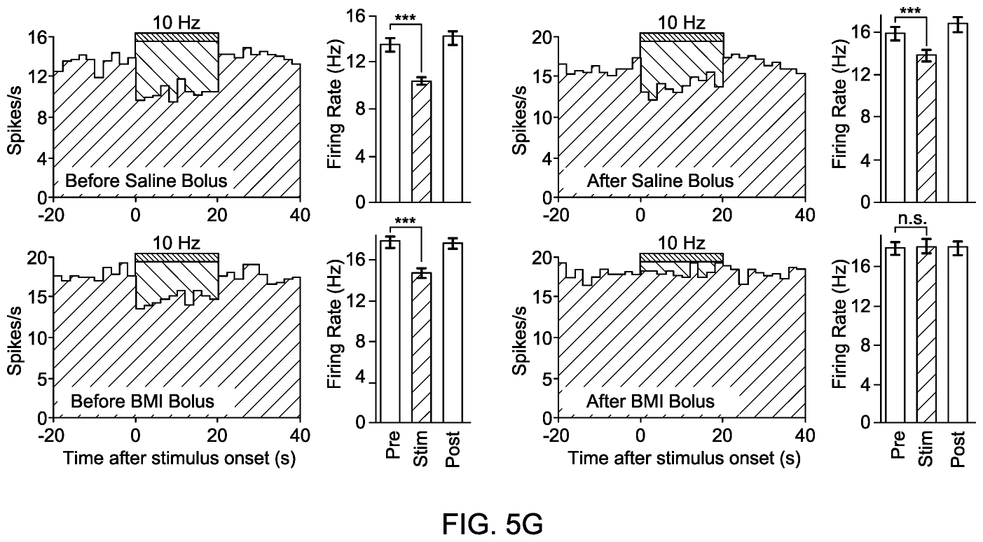

It was next hypothesized that decreases in cortical activity reflect direct GABA-mediated inhibition. The dependence of this response on downstream GABA release was tested by comparing evoked changes in firing rate before and after micro-infusion of bicuculline methiodide (BMI). Although BMI can exhibit mixed pharmacological effects (e.g. blockage of calcium-activated potassium channels), it is a strong antagonist of GABA_{A} receptors. Single-unit recordings were performed in cVLO, where negative fMRI signals were observed during 10 Hz thalamocortical stimulation (FIG. 5A). To ensure that any changes associated with BMI infusion were due to its pharmacological effect, infusions with sterile saline were first performed. To inject both BMI and saline, a pair of cannulas was attached to the recording electrode directly above the electrical contacts (FIG. 5B).

In line with expectations, saline infusion had a negligible effect on cortical inhibition evoked by 10 Hz stimulation. Among 49 units recorded in cVLO, 94% exhibited a significant decrease in firing rate during 10 Hz stimulation both before and after saline infusion (FIG. 5C; N=2 animals, 20 trials per condition). Furthermore, saline infusion did not have a significant effect on the median change in firing rate evoked by 10 Hz stimulation (FIG. 5D). In contrast, infusion of BMI completely eliminated the stimulus-evoked inhibition (FIG. 5C). Prior to BMI infusion, 10 Hz stimulation evoked a significant decrease in firing rate in 86% of units. After BMI infusion, the recorded units were either not modulated by stimulation (96%) or exhibited a significant increase in firing rate (4%). The median change in firing rate evoked by stimulation was also significantly different (FIG. 5D). Importantly, the baseline firing rate in cVLO did not change following BMI infusion in a majority of units (FIG. 5E). Those that did exhibit a difference were roughly split between increases (27%) and decreases (18%). Furthermore, across all units, the average baseline firing rate was not significantly different after BMI infusion (p=0.66). These analyses suggest the drug's effect was related to stimulus-evoked GABA release and not a general decrease in local inhibitory action. The reduction in stimulus-evoked inhibition took immediate effect after BMI infusion and continued throughout the following twenty trials (FIG. 5F). Peri-event time histograms from a representative unit illustrate how stimulus-evoked decreases in firing rate were observed after saline infusion but eliminated after BMI infusion (FIG. 5G). These findings suggest that the widespread cortical inhibition driven by low-frequency thalamic input to VLO is mediated by the release of GABA at remote sites downstream from excited terminals.

Cortical inhibition driven by thalamic input to VLO is mediated by zona incerta

Having established that GABA drove decreases in cortical activity, potential sources of the inhibitory neurotransmitter were identified and the role of zona incerta (ZI) was investigated. In addition to receiving collaterals of the stimulated projection and sparse input from VLO (Kuramoto et al., 2017, The Journal of comparative neurology 525, 3821-3839; Shammah-Lagnado et al., 1985, Neuroscience 15, 109-134), the ZI sends bilateral GABAergic projections throughout neocortex (Lin et al., 1990, Science 248, 1553-1556) and has previously been shown to mediate cortical decreases in firing rate resulting from 10 Hz thalamic stimulation (Liu et al., 2015, eLife 4, e09215). To examine whether this region mediated the widespread inhibition observed throughout forebrain, its activity was inactivated during simultaneous 10 Hz thalamocortical stimulations and recordings in cVLO.

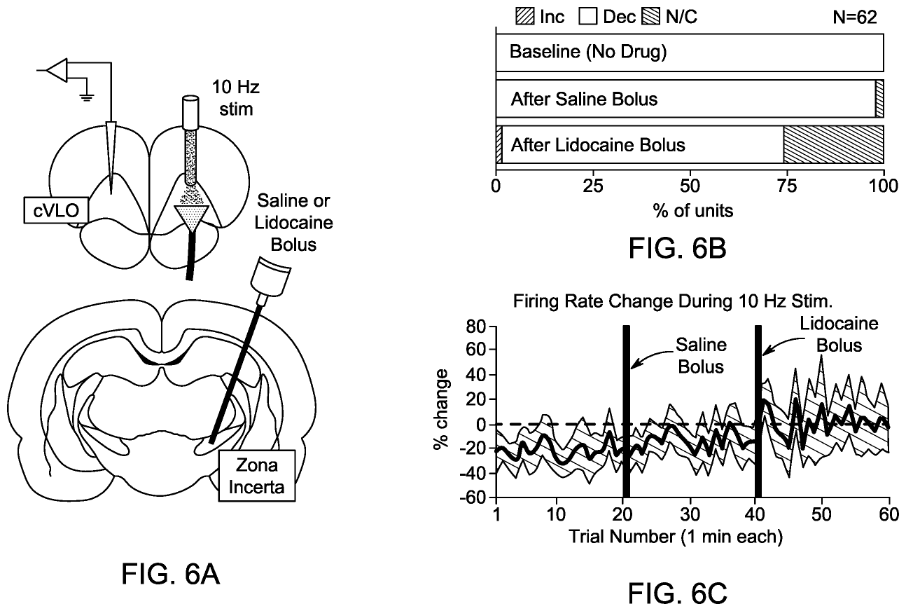

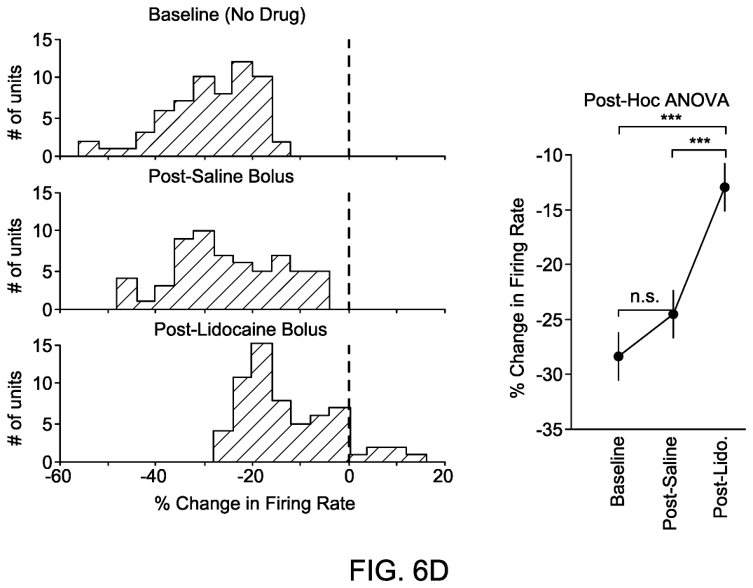

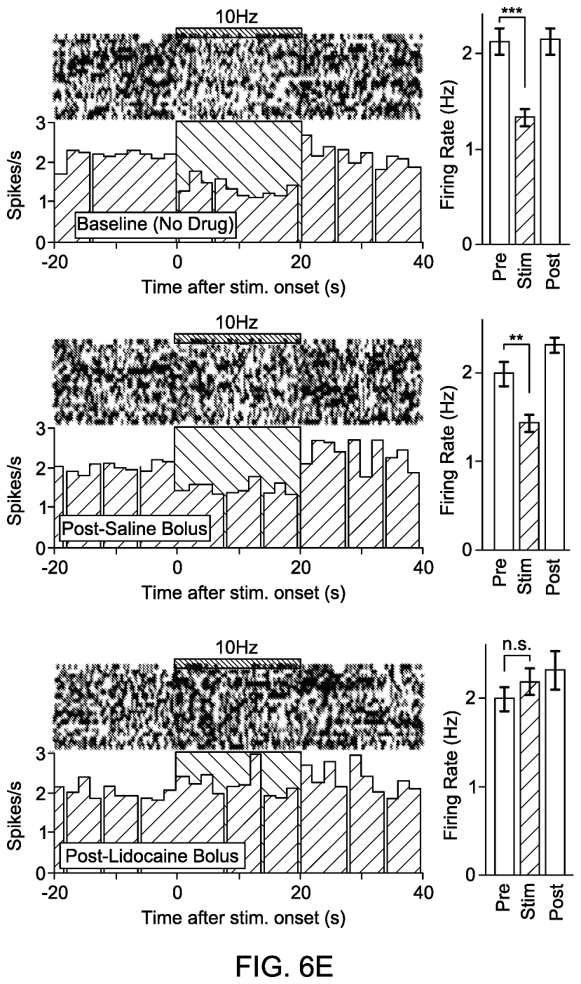

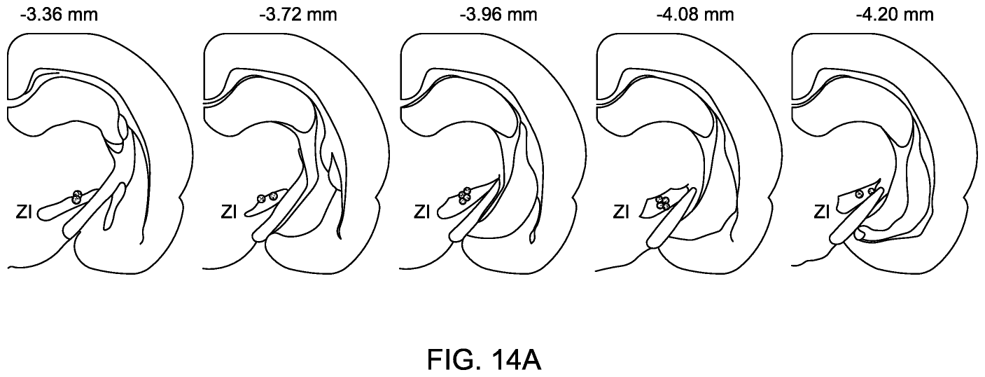

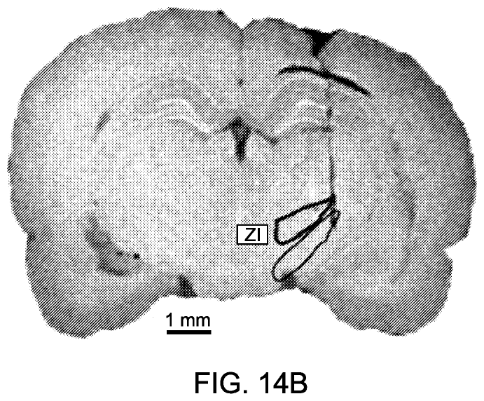

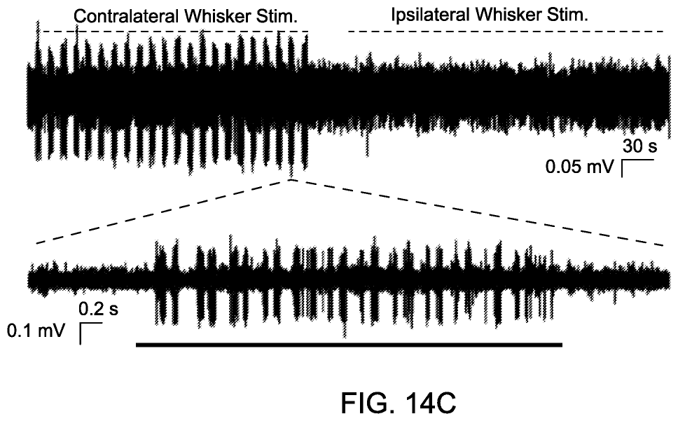

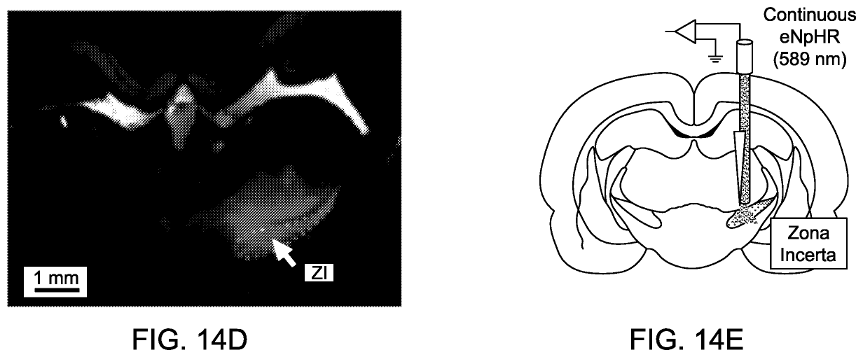

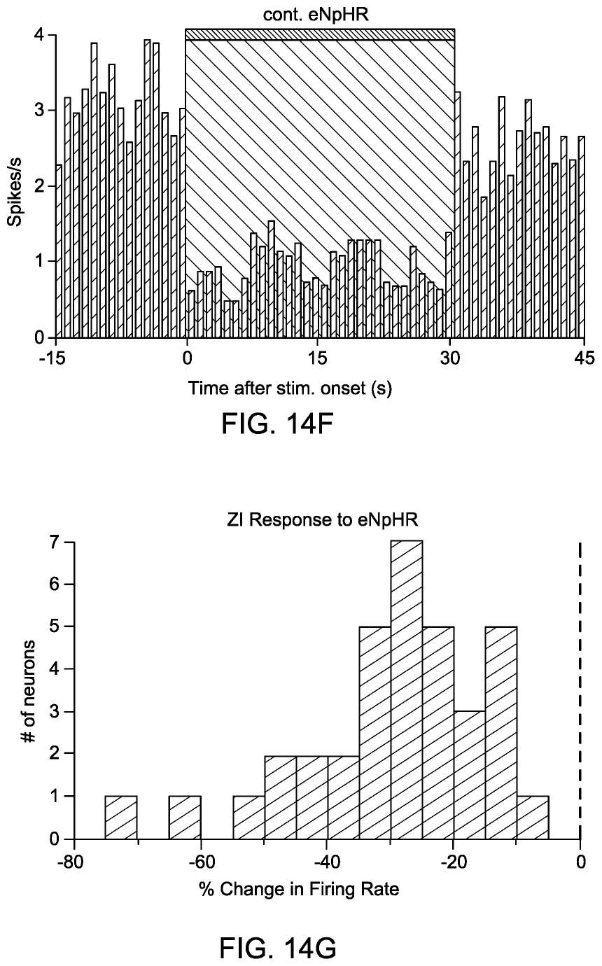

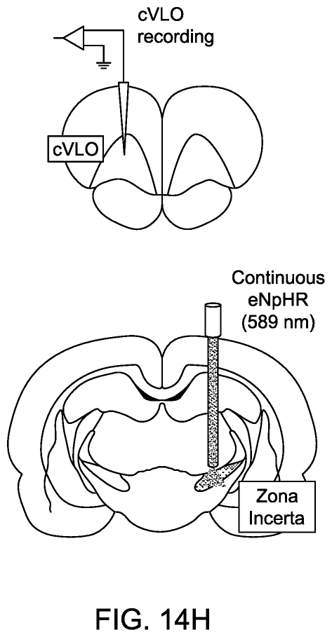

ZI activity was first inactivated via incertal infusions of the sodium channel blocker lidocaine hydrochloride (FIG. 6A and FIGS. 14A-14B). Incertal saline infusion did not affect the remote inhibition driven by 10 Hz stimulation. At least 98% of units recorded in cVLO exhibited a significant decrease in firing rate during stimulation both prior to and after saline infusion (FIG. 6B; N=62 units, 20 trials per condition). In contrast, only 72% of units were inhibited during 10 Hz stimulation after inactivation of zona incerta with lidocaine (FIG. 6B). The median change in firing rate evoked by stimulation was also significantly different between the three conditions (FIG. 6D, p=1.1×10⁻¹², χ²=55.0, 185 degrees of freedom). Post-hoc tests confirmed that stimulus-evoked changes after saline infusion were not different than baseline values (p=0.17), while stimulus-evoked changes after lidocaine infusion were different than both baseline and post-saline values (p=9.6×10⁻¹⁰ and 3.0×10⁻⁷, respectively). The reduction in evoked inhibition took immediate effect after lidocaine infusion and persisted throughout the following twenty trials (FIG. 6C). Peri-event time histograms from a representative unit show that in a subset of recorded units, incertal infusions of lidocaine, but not saline, completely eliminated the remote cortical inhibition driven by 10 Hz stimulation (FIG. 6E).

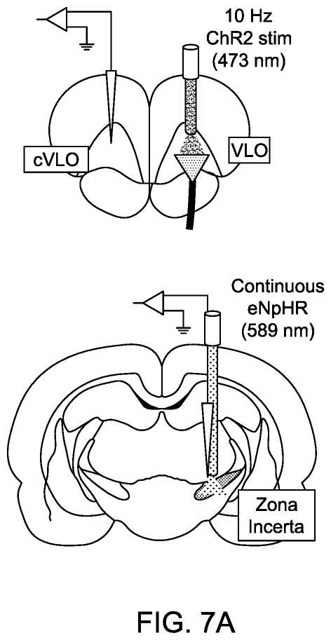

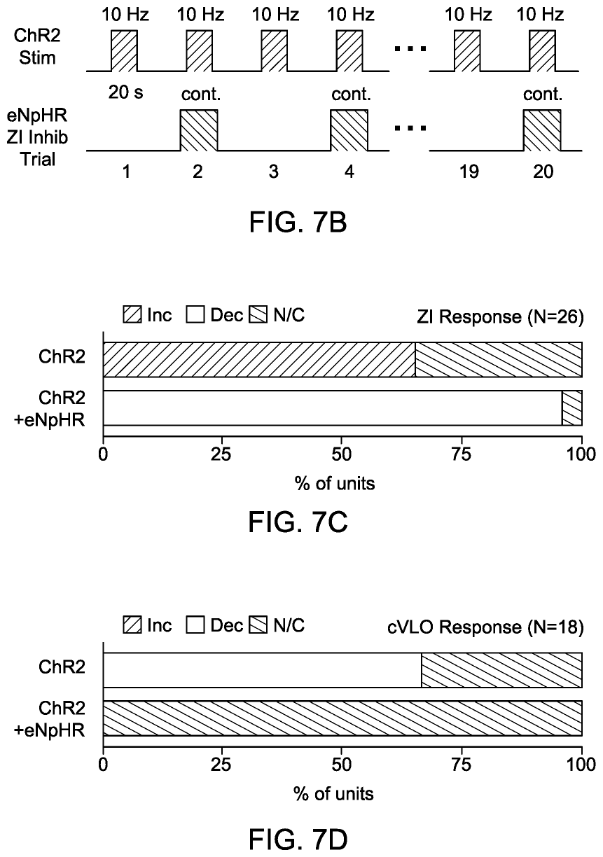

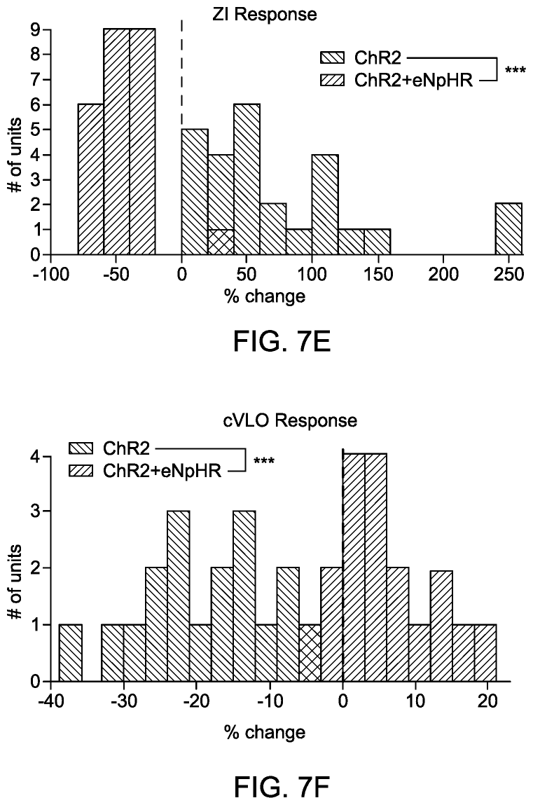

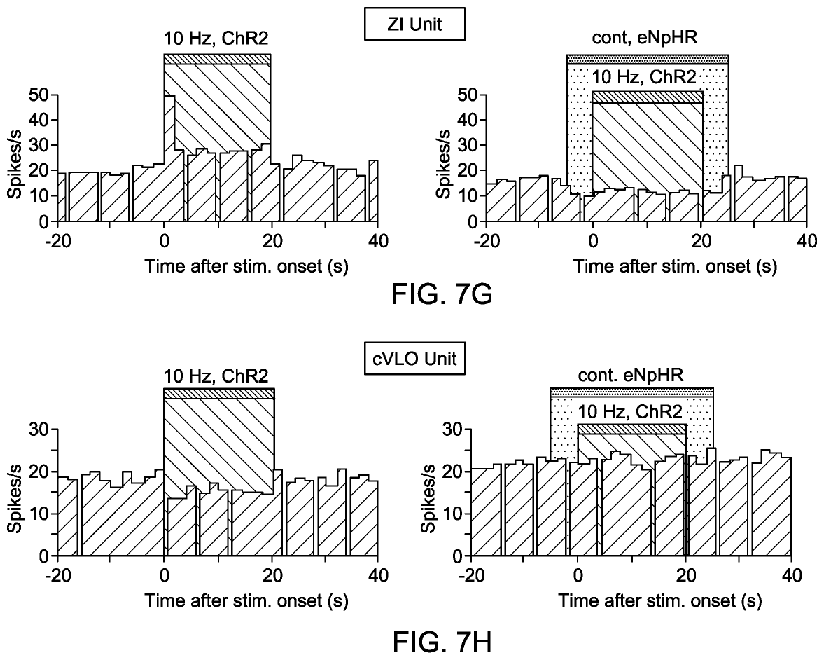

To confirm the role of ZI in mediating remote cortical inhibition, a similar experiment was performed using the inhibitory opsin eNpHR. In addition to the normal ChR2-EYFP injection into thalamus, adeno-associated virus carrying eNpHR-mCherry controlled by the pan-neuronal hSyn promoter was injected into zona incerta (FIG. FIG. 14D). This enabled robust suppression of incertal activity (FIGS. 14E-14G). To assess the role of ZI in remote inhibition, single-unit recordings were performed within cVLO and zona incerta during 10 Hz stimulation with and without concurrent eNpHR activation (FIG. 7A). These stimulation patterns were interleaved to ensure that differences between them could be attributed to eNpHR activation (FIG. 7B). Optrode placement in zona incerta was validated by confirming that the recorded population responded to contralateral whisker stimulation, a property that ZI is known to possess (Nicolelis et al., 1992, Brain Res 577, 134-141) (FIG. 14C).

Among 26 units recorded in zona incerta, 65% exhibited a significant increase in firing rate during 10 Hz thalamocortical stimulation (FIGS. 7C,7G). Thus, zona incerta was recruited during the stimulation paradigm that drove widespread inhibition. Concurrent activation of eNpHR during thalamocortical stimulation disrupted this recruitment. In 96% of recorded units, the firing rate during 10 Hz stimulation was actually less than pre-stimulus levels (FIG. 7C,G). The median change in incertal firing rate during 10 Hz stimulation was also significantly different between eNpHR and non-eNpHR trials (FIG. 7E).

To determine whether disruption of ZI recruitment affected the remote inhibition driven by thalamocortical stimulation, the change in cVLO's firing rate was quantified during eNpHR and non-eNpHR trials. 67% of units exhibited a significant decrease in firing rate during 10 Hz thalamocortical stimulation (FIGS. 7D,7H, N=18 units, 10 trials). Strikingly, suppression of ZI activity with eNpHR reversed this effect. No units exhibited a significant change in firing rate when 10 Hz stimulation was paired with eNpHR activation (FIGS. 7D,7H). The median change in firing rate across recorded units was also significantly different between the two conditions (FIG. 7F). Collectively, these data confirm that zona incerta mediates inhibition in at least one downstream region driven by low-frequency input to VLO.

To confirm the role of ZI in mediating inhibition, the effect of suppressing its activity alone on cortical firing (i.e. without thalamocortical stimulation) was investigated. Recordings were performed in cVLO (FIG. 14H), where a majority of units (N=22/24, 92%) exhibited no significant change during inhibition of zona incerta (FIG. 14I). The remaining 8% exhibited a small but significant increase in firing rate. These data suggest that any tonic inhibition provided by ZI over cortex is not enough to account for the large decreases in firing rate that occur during thalamocortical stimulation. As such, the role of zona incerta in mediating the observed inhibition must be specifically linked to VLO afferent stimulation.

### Discussion

### Role of ZI in Mediating Cortical Inhibition

It was discovered that thalamic input to VLO drives robust inhibitory effects in downstream regions, including the contralateral hemisphere.

By pairing inactivation of zona incerta with electrophysiology, it was found that stimulus-evoked inhibition in at least one cortical region is dependent on normal incertal processing. Future studies may combine incertal suppression with fMRI to assess whether ZI mediates inhibition throughout cortex. Previously, it was found that ZI inactivation reduced the degree of inhibition evoked in sensory cortex during 10 Hz stimulation of central thalamus (Liu et al., 2015, eLife 4, e09215). An important difference between these two studies is the spatial extent of inhibition. Unlike the widespread inhibition reported here, the negative fMRI signals driven by central thalamus stimulation were strictly localized to sensory cortex. This difference may be due to ZI's topographic organization. Anterograde studies have shown that the density of incertocortical terminals is strongest in sensory cortex (Lin et al., 1997, Neuroscience 81, 641-651). Furthermore, in a previous study, the thalamic projections that putatively drove incertal activity terminated in the dorsolateral ZI (Liu et al., 2015, eLife 4, e09215)—the same subregion of ZI that exhibits GABAergic projections to sensory cortex (Lin et al., 1990, Science 248, 1553-1556). The widespread nature of inhibition observed here suggests a broader activation of zona incerta covering multiple topographically organized subregions, perhaps supported by the extensive interconnections within ZI (Power and Mitrofanis, 1999, Neurosci Lett 267, 9-12).

The results of the present study show that incertal neurons exhibit frequency-dependent resonant properties or that different oscillatory patterns activate distinct cortico-incertal projections. A similar mechanism may explain the finding that widespread inhibition is evoked during low-, but not high-, frequency thalamocortical stimulation.

It is worth considering alternative mechanisms for the widespread inhibition evoked throughout cortex. Thalamic input to VLO might recruit nearby GABAergic projection neurons within cortex (Tamamaki and Tomioka, 2010). However, this population preferentially receives input from within cortex, and there is currently no evidence suggesting that thalamic projections would recruit such neurons (Tomioka et al., 2005). Downstream inhibition might also occur via cortico-cortical feedforward inhibition. It is well documented that spiking in cortical excitatory neurons can suppress nearby principal neurons via GABAergic interneurons in a frequency-dependent manner (Berger et al., 2009, Journal of physiology 587, 5411-5425; Silberberg and Markram, 2007, Neuron 53, 735-746). Unlike the findings of the present study though, these studies show that high frequencies of stimulation generate the strongest inhibitory response. The use of slice preparations to characterize this phenomenon also makes it difficult to generalize such behavior to long-range connections.

### Functional Role of Orbital Networks

The VLO takes part in a negative feedback loop responsible for descending pain modulation via the midbrain and spinal cord. It represents affective or arousing aspects of pain, and imaging studies indicate that it supports arousal. The results of the present study build upon these studies by showing that thalamic input to VLO is capable of dynamically controlling forebrain activation and deactivation, which reflect states of heightened and reduced arousal, respectively. Pain signals transmitted through VLO may follow two pathways—one descending through the canonical midbrain-spinal cord pathway and one within forebrain via striatum, thalamus, cortex, and zona incerta. The frequency-dependent polarity of cortical responses suggests that thalamic input to VLO can both facilitate and suppress these behavioral responses. The data of the present study show that OFC networks support the transformation of ascending thalamocortical signals to downstream inhibition.

Beyond inhibition, the results of the present study link anatomical and physiological studies on orbitofrontal networks to quantitative measurements of downstream activation. Both low and high frequencies of thalamocortical stimulation within VLO drove robust activation of the ipsilateral striatum. This activation could be mediated by striatal collaterals of the stimulated projection, or poly-synaptically via projections from orbitofrontal cortex to striatum. Until recently, this latter pathway had been relatively overlooked. However, accumulating evidence indicates a mediolateral topographic projection from the various sectors of orbital cortex to the caudate-putamen. Tracing studies show that projections from VLO terminate centrally in the caudate-putamen. This pathway may allow sensory information ascending through VLO to interact with prelimbic and cingulate networks that converge on the same striatal region (Groenewegen and Uylings, 2010). The fMRI data of the present study supports this approximate mapping, with low-frequency thalamocortical stimulation in VLO driving activation of the medial to central striatum. At higher frequencies of stimulation, activations covered almost all of striatum, suggesting the recruitment of local striatal circuits or other cortico-striatal pathways.

### Implications for Deep Brain Stimulation (DBS)

Deep brain stimulation within OFC has been explored as potential treatment for neurological disorders, but with mixed success. First, stimulations of VLO was shown to drive completely opposite effects throughout cortex, depending on the precise frequency used. Frequency is often a key parameter in optimizing the efficacy of DBS, and the present study illustrates this with dramatic effect. Second, stimulating different neuronal elements within the VLO circuit was shown to elicit distinct responses across the brain. One limitation of DBS is that it cannot separately modulate different neuronal elements within a region. This makes it difficult to identify exact mechanisms of different DBS paradigms. By using optogenetics to selectively stimulate thalamocortical projections, thalamic relay neurons, and cortical projection neurons, it was found that each element drives unique brain-wide responses.

### Significance for Neurovascular Coupling

Extracellular recordings were performed in this study to confirm that fMRI signals reflect underlying neuronal activity. They also offer important insight into the nature of neurovascular coupling.

The present study found that the frequency-dependent responses measured with CBV-weighted fMRI—both positive and negative—reflect corresponding changes in neuronal firing rate (CBV polarity was referred to as the polarity after inverting the raw time series, so that a positive signal reflects increases in CBV, and vice versa). Especially noteworthy is that negative CBV signals were linked to neuronal inhibition. The neuronal interpretation of negative fMRI signals can be complex, given the variety of possible causes for regional inhibition and their different metabolic demands. Negative CBV and BOLD signals have both been linked to increases in neuronal spiking and LFP (Englot et al., 2008, J Neurosci 28, 9066-9081; Mishra et al., 2011, J Neurosci 31, 15053-15064; Schridde et al., 2008, Cerebral cortex 18, 1814-1827; Shih et al., 2009, J Neurosci 29, 3036-3044), and more recent studies have reported instances where decreases in cortical CBV are not associated with any changes in activity (Hu and Huang, 2015, J Neurophysiol 114, 2152-2161; Ma et al., 2017, Neurosci Lett 637, 161-167). The results of the present study support such findings and extend them to CBV, which is becoming increasingly common in preclinical fMRI studies.

### Materials and Methods

### Animals

Healthy female Sprague-Dawley rats (12-14 weeks old at injection; Charles River, Wilmington, Mass., RRID:RGD_734476) were used for all experiments. The average age of animals was 42 and 45 weeks for fMRI scanning and electrophysiology, respectively. Animals were not used previously in any other procedures. Animals were group-housed prior to surgery and individually housed after surgery under a 12 hour light-dark cycle. Animals were provided with food and water ad libitum. Animal husbandry and experimental manipulation were performed in strict accordance with National Institute of Health and Stanford University Institutional Animal Care and Use Committee (IACUC) guidelines.

### Viral Injections and Fiber Placement

Injections were performed with concentrated rAAV5-CaMKII-hChR2(H134R)-EYFP virus produced at the University of North Carolina vector core (8.5×10¹² titer, lot #AV4316LM). During the injection procedure, rats were anesthetized with 2% isoflurane (Sigma-Aldrich, MO, USA) and secured in a stereotactic frame. Body temperature was maintained at 37° C. using a thermoresistive heating pad (FHC, Inc., ME, USA). Standard procedures for sterile surgery were followed. To prevent desiccation, artificial tears were applied to the eyes. After shaving the head, a triple scrub of 70% ethanol alternated with Betadine was applied. 200 μL of 0.5% bupivacaine was injected under the scalp. Slow-release buprenorphine was administered subcutaneously to minimize post-operative discomfort. Following a midline scalp incision, small craniotomies were performed with a dental drill above the submedial nucleus (−2.4 mm AP, +0.7 mm ML, −6.5 mm DV) and/or ventrolateral orbital cortex (+4.7 mm AP, +1.8 mm ML, −4.3 mm DV). Two microliters of virus were injected to the target region through a 10 mm 33 gauge beveled NanoFil needle (World Precision Instruments Inc., FL, USA) with a Micro4 microsyringe pump controller. For halorhodopsin experiments, 500 nL of rAAV5-hSyn-eNpHR3.0-mCherry-WPRE virus were injected into the right zona incerta after completion of the ChR2 injection (−3.96 mm AP, +2.8 mm ML, +7.4 mm DV; 6.7×10¹² titer, lot #AV4834B from University of North Carolina at Chapel Hill vector core). Following completion of injection(s), custom-made 200 m diameter fiber-optic implants (Thorlabs, Inc., NJ, USA, #FT200EMT; (Duffy et al., 2015, NeuroImage 123, 173-184)) were inserted and mounted to the skull using Clearfil AP-X light-cured dental cement (Kuraray Noritake Dental Inc., Japan, #1721-KA). This step was skipped in animals used for electrophysiology at the site of stimulation to allow for recording with an acute optrode. After suturing the incision, antiseptic Dermachlor rinse (Henry Schein, NY, USA) and 2% lidocaine hydrochloride jelly (Akorn Pharmaceuticals, IL, USA) were topically applied. Animals were kept on a heating pad until recovery from anesthesia.

### Optogenetic Functional MRI Data Acquisition

fMRI was performed in a 7T Bruker BioSpec small animal MRI system at Stanford University equipped with an 86 mm inner diameter transmit volume coil and a 2 cm inner diameter single-loop receive surface coil. Animals were initially anesthetized with 3-4% isoflurane and injected with 15 mg/kg of the contrast agent Feraheme (AMAG Pharmaceuticals, Inc., MA, USA) via tail vein before being secured in the MRI cradle. A 200 m diameter optical fiber was connected to a 473 nm laser source (LaserGlow Technologies, Toronto, Canada) and coupled with the fiber-optic implant. A single ofMRI scan consisted of a block design with 30 s of baseline measurements followed by six 20 s pulse trains of light delivered once per minute over 6 minutes. For the primary comparison of 10 versus 40 Hz stimulation (FIGS. 1A-1G and FIGS. 10A-10E), 4-7 scans were typically collected per frequency for each animal in a single session. For secondary frequency sweep experiments (FIGS. 2A-2H), 1-3 scans were collected for each frequency. For control experiments in FIGS. 12A-12D, 7-9 scans were collected for each frequency. With the exception of control experiments presented in FIGS. 12A-12D, a duty cycle of 30% was used across frequencies to maintain the total amount of light delivery. Stimulation frequencies were randomized across imaging sessions. Optical power was calibrated to 5 mW at the implanted fiber's tip. In two of the animals used for FIGS. 12A-12D, a higher power level was used (no more than −2×) to account for fibers implanted in a relatively dorsal position in VLO.

During fMRI scanning, animals were anesthetized with a mixture of 02 (35%), N20 (65%), and isoflurane (−1.5%). To ensure stable fMRI signals, body temperature was maintained at 37° C. using heated airflow. T2-weighted high-resolution anatomical images were acquired with a fast spin echo (RARE) sequence prior to fMRI scanning to check for brain damage and validate the optical fiber's location [0.14×0.14×0.5 mm³ spatial resolution, 256×256 matrix size, 2500 ms TR, 33 ms TE, 30 slices, 90° flip angle](FIG. 8D). A spiral sequence was used to acquire fMRI images during photostimulation with the following parameters: 35×35 mm² in-plane field of view, 0.5×0.5×0.5 mm³ spatial resolution, 4 interleaves, 300 flip angle, 750 ms TR, 12 ms echo time, and 23 slices (FIG. 1B). For some experiments, additional slices were acquired to facilitate image registration. Images were zero-padded in k-space to a 128×128 matrix size. Motion correction was performed using a GPU-based inverse Gauss-Newton algorithm to optimize detection of evoked responses (Fang and Lee, 2013). Scans with significant motion, identified by careful visual inspection for spiral artifacts and activations at the boundary of the brain, were excluded from analysis. Fewer than 2% of collected scans were excluded for this reason. Given that animals are anesthetized during imaging, these artifacts are likely due to occasional large breaths that distort the magnetic field.

### In Vivo Electrophysiology

In vivo electrophysiology was performed to directly measure the neuronal activity of various brain regions during thalamocortical stimulation. As with imaging, anesthesia was maintained with a mixture of 02 (35%), N20 (65%), and −1.5% isoflurane. Throughout the procedure, body temperature was maintained at 37° C. using a thermoresistive heating pad (FHC, Inc., ME, USA). After securing the animal within a stereotactic frame, a 16-channel microelectrode array (NeuroNexus Technologies, MI, USA; A1×16 standard model linear electrode array) was inserted at the desired recording site. For recordings at the site of stimulation and zona incerta, an optical fiber glued to the electrode tip was used to deliver light. Remote recordings were performed at the following coordinates, averaged across animals: contralateral VLO (+4.68 mm AP, −1.90 mm ML, −4.90 mm DV), ipsilateral motor cortex (+3.24 mm AP, +2.43 mm ML, −3.00 mm DV), ipsilateral reticular nucleus (−1.86 mm AP, +2.20 mm ML, −6.93 mm DV), contralateral reticular nucleus (−1.92 mm AP, −2.10 mm ML, −7.17 mm DV), and ipsilateral zona incerta (−4.00 mm AP, +3.10 mm ML, −7.13 mm DV). Light was delivered to the fiber-optic implant at VLO via a 473 nm laser source calibrated to 5 mW power delivery. In one animal, a higher power level was used (no more than −2×) to account for a fiber implanted in a relatively dorsal position in VLO. For the optrode positioned in zona incerta, a 200 m diameter optical fiber was used to deliver continuous light from a 589 nm laser source (LaserGlow Technologies) calibrated to 5 mW at the implanted fiber's tip. Recordings were performed for 20 s without stimulation, followed by repeated stimulation cycles (20 s on, 40 s off) at 10 or 40 Hz with a 30% duty cycle. To assess the role of zona incerta, 10 Hz stimulation trials were interleaved with 30 s periods of simultaneous eNpHR activation beginning 5 s prior to 10 Hz stimulation (FIG. 7B).

### Intracerebral Infusions

Pharmacological infusions were performed during in vivo electrophysiology (same procedure as described above) with bicuculline methiodide (BMI; 0.6 mg/ml; Sigma-Aldrich #14343), lidocaine hydrochloride (2%; Fresenius Kabi, IL, USA; #491507), or sterile saline as a control. Solutions were delivered at a rate of 250 nL/min via two polyethylene cannulas (0.011/0.024″ ID/OD; A-M Systems, WA, USA)—one for saline and one for the active pharmacological agent—glued to the tip of a recording electrode, directly above the topmost recording contact. Cannula tips were beveled to ensure that injected solutions were released in the direction of the electrode.

Infusions of saline and BMI (500 nL) were performed in the contralateral VLO while recording directly below the site of infusion (FIG. 5B). For each solution, twenty 10 Hz stimulation/recording trials were performed before infusion onset, and twenty more trials were performed immediately after infusion. Saline was delivered first, followed by BMI. To assess the role of zona incerta, saline and lidocaine (500-1000 nL) were delivered to the ipsilateral zona incerta while recording in the contralateral VLO. Twenty 10 Hz stimulation/recording trials were performed before any infusion onset, followed by saline infusion, twenty stimulation/recording trials, lidocaine infusion, and another twenty stimulation/recording trials.

### Immunohistochemistry

Standard immunohistochemistry techniques were used to amplify the endogenous EYFP signal fused to ChR2. Rats were deeply anesthetized with isoflurane and transcardially perfused with 0.1M phosphate-buffered saline (PBS) and ice-cold 4% paraformaldehyde (PFA) in PBS. Brains were extracted and fixed in 4% PFA overnight at 4° C. Brains were then equilibrated in 30% sucrose in PBS at 4° C. Coronal sections (40 m) were prepared on a freezing microtome. Free-floating sections were: [1] washed 5 times with PBS (10 mins each), [2] blocked and permeabilized with 5% normal donkey serum (NDS) and 0.4% Triton X-100 in PBS for 1 hr, [3] incubated at 4° C. overnight with primary antibody against chicken green fluorescent protein (1:1000; Aves, OR, USA; #GFP-1020, RRID:AB_2307313), [4] washed 7 times with a 2% NDS in PBS wash buffer (10 mins each), [5] incubated for 1 hr at room temperature with the secondary antibody Alexa Fluor 568 goat anti-chicken IgY (1:500; Thermo Fisher Scientific, MA, USA; #A-11041, R_RZID:AB_2534098), [6] washed 7 times with the wash buffer (10 mins each), [7] washed 2 times with PBS (20 mins each), [8] incubated with DAPI (0.002% DAPI [5 mg/ml] in PBS; Thermo Fisher Scientific, #D1306, RRID:AB 2629482) for 5 minutes, [9] washed 3 times with PBS (10 mins each), and [10] mounted with Fluoromount-G (SouthernBiotech, AL, USA; #0100-01). Immuno-fluorescence was assessed with a Zeiss laser confocal microscope. Antibodies were diluted with a solution of 5% NDS and 0.1% Tween-20 in PBS.

### Quantification and Statistical Analysis

Functional MRI Data Analysis:

fMRI data processing was performed with SPM12 (Ashburner et al., 2014, SPM12 manual. Wellcome Trust Centre for Neuroimaging, London, UK) in Matlab (MathWorks, Inc., MA, USA). Motion-corrected images belonging to the same stimulation frequency and scanning session were first spatially smoothed (0.4 mm FWHM Gaussian kernel) and averaged together. The average 4D images were then aligned to a common coordinate frame using affine and non-rigid transformations with NiftyReg (Modat et al., 2014, Journal of medical imaging 1, 024003; Modat et al., 2010, Computer methods and programs in biomedicine 98, 278-284). Within each animal, an equal number of scans for each frequency were averaged together. For the frequency sweep experiments, one animal lacked 15 Hz data and another lacked 25 and 30 Hz data.

Fixed effect analyses were performed at the subject level using a general linear model. The design matrix (FIG. 1C) was created by convolving the stimulation paradigm with fourth-order gamma basis functions, which have been shown to be optimal for balancing detection and characterization of heterogeneous fMRI signals. For quantification of active brain volume at the single-subject level, active voxels were identified as those with a t-score magnitude greater than 3.16 (p<0.001, uncorrected). Fixed effect analyses were also performed at the group level to generate the activation maps in FIGS. 1-3. Voxels with a t-statistic magnitude corresponding to significant p-values were overlaid onto a T2-weighted anatomical image averaged across subjects. Warm colors in activation maps indicate positive t-scores, while cool colors indicate negative t-scores. Regions of interest visualized in FIG. 10A were defined by matching a superimposed digital rat brain atlas (Paxinos and Watson, 2006) to visible anatomical features.

Time series were calculated on a voxel-wise basis from each animal's average 4D image as the percent modulation of the fMRI signal relative to the 30 s baseline period collected prior to stimulation. Detrending was performed with a 1 minute moving average kernel. With the exception of FIGS. 9A-9D, which averaged over significantly modulated voxels only, time series were generated by averaging across animals the mean time series of all voxels in the corresponding region of interest. To better compare responses across frequencies in FIGS. 2A-2H, time series were vertically shifted to start at 0% change. The percent signal change calculated from the raw fMRI signal was also inverted in order to make increases in signal portray increases in CBV. YfMRI values in FIGS. 10A-10E and FIGS. 12A-12D were calculated at the sum of the fMRI response over all measured time points, excluding the 30 s baseline period collected before the first stimulation cycle (120 points over six minutes, in total).

Electrophysiology Analysis

Recordings were performed with either the OpenEphys recording system and GUI (Siegle et al., 2017, J Neural Eng 14) or Plexon OmniPlex system with PlexControl software (Plexon Inc., TX, USA). For the OpenEphys recordings, signals were collected at 30 kHz and band-pass filtered between 300 Hz and 6 kHz. Spike detection and clustering were performed with the Wave_Clus software package in Matlab, using wavelets and super-paramagnetic clustering (Quiroga et al., 2004, Neural Comput 16, 1661-1687). For recordings made with the Plexon system, the Plexon multichannel acquisition processor was used to amplify and band-pass filter the acquired signal between 150 Hz and 8 kHz. Signals were digitized at 40 kHz and processed to extract action potentials in real-time.

Statistics

Statistical tests were performed in Matlab. Exact values of N for all tests can be found in the main text, figures, and figure legends. For volumetric comparisons in FIGS. 10A-10E, two-tailed paired t-tests were used to identify changes in the amount of fMRI activation between 10 and 40 Hz. For comparisons of YfMRI against zero in FIGS. 10A-10E, two-tailed t-tests were applied. For in vivo electrophysiology, two-tailed paired t-tests were used to identify significant changes in firing rate within each unit between pre-stimulation and stimulation periods. For experiments with ChR2 stimulation only, these periods were 20 s each. For experiments with only eNpHR activation, the 30 s eNpHR period was compared with the 15 s pre-stimulus period. For experiments with both ChR2 stimulation and eNpHR activation, the 20 s ChR2 period was compared with the 15 s period before either stimulus was delivered. Independence was assumed between repeated electrophysiology trials. Histograms of percent changes in firing rate (FIGS. 4A-4H, FIGS. 5A-5I, FIG. 7 and FIG. 13A-13I) were compared using a two-tailed Mann-Whitney U-test across units since assumptions on normality could not be met. Histograms in FIGS. 6A-6D were compared using a one-way non-parametric ANOVA test (i.e. Kruskal-Wallis) with Tukey-Kramer post-hoc comparisons. For all histogram comparisons, data for each unit was obtained by averaging over repeated trials, and single-units were assumed to represent independent samples. Baseline firing rates in FIGS. 5A-5I were compared using a two-tailed t-test for individual units and a two-tailed paired t-test across all units. For all tests, variance was generally similar across groups being compared and significance was determined at the α=0.05 cutoff level.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-39. (canceled)

40. A system for modulating temporal patterns of neuronal activity in the brain of an individual, the system comprising:
i) a light source configured to stimulate, with a light pulse, one or more of thalamocortical projections, thalamic relay neurons, cortical projection neurons, cell bodies in a thalamic submedial nucleus, and cell bodies in the VLO in the brain of the individual, wherein a light-responsive opsin polypeptide is expressed in cell bodies in one or more of a ventrolateral orbitofrontal cortex (VLO) and a thalamus of the brain; and
ii) a functional magnetic resonance imaging (fMRI) device configured to scan the whole-brain during stimulation to produce an fMRI signal;
wherein a positive measured fMRI signal is associated with an increase in neuronal activity following stimulation, and wherein a negative measured fMRI signal is associated with a decrease in neuronal activity following stimulation.

41. The system of claim 40, wherein the whole-brain comprises an ipsilateral and contralateral brain region.

42. The system of claim 41, wherein the ipsilateral region comprises a left hemisphere of the brain comprising the a medial prefontal cortex, a lateral prefontal cortex, a motor cortex, a cingulate cortex, a sensory cortex, an insular cortex, a striatum, and a thalamus.

43. The system of claim 41, wherein the contralateral region comprises a right hemisphere of the brain comprising a medial prefontal cortex, a lateral prefontal cortex, a motor cortex, a cingulate cortex, a sensory cortex, an insular cortex, a striatum, and a thalamus.

44. The system of claim 40, wherein the light pulse has a frequency ranging from 5 Hz to 40 Hz.

45. The system of claim 44, wherein the light pulse has a frequency of 10 Hz or more.

46. The system of claim 44, wherein the light pulse has a frequency of 40 Hz or more.

47. The system of claim 40, wherein stimulation of the thalamocortical projection with the light pulse having a frequency of 10 Hz or more results in the negative measured fMRI signal in the sensory, motor, and cingulate cortex of the ipsilateral region of the brain.

48. The system of claim 40, wherein the light source can be reversibly inserted in the VLO of the individual.

49. The system of claim 40, wherein stimulation of the thalamocortical projection with the light pulse having a frequency of 40 Hz or more results in the positive measured fMRI signal.

50. The system of claim 40, wherein stimulation of the thalamocortical projection with the light pulse having a frequency of 10 Hz or more results in the negative measured fMRI signals in the contralateral region of the brain.

51. The system of claim 40, wherein stimulation of the cell bodies in the VLO with the light pulse ranging from 5 Hz to 40 Hz results in the positive measured fMRI signal in the ipsilateral region of the brain.

52. The system of claim 40, wherein the light-activated is expressed in layer I and layer III neurons of the VLO of the brain.

53. The system of claim 40, wherein the implantable light source is implanted in a dorsal position in the VLO of the brain.

54. The system of claim 40, wherein stimulation with the light pulse having a frequency ranging from 5 Hz to 10 Hz inhibits the neuronal activity of the ipsilateral thalamus of the brain.

55. The system of claim 40, wherein the fMRI signal comprises a cerebral blood volume (CBV).

56. The system of claim 40, wherein the system further comprises a second light-activated polypeptide expressed in neurons in a zona incerta region of the brain.

57. The system of claim 40, wherein the system further comprises electrophysiological recording device configured to detect firing rates of neurons in one or more brain regions associated with the measured fMRI signal.

58. The system of claim 57, wherein the one or more brain regions comprises the ipsilateral VLO of the brain.

59. The system of claim 58, wherein a positive fMRI signal is associated with an increased firing rate of neurons in the ipsilateral VLO of the brain.


SEQUENCE LISTING (64 sequences)

SEQ ID NO: 1 - protein, 310 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 2 - protein, 340 aa, Artificial Sequence
  synthetic sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVPAAAKSRITSEGEYIPLDQIDINVFCYENEV

SEQ ID NO: 3 - protein, 310 aa, Artificial Sequence
  synthetic sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 4 - protein, 340 aa, Artificial Sequence
  synthetic sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVPAAAKSRITSEGEYIPLDQIDINVFCYENEV

SEQ ID NO: 5 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 6 - protein, 330 aa, Artificial Sequence
  synthetic sequence
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDAAAKSRITSEGEYIPLDQIDINVFCYENEV

SEQ ID NO: 7 - protein, 344 aa, Artificial Sequence
  synthetic sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 8 - protein, 374 aa, Artificial Sequence
  synthetic sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDAAAKSRITSEGEYIPLDQIDINVFCYENEV

SEQ ID NO: 9 - protein, 348 aa, Chlamydomonas reinhardtii
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 10 - protein, 378 aa, Artificial Sequence
  synthetic sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVAAAKSRITSEGEYIPLDQIDINVFCYENEV

SEQ ID NO: 11 - protein, 350 aa, Chlamydomonas reinhardtii
MVSRRPWLLALALAVALAAGSAGASTGSDATVPVATQDGPDYVFHRAHERMLFQTSYTLENNGSVICIPNNGQCFCLAWLKSNGTNAEKLAANILQWVTFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESS

SEQ ID NO: 12 - protein, 380 aa, Artificial Sequence
  synthetic sequence
MVSRRPWLLALALAVALAAGSAGASTGSDATVPVATQDGPDYVFHRAHERMLFQTSYTLENNGSVICIPNNGQCFCLAWLKSNGTNAEKLAANILQWVTFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESSAAAKSRITSEGEYIPLDQIDINVFCYENEV

SEQ ID NO: 13 - protein, 316 aa, Artificial Sequence
  synthetic sequence
MGGAPAPDAHSAPPGNDSAGGSEYHAPAGYQVNPPYHPVHGYEEQCSSIYIYYGALWEQETARGFQWFAVFLSALFLAFYGWHAYKASVGWEEVYVCSVELIKVILEIYFEFTSPAMLFLYGGNITPWLRYAEWLLTCPVILIHLSNITGLSEEYNKRTMALLVSDLGTICMGVTAALATGWVKWLFYCIGLVYGTQTFYNAGIIYVESYYIMPAGGCKKLVLAMTAVYYSSWLMFPGLFIFGPEGMHTLSVAGSTIGHTIADLLSKNIWGLLGHFLRIKIHEHIIMYGDIRRPVSSQFLGRKVDVLAFVTEEDKV

SEQ ID NO: 14 - protein, 346 aa, Artificial Sequence
  synthetic sequence
MGGAPAPDAHSAPPGNDSAGGSEYHAPAGYQVNPPYHPVHGYEEQCSSIYIYYGALWEQETARGFQWFAVFLSALFLAFYGWHAYKASVGWEEVYVCSVELIKVILEIYFEFTSPAMLFLYGGNITPWLRYAEWLLTCPVILIHLSNITGLSEEYNKRTMALLVSDLGTICMGVTAALATGWVKWLFYCIGLVYGTQTFYNAGIIYVESYYIMPAGGCKKLVLAMTAVYYSSWLMFPGLFIFGPEGMHTLSVAGSTIGHTIADLLSKNIWGLLGHFLRIKIHEHIIMYGDIRRPVSSQFLGRKVDVLAFVTEEDKVAAAKSRITSEGEYIPLDQIDINVFCYENEV

SEQ ID NO: 15 - protein, 350 aa, Artificial Sequence
  synthetic sequence
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 16 - protein, 380 aa, Artificial Sequence
  synthetic sequence
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAAKSRITSEGEYIPLDQIDINVFCYENEV

SEQ ID NO: 17 - protein, 345 aa, Artificial Sequence
  synthetic sequence
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 18 - protein, 375 aa, Artificial Sequence
  synthetic sequence
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAAKSRITSEGEYIPLDQIDINVFCYENEV

SEQ ID NO: 19 - protein, 325 aa, Artificial Sequence
  synthetic sequence
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGV

SEQ ID NO: 20 - protein, 355 aa, Artificial Sequence
  synthetic sequence
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGVAAAKSRITSEGEYIPLDQIDINVFCYENEV

SEQ ID NO: 21 - protein, 258 aa, Halorubrum sodomense
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAAD

SEQ ID NO: 22 - protein, 293 aa, Artificial Sequence
  synthetic sequence
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAADRPVVAAAAKSRITSEGEYIPLDQIDINVFCYENEV

SEQ ID NO: 23 - protein, 248 aa, Halorubrum sp. TP009
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEP

SEQ ID NO: 24 - protein, 248 aa, Artificial Sequence; identical to SEQ ID NO: 23
  synthetic sequence

SEQ ID NO: 25 - protein, 242 aa, Guillardia theta
MLVGEGAKLDVHGCKTVDMASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESI

SEQ ID NO: 26 - protein, 272 aa, Artificial Sequence
  synthetic sequence
MLVGEGAKLDVHGCKTVDMASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESIAAAKSRITSEGEYIPLDQIDINVFCYENEV

SEQ ID NO: 27 - protein, 262 aa, Oxyrrhis marina
MAPLAQDWTYAEWSAVYNALSFGIAGMGSATIFFWLQLPNVTKNYRTALTITGIVTLIATYHYFRIFNSWVAAFNVGLGVNGAYEVTVSGTPFNDAYRYVDWLLTVPLLLVELILVMKLPAKETVCLAWTLGIASAVMVALGYPGEIQDDLSVRWFWWACAMVPFVYVVGTLVVGLGAATAKQPEGVVDLVSAARYLTVVSWLTYPFVYIVKNIGLAGSTATMYEQIGYSAADVTAKAVFGVLIWAIANAKSRLEEEGKLRA

SEQ ID NO: 28 - protein, 292 aa, Artificial Sequence
  synthetic sequence
MAPLAQDWTYAEWSAVYNALSFGIAGMGSATIFFWLQLPNVTKNYRTALTITGIVTLIATYHYFRIFNSWVAAFNVGLGVNGAYEVTVSGTPFNDAYRYVDWLLTVPLLLVELILVMKLPAKETVCLAWTLGIASAVMVALGYPGEIQDDLSVRWFWWACAMVPFVYVVGTLVVGLGAATAKQPEGVVDLVSAARYLTVVSWLTYPFVYIVKNIGLAGSTATMYEQIGYSAADVTAKAVFGVLIWAIANAKSRLEEEGKLRAAAAKSRITSEGEYIPLDQIDINVFCYENEV

SEQ ID NO: 29 - protein, 313 aa, Leptosphaeria maculans
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGA

SEQ ID NO: 30 - protein, 351 aa, Artificial Sequence
  synthetic sequence
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGARPVVAVSKAAAKSRITSEGEYIPLDQIDINVFCYENEV

SEQ ID NO: 31 - protein, 291 aa, Natronomonas pharaonis
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 32 - protein, 320 aa, Artificial Sequence
  synthetic sequence
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINFCYENEV

SEQ ID NO: 33 - protein, 303 aa, Artificial Sequence
  synthetic sequence
MVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINFCYENEV

SEQ ID NO: 34 - protein, 365 aa, Dunaliella salina
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDV

SEQ ID NO: 35 - protein, 395 aa, Artificial Sequence
  synthetic sequence
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDVAAAKSRITSEGEYIPLDQIDINVFCYENEV

SEQ ID NO: 36 - protein, 348 aa, Artificial Sequence
  synthetic sequence
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 37 - protein, 378 aa, Artificial Sequence
  synthetic sequence
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 38 - protein, 348 aa, Artificial Sequence
  synthetic sequence
  misc_feature (167)..(167): Xaa can be any naturally occurring amino acid
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTXPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 39 - protein, 378 aa, Artificial Sequence
  synthetic sequence
  misc_feature (167)..(167): Xaa can be any naturally occurring amino acid
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTXPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 40 - protein, 309 aa, Artificial Sequence
  synthetic sequence
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 41 - protein, 339 aa, Artificial Sequence
  synthetic sequence
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 42 - protein, 310 aa, Artificial Sequence
  synthetic sequence
MDYGGALSAVGRELLFVTNPVVVNGSVLVPEDQCYCAGWIESRGTNGAQTASNVLQWLSAGFSILLLMFYAYQTWKSTCGWEEIYVCAISMVKVILEFFFSFKNPSMLYLATGHRVKWLRYASWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVP

SEQ ID NO: 43 - protein, 340 aa, Artificial Sequence
  synthetic sequence
MDYGGALSAVGRELLFVTNPVVVNGSVLVPEDQCYCAGWIESRGTNGAQTASNVLQWLSAGFSILLLMFYAYQTWKSTCGWEEIYVCAISMVKVILEFFFSFKNPSMLYLATGHRVKWLRYASWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVPAAAKSRITSEGEYIPLDQIDINVFCYENEV

SEQ ID NO: 44 - protein, 344 aa, Artificial Sequence
  synthetic sequence
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 45 - protein, 374 aa, Artificial Sequence
  synthetic sequence
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEEDAAAKSRITSEGEYIPLDQIDINVFCYENEV

SEQ ID NO: 46 - protein, 305 aa, Artificial Sequence
  synthetic sequence
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 47 - protein, 335 aa, Artificial Sequence
  synthetic sequence
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEEDAAAKSRITSEGEYIPLDQIDINVFCYENEV

SEQ ID NO: 48 - protein, 350 aa, Artificial Sequence
  synthetic sequence
MVSRRPWLLALALAVALAAGSAGASTGSDATVPVATQDGPDYVFHRAHERMLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 49 - protein, 380 aa, Artificial Sequence
  synthetic sequence
MVSRRPWLLALALAVALAAGSAGASTGSDATVPVATQDGPDYVFHRAHERMLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESSAAAKSRITSEGEYIPLDQIDINVFCYENEV

SEQ ID NO: 50 - protein, 310 aa, Artificial Sequence
  synthetic sequence
MDYGGALSAVGLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 51 - protein, 340 aa, Artificial Sequence
  synthetic sequence
MDYGGALSAVGLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESSAAAKSRITSEGEYIPLDQIDINVFCYENEV

SEQ ID NO: 52 - protein, 5 aa, Artificial Sequence
  synthetic sequence
  VARIANT (2)..(3): Where each X can individually be any amino acid
VXXSL

SEQ ID NO: 53 - protein, 5 aa, Artificial Sequence
  synthetic sequence
VKESL

SEQ ID NO: 54 - protein, 5 aa, Artificial Sequence
  synthetic sequence
VLGSL

SEQ ID NO: 55 - protein, 16 aa, Artificial Sequence
  synthetic sequence
NANSFCYENEVALTSK

SEQ ID NO: 56 - protein, 20 aa, Artificial Sequence
  synthetic sequence
KSRITSEGEYIPLDQIDINV

SEQ ID NO: 57 - protein, 6 aa, Artificial Sequence
  synthetic sequence
  VARIANT (2)..(2): Wherein X can be any amino acid.
FXYENE

SEQ ID NO: 58 - protein, 7 aa, Artificial Sequence
  synthetic sequence
FCYENEV

SEQ ID NO: 59 - protein, 26 aa, Artificial Sequence
  synthetic sequence
MDYGGALSAVGRELLFVTNPVVVNGS

SEQ ID NO: 60 - protein, 27 aa, Artificial Sequence
  synthetic sequence
MAGHSNSMALFSFSLLWLCSGVLGTEF

SEQ ID NO: 61 - protein, 18 aa, Artificial Sequence
  synthetic sequence
MRGTPLLLVVSLFSLLQD

SEQ ID NO: 62 - protein, 18 aa, Artificial Sequence
  synthetic sequence
MTETLPPVTESAVALQAE

SEQ ID NO: 63 - protein, 11 aa, Artificial Sequence
  synthetic sequence
MDYGGALSAVG

SEQ ID NO: 64 - protein, 23 aa, Artificial Sequence
  synthetic sequence
MGLRALMLWLLAAAGLVRESLQG